SEQUENCE LISTING (106 sequences)

SEQ ID NO: 1 - DNA, 1089 bp, Micrococcus luteus
  source 1..1089: mol_type = other DNA
atgatcaagtacttgggtagcaagcggacgctcgtgcccgtcctcggtgacatcgcttcggcctctgaagcaacagaggcggttgacctgttcactggcacgacgcgtgtggcgcaagagttcaagcgtcgcgggcttcgagttcttgctaacgacatagcgacgtactccgaggttttagcccagtgctatatcgccaccaacggccaggaagttgaccgccgtgcgctcgaggccgctctggcggagctgaacgccttgcccggcgaacctggatacttcacggaaaccttctgtgaggcttctcgctacttccagcccaagaacggggctcgggtggatgcaatcaggaatgcgatcgacgaccggtacgcggactcatggatgcgaccgatcctcctcacgagcttgatgcttgcggccgaccgcgtcgactccactaccggagtgcagatggcttacctgaagcagtgggccgcgcgtgcgcacaatgatctagagttgcggcttccagacctaatcgcaggtgacggtgacgctgctcgtgaggatgcggtgactctcgcacaagagctgcctcgcgtccagctgatgtaccttgatcctccctataaccagcacaggtacttcaccaactaccatatttgggagaccctgattcgttgggatgcccctgagagttatgggatcgcctgtaagcgcattgactctcgagatgatgccaccaagagcccctataatatgaagcggcgaatgcccgacgagatgcgtcgcctgctgatgaccatcaaggcggacctcgcggttgtatcttacaacaatgagtcgtggattgatccggagacgatgatgtcgaccctgcgcgatgcgggatatgaggacgtgcgtctgctcgctttcgactataagcgctacgttggggctcaaatcgggatctacaatccctccggggaaaaggtcggtcgtgtgagtcacctccgaaacatcgagtatctctttcttgcgggaccaacggagcgcgttgaggtgtgcgccgcgagtgttgaacaccgagcactacccaaggaaccggaactcaccgcgttctag

SEQ ID NO: 2 - protein, 362 aa, Micrococcus luteus
MIKYLGSKRTLVPVLGDIASASEATEAVDLFTGTTRVAQEFKRRGLRVLANDIATYSEVLAQCYIATNGQEVDRRALEAALAELNALPGEPGYFTETFCEASRYFQPKNGARVDAIRNAIDDRYADSWMRPILLTSLMLAADRVDSTTGVQMAYLKQWAARAHNDLELRLPDLIAGDGDAAREDAVTLAQELPRVQLMYLDPPYNQHRYFTNYHIWETLIRWDAPESYGIACKRIDSRDDATKSPYNMKRRMPDEMRRLLMTIKADLAVVSYNNESWIDPETMMSTLRDAGYEDVRLLAFDYKRYVGAQIGIYNPSGEKVGRVSHLRNIEYLFLAGPTERVEVCAASVEHRALPKEPELTAF

SEQ ID NO: 3 - DNA, 1146 bp, Helicobacter pylori
  source 1..1146: mol_type = other DNA
ttggagaattttttgaataatttagatattaaaaccttagggcaggttttcacccctaaaaagatagtggatttcatgctcactctcaagcacaatcatgggagtgttttagagccaagcgcgggcgatgggagttttttaaagcgcttaaaaaaggctgtagggattgaaatcgatcctaaaatctgccctaaaaatgccctttgcatggacttttttgactaccctttagaaaatcaatttgacacgattattggcaatccgccctatgtcaagcacaaggatattgcgccaagcacgaaagaaaaactccattacagcctttttgatgaaaggagtaatctatacttgtttttcatagaaaaagcgatcaagcatttaaagcctaaaggcgaattgattttcatcaccccaagggattttttaaaatccacttctagcgtgaaattaaacgaatggatttacaaagaaggcacgataacgcatttttttgaattaggcgatcaaaagattttcccaaacgccatgcctaattgcgtgatttttcgtttttgtaaaggtgatttcagtagaatcaccaacgatggtttgcaatttgtgtgcaaaaaaggcattttgtatttcctcaaccaatcttacacgcaaaaattaagcgaggtttttaaggttaaggtgggggcagtgagcgggtgcgataagatttttaaaaatgaaacatacgggaatttagaatttgtcacctcaatcaccaaaagaaccaatgttttagaaaaaatggtttttgtcaataaacctaatgattatttactccagcataaagacagcttgatgcaaagaaagattaaaaaattcaatgaaagtaattggtttgaatgggggaggatgcatcacatatcccctaaaaaacgcatttatgttaacgccaaaacgcgccaaaaaaacccctttttcatccaccaatgccctaattatgacggctctattttagcgctattcccttataaccaaaatttggatttacaaaacctctgcgataaactcaacgctatcaactggcaagaattaggctttgtgtgcggcgggcgttttttgttttcgcagcgctctttagaaaacgcccttttgcctaaagactttttaaattag

SEQ ID NO: 4 - DNA, 609 bp, Mycoplasma fermentans
  source 1..609: mol_type = other DNA
atgggtaaatctgaattaagtggaagattaaattggcaagcattggctggattaaaagctagtggtgctgaacaaaacttatataacgtgtttaacgctgtttttgaaggaactaaatacgttttatacgagaagccaaagcaccttaaaaatctatacgctcaagtagtcttacctgatgatgttattaaagaaatttttaatcctttaattgatttatcaactactcaatggggtgtttctccagatttcgcaatagaaaatacagaaacgcataaaattctttttggtgaaattaaaagacaagatggatgggtagaaggtaaagatcctagtgctggcaggggtaatgcacatgagagatcttgtaaattatttactcctggattattaaaagcttatagaacaattggtggaattaacgatgaagagatattgccattctgggttgtattcgaaggtgatataacacgagatcccaaaagagtaagagaaattactttctggtatgaccactatcaagataattatttcatgtggcgaccaaatgaatcaggcgaaaaattagttcaacacttcaatgaaaaattaaaaaaatatttagattaa

SEQ ID NO: 5 - protein, 202 aa, Mycoplasma fermentans
MGKSELSGRLNWQALAGLKASGAEQNLYNVFNAVFEGTKYVLYEKPKHLKNLYAQVVLPDDVIKEIFNPLIDLSTTQWGVSPDFAIENTETHKILFGEIKRQDGWVEGKDPSAGRGNAHERSCKLFTPGLLKAYRTIGGINDEEILPFWVVFEGDITRDPKRVREITFWYDHYQDNYFMWRPNESGEKLVQHFNEKLKKYLD

SEQ ID NO: 6 - DNA, 1152 bp, Bacillus stearothermophilus
  source 1..1152: mol_type = other DNA
atggctattacattatgtgacataaatggttgtagacttgagagaggacatactggtaaacataataaatttcctgaatttgtatggacttctcaatttaataaaaaagatattgataaggtcaataaagcaggatatgcaacaccaagaggtggggacaaaggagcctatcagaaccatgtttacagaaataataaagtaattattccttttgaaaggttggaaaatgttaatttaaataactatcaagatggatatgttattaggttattccctaatcagtactttgaatcagccggggtagttaagccggaattcttacaaccaaattcatttgttaaagttggggacaatgcatttattttatatcgcacacattcatcttttgaggaattacctcctctaccagactgggaggttagacatctaaaaaagaacggtaatatagttaccagaagaagtaaggacgtaatcgatgctggacattatgtcttacgattatcatcaattagtaacaaaaaagaaagaaaagagggccctcctcaaggtatttttgcacctgaatatgcaaatgcagagactaattatctgtcaaaagcatttttagcctggttaattattaaaactcaaaatagtccgtataatgaagaacaattccaacacttaagagcgatcttaattagtcataatctcatcaatatttctcaacttgaagaaaaggctattctaaagaatggtatcacatgctgccctttatgcgagcaaattattttttacgaacagctacacgaaatggtttcttttgaaggtgcgtctggccttgcgaattcacaagaacaggttgagggtgcaactaggtcaacatcagttaatttattccatatggtaccattagtatatgaaaccttggaacacaaacctgatcaaatagcatggggccatgccatttgtaatactagacttggtcaaagagagtgcctgcctcttagtagactaaaacaagaaggtacgcccgttggtcttcttgatgaagattcgaatcttgaagtattaggatggattagtaaagataagcaatttattcgtacagaaaatggggaagtttggattaaaattacagatattgaatttaacgatgactttgaagaataa

SEQ ID NO: 7 - protein, 383 aa, Bacillus stearothermophilus
MAITLCDINGCRLERGHTGKHNKFPEFVWTSQFNKKDIDKVNKAGYATPRGGDKGAYQNHVYRNNKVIIPFERLENVNLNNYQDGYVIRLFPNQYFESAGVVKPEFLQPNSFVKVGDNAFILYRTHSSFEELPPLPDWEVRHLKKNGNIVTRRSKDVIDAGHYVLRLSSISNKKERKEGPPQGIFAPEYANAETNYLSKAFLAWLIIKTQNSPYNEEQFQHLRAILISHNLINISQLEEKAILKNGITCCPLCEQIIFYEQLHEMVSFEGASGLANSQEQVEGATRSTSVNLFHMVPLVYETLEHKPDQIAWGHAICNTRLGQRECLPLSRLKQEGTPVGLLDEDSNLEVLGWISKDKQFIRTENGEVWIKITDIEFNDDFEE

SEQ ID NO: 8 - DNA, 1536 bp, Bacillus stearothermophilus
  source 1..1536: mol_type = other DNA
atgatttttgctgatattgaatttgaaaaagaacttttttcagctgctaataaattaaggggaaaaattgctccaagtgagtataagcattatgttttgcctttgatattccttagatatttatctcttaaataccaacaaagaaggaatgaaattcaacaacagataaatgattcaagggatcacaagaaaaatcaagatgaagtgttaaagatattggaagacaggactgaatacaccaaagtaaatgttttctatattcctgaaaaagctagttgggaatacttattgaaaaattccgaaaatgataaaattaaagaaatgatagattcagctatggaaatactggaaaatgaatatgacgagttaaaaggtgttttgccaaagatatataaaaactcaaatataccgaatgaagttattagtgatttactaaaactattttctcaagaagtattttcagcacatgatggaagaaatgttgatttattggggagagtttatgaatactttataagtaattttgctactacagaaggtactagaggtggtgaatattttacaccgtcttcaatcgtaaaattattggtagcaatgctagagcccattaaaggtacagtttatgatccggcctgtgggacaggaggaatgtttattcagtctaataaatatagagaaaataatcataacttgtgttttgtaggccaggaacaaaacgagcttactatcaaattggctaaaatgaatggaattctacatggaataaatcctgaaattagacaaggtgattcattattaaatgaccgttatccagaattgaaagctgaaattgtaatatctaatccaccgtttaatatgaaggattggggagctgaacgcctgccacttaatgataagcgattaataggaccggtaacaaacagtaatgcaaattacatgtggatacagcattttctataccatttaaaagatggtggtttagcaggatttgttattgctaatggagctttgactagtaatctggctgctgaaaaaattgtaaggaaacacttaatagacaatgattatgtagattgtgttgttcaattacctgaaaaaatgttctttggtactggcattccaagtgctttagtgtttttaagtaagaatcgaaatggaagtaacggccatgccaaaagagaaaaagaggttctatttattgatgcaagcgataagggaacattagtgggtaaaaagaataaaatatttttagatgatgaaataaaagaaattgcagatttatatcattcatttaaatttttaaatgataatgattataaccatagtggtttttacaaaaaggttaacattgaaaaaatcgtggaaaatgattataaattaactccaactctctatgtaggtgtaaaggaagagactgaaatggagaagccatttagagaaatgataatagaatataaagcgatattagagcaacaatttgaagaatcaaacaaactacagcagaaaatattaaagaatttagagggattattatga

SEQ ID NO: 9 - protein, 511 aa, Bacillus stearothermophilus
  REGION 1..511: note = misc_feature - protein sequence for M.BstXI
MIFADIEFEKELFSAANKLRGKIAPSEYKHYVLPLIFLRYLSLKYQQRRNEIQQQINDSRDHKKNQDEVLKILEDRTEYTKVNVFYIPEKASWEYLLKNSENDKIKEMIDSAMEILENEYDELKGVLPKIYKNSNIPNEVISDLLKLFSQEVFSAHDGRNVDLLGRVYEYFISNFATTEGTRGGEYFTPSSIVKLLVAMLEPIKGTVYDPACGTGGMFIQSNKYRENNHNLCFVGQEQNELTIKLAKMNGILHGINPEIRQGDSLLNDRYPELKAEIVISNPPFNMKDWGAERLPLNDKRLIGPVTNSNANYMWIQHFLYHLKDGGLAGFVIANGALTSNLAAEKIVRKHLIDNDYVDCVVQLPEKMFFGTGIPSALVFLSKNRNGSNGHAKREKEVLFIDASDKGTLVGKKNKIFLDDEIKEIADLYHSFKFLNDNDYNHSGFYKKVNIEKIVENDYKLTPTLYVGVKEETEMEKPFREMIIEYKAILEQQFEESNKLQQKILKNLEGLL

SEQ ID NO: 10 - DNA, 1179 bp, Bacillus stearothermophilus
  misc_feature 1..1179: note = DNA sequence of S.BstXI
  source 1..1179: mol_type = other DNA
atgaaaagtactttgaaggaatataaattgggtgatattaccgaagtcgttaatggtgccactccttcaactaaaaagcctgagtactatgaaaatggtacaattccatggattactcctaaagatttatcaggctattactttaaatatatatctcatggtgaacgtaatataacagagcttggtctaagaaatagttcagctaagttgttaccaaaaggaactgtattattttcctcaagagccccaataggatacgtagcaatagctgataattggttaactacgaaccagggatttaaaagttttatatgtaatgaggagattatttacaatgaatacctttattattttcttattgctaaaagggattttattgaaacatttgcgaatgggagtacgtttaaagagctttcatcaacttctgcaaagaatataccaatcaatcttcctagtttagaagagcaaaagaagattgtgacaattttaggggatttggatagaaagatagaattaaattataaaattattgaaagcttagaaaaaatagcagaaagaacatataaatattggtttgtcgatgaattaaatcaagatgaacagcacatccgtaatggatgggaaactgctaaaattggcgatgtggtggaacttttgggagggggaacccctaaaacttcggaaagtaagtattgggaagatggagatattaattggtttactccttcagatttaacaaaaactagacagctttttgtacgtgattctcaaagaaaaataacaattgatggacttaataacagtgcagcgaaattaattcccccttattccttgttaatgtcaagtagagctacaattggcgagttggcaattaatcaagaatctgctactacaaatcaagggtttattgtattaataccaaatgaaaaaatttctatttaccaattatacttttgggctaaacttaataagagcaaaattatttcaatggcaaatggtagtacttttaaagaaattagtaagcgggattttaaatctttggagataatattaccaaaaaatatagacacttttaattcaattatgcaagattattttaggaaaattgaggagttaattgatgaaataaaaatcttaaaaaccgcaagagataatttaattccaaaacttataaaatga

SEQ ID NO: 11 - protein, 392 aa, Bacillus stearothermophilus
  REGION 1..392: note = misc_feature - protein sequence for S.BstXI
MKSTLKEYKLGDITEVVNGATPSTKKPEYYENGTIPWITPKDLSGYYFKYISHGERNITELGLRNSSAKLLPKGTVLFSSRAPIGYVAIADNWLTTNQGFKSFICNEEIIYNEYLYYFLIAKRDFIETFANGSTFKELSSTSAKNIPINLPSLEEQKKIVTILGDLDRKIELNYKIIESLEKIAERTYKYWFVDELNQDEQHIRNGWETAKIGDVVELLGGGTPKTSESKYWEDGDINWFTPSDLTKTRQLFVRDSQRKITIDGLNNSAAKLIPPYSLLMSSRATIGELAINQESATTNQGFIVLIPNEKISIYQLYFWAKLNKSKIISMANGSTFKEISKRDFKSLEIILPKNIDTFNSIMQDYFRKIEELIDEIKILKTARDNLIPKLIK

SEQ ID NO: 12 - DNA, 770 bp, Planococcus citreus
  source 1..770: mol_type = other DNA
atgaaacagtttgcagatccttttgaaagaagattccttgatgcaattgaacatcatcttgatggaatttctgagaaaataaaaaaagactttacacacaaaaactttttaaaagaattgaatggccttaaaggtgataaagtctatcatgacttaggctttgataccgctgaatatactctggtacgtcttataggaagaatgagcataagcgttgggagaaggctgggggagatatacgataaagtccctcgttatgttgctgccgcgcgatttggtcttcaaccaaatcaaattgcagaagtatttgatggtcttgagttagatatagctttgcgcaatagccttttgtcagatgatgataaaattcacataaaaaaaataactgaaaagatgtcaggcgaaacatactcgggaatcggaatcgaaattcgttataactttaatccaaatgacagttcccgtttaagaaaagacgtcgatgtagcttctaaattgtcggccgcggggttatttcctgtttatttaatatttagctctctcagtcctaggaatgatgcaatagcccgtcttaaaagagggggatggagctttaaacaggggcaggaagccttagacttccttaccgaacttttaggagtggatattgggtctgttttatctgacccaataatagccgcagaaactagggagaaaacatcaaaaattatgaagtctatatttgaatcagaggcattccaatctgttataccgggagagtggagtaaact

SEQ ID NO: 13 - protein, 257 aa, Planococcus citreus
MKQFADPFERRFLDAIEHHLDGISEKIKKDFTHKNFLKELNGLKGDKVYHDLGFDTAEYTLVRLIGRMSISVGRRLGEIYDKVPRYVAAARFGLQPNQIAEVFDGLELDIALRNSLLSDDDKIHIKKITEKMSGETYSGIGIEIRYNFNPNDSSRLRKDVDVASKLSAAGLFPVYLIFSSLSPRNDAIARLKRGGWSFKQGQEALDFLTELLGVDIGSVLSDPIIAAETREKTSKIMKSIFESEAFQSVIPGEWSKL

SEQ ID NO: 14 - DNA, 1347 bp, Planococcus citreus
  misc_feature 1..1347: note = DNA sequence for M.PciI
  source 1..1347: mol_type = other DNA
atgacaaatttttcgcactcagctctaacgagctacgatcttctcgggcatgaaattgtccaagattctgaagctgttagctcgggtccatatctggtcagctatgacccgatccctgtacgtcggtctacattcctagctggactgtcagagaacgttcactcgtggtttcgtctcacaccaagtttcggaccggatctagttcgaacaatcatcaaacagatgaatcttgcgccgcactcacacatccatgaccctttctcaggagccgggactaccgcgattgaggcttcgttagagggctatgaagcaagctgcgtagaagttaatccgtttctctacttcgtggggaaaacatccatagattggtctatcaatgctgatgatgctgcagcgcagctagaaagcattaaaaataaatattatagcatgtctgcaaccgctactttggataacatagccgacctaggaatagatataccaaaaatacacaatattcatcggtggtggagaaacgatgttcttaaagatatattagtcctaaaatcttctatcagatcttgcacacaagataagtattgttccttttttgagctagccctagctgcagttctcgttccagatttgacaaatgtaacgctaggaaaactacaactgcactttgtaaacaaagacgataaagagataaacgtctggcctacatatgaatctcatgcaaaaaaaatgattcacgacttgtcattaattaataagcaaaatttcgaatttttgcccaagattatttatggtgattcaactcaaaaatcaacatttagcgaggtggcagggatagatgctataataacatcccctccgtaccctaataggtacagctatatttggaatactcgccctcacctgtacattcttgatatgatttccgaagcaaaagaggcttcgcaaatagatcgtagaacgattggtggaacatgggggacagcaacttccgaattaggaaagggtatattttctccaatcaatgctgtagtcaaagacgcgcttgaaggggttcacgaaagaatcgccggttccgatcaactcatggcaaactatgtaactcattattttaatcggctctttttacatatagaagctataaaaccatcacttaatccaaaagcaaagcttgcttatgttgttgggaactcttggattaagggcgaatatgtagccactgacgtaatcttagcaaaaattatcgaaggggctttgccaggctcatcaattgatggtcttcatcgtttccgtcgccggaacagtggaaagaatctctttgaaactatagtttactccactctcccggtataa

SEQ ID NO: 15 - protein, 448 aa, Planococcus citreus
  REGION 1..448: note = misc_feature - Protein sequence for M.PciI
MTNFSHSALTSYDLLGHEIVQDSEAVSSGPYLVSYDPIPVRRSTFLAGLSENVHSWFRLTPSFGPDLVRTIIKQMNLAPHSHIHDPFSGAGTTAIEASLEGYEASCVEVNPFLYFVGKTSIDWSINADDAAAQLESIKNKYYSMSATATLDNIADLGIDIPKIHNIHRWWRNDVLKDILVLKSSIRSCTQDKYCSFFELALAAVLVPDLTNVTLGKLQLHFVNKDDKEINVWPTYESHAKKMIHDLSLINKQNFEFLPKIIYGDSTQKSTFSEVAGIDAIITSPPYPNRYSYIWNTRPHLYILDMISEAKEASQIDRRTIGGTWGTATSELGKGIFSPINAVVKDALEGVHERIAGSDQLMANYVTHYFNRLFLHIEAIKPSLNPKAKLAYVVGNSWIKGEYVATDVILAKIIEGALPGSSIDGLHRFRRRNSGKNLFETIVYSTLPV

SEQ ID NO: 16 - protein, 430 aa, Bacillus sphaericus
MRRLAKNSRNDSYLSNRDYQEIVRENTTTISFPLKEKHTLTLTKKIGLNQTAGFGGWFFPDSPCLLTVTVLSSFGTKVTSKTFSLSKDWNRVGLAWINEHSSDTMSIVLEFSDVEIVHTWGLTCDVFNVHELIIDAIEDQNKLIDVLNQEHLSPETYYLNHDSDTDLIENLESTEEIKIVNQSQKQISLKKCCYCQRYMPVNILVRSNSSFHKHKSKKTGFQNECRACKKWRINNSFNPVRTKDQLHESAVITREKKILLKEPEILQKIKNRNNGEGLKSIIWKKFDKKCFNCEKELTIEEVRLDHTRPLAYLWPIDEHATCLCEKCNNTKHDMFPIDFYQGDEDKLRRLARITGLDYESLVKRDVNEVELARIINNIEDFATNVEARTFRSIRNKVKEVRPDTDLFEILKSKNINLYNELQYELLTRKD

SEQ ID NO: 17 - DNA, 1245 bp, Enterobacter aerogenes
  source 1..1245: mol_type = other DNA
gtgaatcagaaaaatgaaaaatcatttatgcgtttgcaatcaacctttagcggtggcaaaggtagtccaatgcatgattggtacccatatttagagggttattctcccgaatttgtgaaatgcttgatttcacgatttgctcctaaagccaaaacaattttagatccattttgtggctctggaacaacagccattgtttccgttttagagggtttaaataattactattgcgaagtaaaccctttatgccaatatattattgaaactaaactaatagctttaacattaagcgaagaagaaaaaacaaaattagtaaatgaactttattctatttctaatgaaataactaatgtactcaaaccttctgcaaccgagacagatctagagaaatcatttaaatccgtttttggtaatacgaaattttttgaggatcacatatttaaagatatacttagttatcaatgttacattagctctatcgaagatgaaaatcttaagagacttctgacaatagcagggattagatcgttaatcccttcctcgttattggtaagacgaggtgatttacgattcaagacacaaaaagaattagagaaaggcaaccagggctttcgctttcatgtacaaaaaagcttagaattaattgccagtgatttattagacattacggaaggtagtggtttagctaccttcttatgtgatgatgccaaagaaatatctgggaataacctgattgatgctgtaataacaagcccgccatatttaaatggcacaaattattttagaaatactaaaattgaactttggtttatagggaaattaaagaccaaatcagatctaagacattatagggatttagctattaccagtggtattaacgatgtaactaaaggtaaaagcttatcttcaaataatactattatctcagaaataccattattatctgaatgtattaaagaactaagcataaaagagtatgatagtcgtatttcaatgatggttgaaaactacttttgggacatgttcaaattcttatcaaaactcccaaaattactaactaatgatgcgactatctgtatagatttaggtgattctgtttattgtaacgtctacatccctacacaagatattttgaaagaaatgatgtcaaagttaggttttgaagagaacgaaagggtcattcttcgtgaacgaaaatcccgcaatggaacaaagttagtccagactgttcaggtttttaaatga

SEQ ID NO: 18 - DNA, 1140 bp, Enterobacter aerogenes
  source 1..1140: mol_type = other DNA
atgaaaaataaatattttagtaaaaaatgggagcaattcaagaaagaattaccccatcaatcaggtgaaatggtaaagagaaattggggccataactggcactctatgtgttcataccaagggaaacttaaaccatcaatagctagatctttaattgatacattcatgccatcaagtaagggacgtatattagatgtcttctcaggtgttggcaccattcctttcgaagcaagattacttggtcatactgcatatggatttgatattagtccagcagcagttaatatttcacgcgcaaaactagaagttataagtaaaaatgaaatccaagaggtaattaataaattatctgattttattgagcaaaacaaaaattcaatagattataacgaacataatttaataaggtttaatggttcaattgaatcctattttcatcctgaaacttttaaggaaatactgtgtgctcgtaaattctttttaataaaaggtgaattaaatgcatctgaatcgttagtacagtcatgtttattacatattttacatggtaatcgtccgtatgcattgagtagaaagtcccatcctattacacctttcgcgcctactggagattttatatacagtaatttagttataaagttaatcaaaaaagttgaaagagtcttgcaaaattctgatggtatcccagatactggcagcaaagtattttatcaggactctacaaaaagttggcctgaagaagtaaataatttagatgcaattataacatcacctccattttatgatagtacccgtttctattcagcaaattggatgcgattatggttttctggttgggaaaaagatgacttccaaacgaagccaaaagattttgtggacgaaactcagaaaaaaagctttgaaatatatgataatatattcaaacaatctcaacaatgcttaaaaaaagatggcgtttttttaatgcacgttggcaaaagtaaaaaaagtgatatggcaggacaaattgctaaaattggtagtaattatcttagccttatagatatatttgacgaaagtgttgaacattgcgaaagtcacggaattaaagacaaaggcacgacaacccatcatcagtaccttgtctttacgaaagattag

SEQ ID NO: 19 - protein, 213 aa, Bacillus amyloliquefaciens
MEVEKEFITDEAKELLSKDKLIQQAYNEVKTSICSPIWPATSKTFTINNTEKNCNGVVPIKELCYTLLEDTYNWYREKPLDILKLEKKKGGPIDVYKEFIENSELKRVGMEFETGNISSAHRSMNKLLLGLKHGEIDLAIILMPIKQLAYYLTDRVTNFEELEPYFELTEGQPFIFIGFNAEAYNSNVPLIPKGSDGMSKRSIKKWKDKVENK

SEQ ID NO: 20 - protein, 194 aa, Oceanospirillum kriegii
MKIKRIEVLINNGSVPGIPMILNEIQDAIKTVSWPEGNNSFVINPVRKGNGVKPIKNSCMRHLHQKGWALEHPVRIKAEMRPGPLDAVKMIGGKAFALEWETGNISSSHRAINKMVMGMLERVIIGGVLILPSRDMYNYLTDRVGNFRELEPYFSVWRQFNLKDAYLAIVEIEHDSVDAQVSLIPKGTDGRAIR

SEQ ID NO: 21 - protein, 180 aa, Bacillus amyloliquefaciens
  REGION 1..180: note = MISC_FEATURE - Residues 1-180 correspond to residues 22-201 of the protein sequence of BamHI (seq id no. 19)
IQQAYNEVKTSICSPIWPATSKTFTINNTEKNCNGVVPIKELCYTLLEDTYNWYREKPLDILKLEKKKGGPIDVYKEFIENSELKRVGMEFETGNISSAHRSMNKLLLGLKHGEIDLAIILMPIKQLAYYLTDRVTNFEELEPYFELTEGQPFIFIGFNAEAYNSNVPLIPKGSDGMSKR

SEQ ID NO: 22 - protein, 177 aa, Oceanospirillum kriegii
  REGION 1..177: note = MISC_FEATURE - Residues 1-177 correspond to residues 18-194 of the protein sequence of OkrAI (seq id no. 20)
IPMILNEIQDAIKTVSWPEGNNSFVINPVRKGNGVKPIKNSCMRHLHQKGWALEHPVRIKAEMRPGPLDAVKMIGGKAFALEWETGNISSSHRAINKMVMGMLERVIIGGVLILPSRDMYNYLTDRVGNFRELEPYFSVWRQFNLKDAYLAIVEIEHDSVDAQVSLIPKGTDGRAIR

SEQ ID NO: 23 - DNA, 38 bp, synthetic construct
  misc_feature 1..38: note = primer
  source 1..38: mol_type = other DNA
attcaacaagcatacaatgcagttaaaacatctattgt

SEQ ID NO: 24 - DNA, 39 bp, synthetic construct
  misc_feature 1..39: note = primer
  source 1..39: mol_type = other DNA
acaaatagatgttttaactgcattgtatgcttgttgaat

SEQ ID NO: 25 - DNA, 39 bp, synthetic construct
  misc_feature 1..39: note = primer
  source 1..39: mol_type = other DNA
caagcatacaatgaagttgcaacatctatttgttcacct

SEQ ID NO: 26 - DNA, 39 bp, synthetic construct
  misc_feature 1..39: note = primer
  source 1..39: mol_type = other DNA
aggtgaacaaatagatgttgcaacttcattgtatgcttg

SEQ ID NO: 27 - DNA, 39 bp, synthetic construct
  misc_feature 1..39: note = primer
  source 1..39: mol_type = other DNA
acgattaacaacaccgaagcaaattgtaacggtgtagta

SEQ ID NO: 28 - DNA, 40 bp, synthetic construct
  misc_feature 1..40: note = primer
  source 1..40: mol_type = other DNA
acgattaacaacaccgaagcaaattgtaacggtgtagtat

SEQ ID NO: 29 - DNA, 39 bp, synthetic construct
  misc_feature 1..39: note = primer
  source 1..39: mol_type = other DNA
aacggtgtagtaccaattgcagaactatgttacacctta

SEQ ID NO: 30 - DNA, 39 bp, synthetic construct
  misc_feature 1..39: note = primer
  source 1..39: mol_type = other DNA
taaggtgtaacatagttctgcaattggtactacaccgtt

SEQ ID NO: 31 - DNA, 39 bp, synthetic construct
  misc_feature 1..39: note = primer
  source 1..39: mol_type = other DNA
aacccccttgatatacttgcacttgaaaagaaaaaaggt

SEQ ID NO: 32 - DNA, 39 bp, synthetic construct
  misc_feature 1..39: note = primer
  source 1..39: mol_type = other DNA
accttttttcttttcaagtgcaagtatatcaaggggttt

SEQ ID NO: 33 - DNA, 36 bp, synthetic construct
  misc_feature 1..36: note = primer
  source 1..36: mol_type = other DNA
gatatacttaaacttgcaaagaaaaaaggtggtccg

SEQ ID NO: 34 - DNA, 39 bp, synthetic construct
  misc_feature 1..39: note = primer
  source 1..39: mol_type = other DNA
cggaccaccttttttctttgcaagtttaagtatatcaag

SEQ ID NO: 35 - DNA, 39 bp, synthetic construct
  misc_feature 1..39: note = primer
  source 1..39: mol_type = other DNA
atacttaaacttgaaaaggcaaaaggtggtccgattgat

SEQ ID NO: 36 - DNA, 40 bp, synthetic construct
  misc_feature 1..40: note = primer
  source 1..40: mol_type = other DNA
atcaatcggaccaccttttgcctttttcaagtttaagtat

SEQ ID NO: 37 - DNA, 39 bp, synthetic construct
  misc_feature 1..39: note = primer
  source 1..39: mol_type = other DNA
ggtccgattgatgtttatgcagagttcatagaaaacagt

SEQ ID NO: 38 - DNA, 39 bp, synthetic construct
  misc_feature 1..39: note = primer
  source 1..39: mol_type = other DNA
actgttttctatgaactctgcataaacatcaatcggacc

SEQ ID NO: 39 - DNA, 37 bp, synthetic construct
  misc_feature 1..37: note = primer
  source 1..37: mol_type = other DNA
atagaaaaacagtgaacttgcacgtgtaggtatggaa

SEQ ID NO: 40 - DNA, 39 bp, synthetic construct
  misc_feature 1..39: note = primer
  source 1..39: mol_type = other DNA
aaattccatacctacacgtgcaagttcactgttttctat

SEQ ID NO: 41 - DNA, 39 bp, synthetic construct
  misc_feature 1..39: note = primer
  source 1..39: mol_type = other DNA
ggaaatattagttctgccgcacgttcaatgaacaaactt

SEQ ID NO: 42 - DNA, 39 bp, synthetic construct
  misc_feature 1..39: note = primer
  source 1..39: mol_type = other DNA
aagtttgttcattgaaacgtgcggcagaactaatattcc

SEQ ID NO: 43 - DNA, 39 bp, synthetic construct
  misc_feature 1..39: note = primer
  source 1..39: mol_type = other DNA
aatattagttctgcccacgcatcaatgaacaaacttcta

SEQ ID NO: 44 - DNA, 39 bp, synthetic construct
  misc_feature 1..39: note = primer
  source 1..39: mol_type = other DNA
tagaagtttgttcattgatgcgtgggcagaactaatatt

SEQ ID NO: 45 - DNA, 42 bp, synthetic construct
  misc_feature 1..42: note = primer
  source 1..42: mol_type = other DNA
gcccaccgttcaatgaacgcacttctattaggattaaaacat

SEQ ID NO: 46 - DNA, 42 bp, synthetic construct
  misc_feature 1..42: note = primer
  source 1..42: mol_type = other DNA
atgttttaatcctaatagaagtgcggtcattgaacggtgggc

SEQ ID NO: 47 - DNA, 39 bp, synthetic construct
  misc_feature 1..39: note = primer
  source 1..39: mol_type = other DNA
attatccttatgcctattgcacaattggcctattatctt

SEQ ID NO: 48 - DNA, 39 bp, synthetic construct
  misc_feature 1..39: note = primer
  source 1..39: mol_type = other DNA
aagataataggccaattgtgcaataggcataaggataat

SEQ ID NO: 49 - DNA, 39 bp, synthetic construct
  misc_feature 1..39: note = primer
  source 1..39: mol_type = other DNA
ttggcctattatcttacagcacgtgttaccaatttcgag

SEQ ID NO: 50 - DNA, 39 bp, synthetic construct
  misc_feature 1..39: note = primer
  source 1..39: mol_type = other DNA
ctcgaaattggtaacacgtgctgtaagataataggccaa

SEQ ID NO: 51 - DNA, 39 bp, synthetic construct
  misc_feature 1..39: note = primer
  source 1..39: mol_type = other DNA
gcctattatcttacagatgcagttaccaatttcgaggaa

SEQ ID NO: 52 - DNA, 39 bp, synthetic construct
  misc_feature 1..39: note = primer
  source 1..39: mol_type = other DNA
ttcctcgaaattggtaactgcatctgtaagataataggc

SEQ ID NO: 53 - DNA, 39 bp, synthetic construct
  misc_feature 1..39: note = primer
  source 1..39: mol_type = other DNA
cgtgttaccaatttcgaggcattagaaccttattttgaa

SEQ ID NO: 54 - DNA, 39 bp, synthetic construct
  misc_feature 1..39: note = primer
  source 1..39: mol_type = other DNA
ttcaaaataaggttctaatgcctcgaaattggtaacacg

SEQ ID NO: 55 - DNA, 39 bp, synthetic construct
  misc_feature 1..39: note = primer
  source 1..39: mol_type = other DNA
accaatttcgaggaattagcaccttattttgaacttact

SEQ ID NO: 56 - DNA, 39 bp, synthetic construct
  misc_feature 1..39: note = primer
  source 1..39: mol_type = other DNA
agtaagttcaaaataaggtgctaattcctcgaaattggt

SEQ ID NO: 57 - DNA, 42 bp, synthetic construct
  misc_feature 1..42: note = primer
  source 1..42: mol_type = other DNA
ccttattttgaacttactgcaggacaaccatttatttttatt

SEQ ID NO: 58 - DNA, 42 bp, synthetic construct
  misc_feature 1..42: note = primer
  source 1..42: mol_type = other DNA
aataaaaataaatggttgtgctgcagtaagttcaaaataagg

SEQ ID NO: 59 - DNA, 45 bp, synthetic construct
  misc_feature 1..45: note = primer
  source 1..45: mol_type = other DNA
tttatttttattggatttaatgctgcagcttataattctaatgtc

SEQ ID NO: 60 - DNA, 45 bp, synthetic construct
  misc_feature 1..45: note = primer
  source 1..45: mol_type = other DNA
gacattagaattataagctgcagcattaaatccaataaaaataaa

SEQ ID NO: 61 - DNA, 39 bp, synthetic construct
  misc_feature 1..39: note = primer
  source 1..39: mol_type = other DNA
aatgtccctttaattcccgcaggttctgacggtatgtca

SEQ ID NO: 62 - DNA, 39 bp, synthetic construct
  misc_feature 1..39: note = primer
  source 1..39: mol_type = other DNA
tgacataccgtcagaacctgcgggaattaaagggacatt

SEQ ID NO: 63 - DNA, 39 bp, synthetic construct
  misc_feature 1..39: note = primer
  source 1..39: mol_type = other DNA
ttaattcccaaaggttctgcaggtatgtcaaaacgctca

SEQ ID NO: 64 - DNA, 39 bp, synthetic construct
  misc_feature 1..39: note = primer
  source 1..39: mol_type = other DNA
tgagcgttttgacatacctgcagaacctttgggaattaa

SEQ ID NO: 65 - DNA, 39 bp, synthetic construct
  misc_feature 1..39: note = primer
  source 1..39: mol_type = other DNA
tctgacggtatgtcaaaagcatcaattaagaaatggaaa

SEQ ID NO: 66 - DNA, 39 bp, synthetic construct
  misc_feature 1..39: note = primer
  source 1..39: mol_type = other DNA
tttccatttcttaattgatgcttttgacataccgtcaga

SEQ ID NO: 67 - DNA, 54 bp, synthetic construct
  misc_feature 1..54: note = primer
  source 1..54: mol_type = other DNA
ggtggtgcatgcggaggtaaataaatggaagtagaaaaagagtttattactgat

SEQ ID NO: 68 - DNA, 51 bp, synthetic construct
  misc_feature 1..51: note = primer
  source 1..51: mol_type = other DNA
ggtggtggtaccctatttgttttcaactttatctttccatttcttaattga

SEQ ID NO: 69 - DNA, 39 bp, synthetic construct
  misc_feature 1..39: note = primer
  misc_feature 19..21: note = n=a, c, g or t
  source 1..39: mol_type = other DNA
caagcatacaatgaagttnnnacatctatttgttcacct

SEQ ID NO: 70 - DNA, 39 bp, synthetic construct
  misc_feature 1..39: note = primer
  misc_feature 19..21: note = n=a. c. g or t
  source 1..39: mol_type = other DNA
aggtgaacaaatagatgtnnnaacttcattgtatgcttg

SEQ ID NO: 71 - DNA, 37 bp, synthetic construct
  misc_feature 1..37: note = primer
  misc_feature 16..18
  misc_feature 16..18: note = n=a, c, g, or t
  source 1..37: mol_type = other DNA
gatatacttaaacttnnnaagaaaaaaaggtggtccg

SEQ ID NO: 72 - DNA, 39 bp, synthetic construct
  misc_feature 1..39: note = primer
  misc_feature 19..21
  misc_feature 19..21: note = n=a, c, g, or t
  source 1..39: mol_type = other DNA
cggaccaccttttttcttnnnaagtttaagtatatcaag

SEQ ID NO: 73 - DNA, 54 bp, synthetic construct
  misc_feature 1..54: note = primer
  source 1..54: mol_type = other DNA
ggtggtgcatgcggaggtaaataaatgtctaataaaaaacagtcaaataggcta

SEQ ID NO: 74 - DNA, 39 bp, synthetic construct
  misc_feature 1..39: note = primer
  source 1..39: mol_type = other DNA
ggtggtggtacctcacttagatctaagctgttcaaacaa

SEQ ID NO: 75 - protein, 267 aa, Escherichia coli
  REGION 1..267: note = MISC_FEATURE - Amino acid residues 1-267 correspond to residues 4-270 of the protein sequence of EcoRI
KKQSNRLTEQHKLSQGVIGIFGDYAKAHDLAVGEVSKLVKKALSNEYPQLSFRYRDSIKKTEINEALKKIDPDLGGTLFVSNSSIKPDGGIVEVKDDYGEWRVVLVAEAKHQGKDIINIRNGLLVGKRGDQDLMAAGNAIERSHKNISEIANFMLSESHFPYVLFLEGSNFLTENISITRPDGRVVNLEYNSGILNRLDRLTAANYGMPINSNLCINKFVNHKDKSIMLQAASIYTQGDGREWDSKIMFEIMFDISTTSLRVLGRDL

SEQ ID NO: 76 - protein, 265 aa, Rhodopseudomonas sphaeroides
  REGION 1..265: note = MISC_FEATURE - amino acid residues 1-265 correspond to residues 10-274 of the protein sequence of RsrI
KGQALRLGIQQELGGGPLSIFGAAAQKHDLSIREVTAGVLTKLAEDFPNLEFQLRTSLTKKAINEKLRSFDPRLGQALFVESASIRPDGGITEVKDRHGNWRVILVGESKHQGNDVEKILAGVLQGKAKDQDFMAAGNAIERMHKNVLELRNYMLDEKHFPYVVFLQGSNFATESFEVTRPDGRVVKIVHDSGMLNRIDRVTASSLSREINQNYCENIVVRAGSFDHMFQIASLYCKAAPWTAGEMAEAMLAVAKTSLRIIADDL

SEQ ID NO: 77 - DNA, 39 bp, synthetic construct
  misc_feature 1..39: note = primer
  source 1..39: mol_type = other DNA
gattgggtggcgcagaaatttcaaacgggccagcagtcg

SEQ ID NO: 78 - DNA, 39 bp, synthetic construct
  misc_feature 1..39: note = primer
  source 1..39: mol_type = other DNA
cgactgctggcccgtttgaaatttctgcgccacccaatc

SEQ ID NO: 79 - protein, 272 aa, Agrobacterium gelatinovorum
MRLDLDFGRGLVAHVMLDNVSEEQYQQISDYFVPLVNKPKLKSRDAIGQAFVMATEVCPDANPSDLWHHVLYRIYIREKIGTDPSQSWVRTSGEAFEVALVERYNPVLARHGIRLTALFKGQKGLALTRMGVADRVGSRKVDVMIEKQGGGRSPDAEGFGVVGGIHAKVSLAERVSDDIPASRIMMGEGLLSVLSTLDVKSFPPPHGDLVNRGELGTPDRPSDKRNYIEGHGDFSACFSYNLRTPPSNATTPSGRHIYVSASLVRTTSSPTT

SEQ ID NO: 80 - protein, 358 aa, Anabaena variabilis
MEEDLDLSENIEAASAELTTLYQVAADAMKDYIEIYLALSKQSDGFSNINNLDLTSRNRRLVVIHGLSLELDPDTSTPEEIKREAERMLAIALDTESAITAGVYEKMRLFASSLVDQLFEQTDELNSLSSEYLSANPGFLPFFQQLAGLRSKSELKREVGNASDNSISKAVAERILERIIRNLRIRTFSKEKLLQAVEPTLEGIVRDLVGKVLLENIVADALSDLQVPFMRESEYQSLKGVIYDFRADFVIPDAQNPIAFIEVRKSSTRHASLYAKDKMFSAINWKGKNKRLLGILVVEGPWTRETLRVMANVFDYVTPLTRVSQVAEAIRAYLDGDKTRLKWLVNFSIEEADHDNIT

SEQ ID NO: 81 - protein, 530 aa, Bacillus stearothermophilus
MAKYGRGKFLPHQNYIDYMHFIVNHKNYSGMPNAIGEDGRINWQVSSGKTTSFYEYYQARFEWWEKKADELNLPGTGNSNKRFSLAARLIHPTGQRPCRLCGKYQYVGYMYVSHNLYKRWSKITGREDLFFKKQNIIEAANIFKSIMGEQALINELTTIFPERKDYFNRLPNIEDFFVSSSHIKNNGNYISPGFMANPPDRLDGFHDYGICCRKEKDPGRHDDNMRLYNHDRRAFMWWSEGDWALADALYNKAGAGKCADPDCQKEVEKISPDHVGPISCGFKQIPFFKPLCASCNSAKNRRFSYQDVKELLKYENYTGDSVASWQVRALWDNCKHLVKNDDDSKLLSNLMRSLQDYYLRSLYKLFSNGFAHLLSYFLTPEYAHYKITFEGLNTSTLEYERYYKTFKKTKSTSSLAARIVRIAFEELEIYNSKDINERKLIKFDTSSWEKDFENIISYATKNLSLDEEASKWNKVLTDKNLSSTEKDKKISSLLEDKNYEVYKKQFYILKDLLVEHFNKIGEQIAKDYMK

SEQ ID NO: 82 - protein, 301 aa, Enterobacter agglomerans
MKKRRDLVEVFGYNPMDLSPEVRALWNLGACPFLNKECIKINHDQTIIYGTCSVTSPYGDVIICPNRLYANDYETLHKVSRDAFGDDVPFLTYSNFIKYRATYKDCIVALGKNSGKEVQVGRALSMDWVLVRITDGELKEYVGVEIQSIDITGNYRDAWHAYKNLKPIDIIDNLPTSQHGLNWANVHKRLIPQIIRKGVVYSRSNYVKKGLYFILPEIVYNKFEDVIGADIPLLKTQTNKSITVHTYSLGEPAANGEQRKLISEREIIFDLDEFSKRFTTGPNLPKGDDLDAVIKKALGMM

SEQ ID NO: 83 - protein, 277 aa, Escherichia coli
MSNKKQSNRLTEQHKLSQGVIGIFGDYAKAHDLAVGEVSKLVKKALSNEYPQLSFRYRDSIKKTEINEALKKIDPDLGGTLFVSNSSIKPDGGIVEVKDDYGEWRVVLVAEAKHQGKDIINIRNGLLVGKRGDQDLMAAGNAIERSHKNISEIANFMLSESHFPYVLFLEGSNFLTENISITRPDGRVVNLEYNSGILNRLDRLTAANYGMPINSNLCINKFVNHKDKSIMLQAASIYTQGDGREWDSKIMFEIMFDISTTSLRVLGRDLFEQLTSK

SEQ ID NO: 84 - protein, 245 aa, Escherichia coli
MSLRSDLINALYDENQKYDVCGIISAEGKIYPLGSDTKVLSTIFELFSRPIINKIAEKHGYIVEEPKQQNHYPDFTLYKPSEPNKKIAIDIKTTYTNKENEKIKFTLGGYTSFIRNNTKNIVYPFDQYIAHWIIGYVYTRVATRKSSLKTYNINELNEIPKPYKGVKVFLQDKWVIAGDLAGSGNTTNIGSIHAHYKDFVEGKGIFDSEDEFLDYWRNYERTSQLRNDKYNNISEYRNWIYRGRK

SEQ ID NO: 85 - protein, 300 aa, Haemophilus influenzae
MKKSALEKLLSLIENLTNQEFKQATNSLISFIYKLNRNEVIELVRSIGILPEAIKPSSTQEKLFSKAGDIVLAKAFQLLNLNSKPLEQRGNAGDVIALSKEFNYGLVADAKSFRLSRTAKNQKDFKVKALSEWREDKDYAVLTAPFFQYPTTKSQIFKQSLDENVLLFSWEHLAILLQLDLEETNIFPFEQLWNFPKKQSKKTSVSDAENNFMRDFNKYFMDLFKIDKDTLNQLLQKEINFIEERSLIEKEYWKKQINIIKNFTREEAIEALLKDINMSSKIETIDSFIKGIKSNDRLYL

SEQ ID NO: 86 - protein, 254 aa, Haemophilus parainfluenzae
MKYEEINFKVPVESPYYPNYSQCVIERIYSILRNQKDMGDDRIIINTNLKKGLPLENINKIAGPMIEAWAEEVFSGIRDNRDNQYNLINVEAQERLGISDIILQFQVNNNVITGNVDVKATSNDIPDSGKSPNITSFSRIRTAYVKDPNFIFIILSIKHSVYVKRNEYTNLMDGIMQIIDFNVYDLKYISDSDISYNPALGTGQIQIKDIHYVSSQKRTTWQMCQLLDLKYLRSKKRTIEQFYNEAKRNKWIKD

SEQ ID NO: 87 - protein, 218 aa, Klebsiella pnermoniae
MDVFDKVYSDDNNSYDQKTVSQRIEALFLNNLGKVVTRQQIIRAATDPKTGKQPENWHQRLSELRTDKGYTILSWRDMKVLAPQEYIMPHATRRPKAAKRVLPTKETWEQVLDRANYSCEWQEDGQHCGLVEGDIDPIGGGTVKLTPDHMTPHSIDPATDVNDPKMWQALCGRHQVMKKNYWDSNNGKINVIGILQSVNEKQKNDALEFLLNYYGLKR

SEQ ID NO: 88 - protein, 288 aa, Nocardia corallina
MATAPGHLLGQIIGNVMEEALKPVLQEMADRHDLYLDSKGLRPGVRSGALVTWTDDLGNNHDLDFVLERGGSATKAGNPAAFIEAAWRRYTKHSKAKAQEIQGAVLPVLAAWNNVKPTPAAVVAGQWTAPSLQQMRSNGFVVLHLHFPTTAQVFGGNGINIEGTGEGTPDAFWQQQCDAYTSKSEADKDSLATALRTAHAQEFRTFVAELERRVVRAIDYVVVTPLHGHGSQYTSIENAIEAVRTYSCGEESAPFLRFEIRISYTNGDVIQATFGSSSDAIEFLDTFN

SEQ ID NO: 89 - protein, 328 aa, Neisseria mucosa
MSSYHDDLNILNVDFNHLRLTELIKLADQAEPFYLWVEKIFRQVSGRADSLETIIEVEERVVLKMAILTCFTSDEKELPKLFNGVGVPYPHIKACYFFFAWLVRDAATQRLDPLIREAFTQLKSIHPQMKKTELESEIFSQLLVNYRNELIHFSWPVIREVLISRLEGSRRAARGSYLELFVRTALAQSITYFYKIYGNYGKFLDVKIHDKPLKVKNRTYDVVAELIGNNHNTQYLILPVKTRETQGGGHAHLFTRDIEQSNNDIRELYPNAVIAPVIIAENWSDTEKDLENVGYNDIFHFSVNPNRFAGFSDVEQIRLNRLVERILL

SEQ ID NO: 90 - protein, 383 aa, Nocardia otitidiscaviarum
MRSDTSVEPEGANFIAEFFGHRVYPEVVSTEAARNDQATGTCPFLTAAKLVETSCVKAETSRGVCVVNTAVDNERYDWLVCPNRALDPLFMSAASRKLFGYGPTEPLQFIAAPTLADQAVRDGIREWLDRGVHVVAYFQEKLGGELSISKTDSSPEFSFDWTLAEVESIYPVPKIKRYGVLEIQTMDFHGSYKHAVGAIDIALVEGIDFHGWLPTPAGRAALSKKMEGPNLSNVFKRTFYQMAYKFALSGHQRCAGTGFAIPQSVWKSWLRHLANPTLIDNGDGTFSLGDTRNDSENAWIFVFELDPDTDASPRPLAPHLEIRVNVDTLIDLALRESPRAALGPSGPVATFTDKVEARMLRFWPKTRRRRSTTPGGQRGLFDA

SEQ ID NO: 91 - protein, 326 aa, Providencia stuartii
MKELKLKEAKEILKALGLPPQQYNDRSGWVLLALANIKPEDSWKEAKAPLLPTVSIMEFIRTEYGKDYKPNSRETIRRQTLHQFEQARIVDRNRDLPSRATNSKDNNYSLNQVIIDILHNYPNGNWKELIQQFLTHVPSLQELYERALARDRIPIKLLDGTQISLSPGEHNQLHADIVHEFCPRFVGDMGKILYIGDTASSRNEGGKLMVLDSEYLKKLGVPPMSHDKLPDVVVYDEKRKWLFLIEAVTSHGPISPKRWLELEAALSSCTVGKVYVTAFPTRTEFRKNAANIAWETEVWIADNPDHMVHFNGDRFLGPHDKKPELS

SEQ ID NO: 92 - protein, 157 aa, Proteus vulgaris
MSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 93 - protein, 358 aa, Streptomyces achromogenes
MGITIKKSTAEQVLRKAYEAAASDDVFLEDWIFLATSLREVDAPRTYTAALVTALLARACDDRVDPRSIKEKYDDRAFSLRTLCHGVVVPMSVELGFDLGATGREPINNQPFFRYDQYSEIVRVQTKARPYLDRVSSALARVDEEDYSTEESFRALVAVLAVCISVANKKQRVAVGSAIVEASLIAETQSFVVSGHDVPRKLQACVAAGLDMVYSEVVSRRINDPSRDFPGDVQVILDGDPLLTVEVRGKSVSWEGLEQFVSSATYAGFRRVALMVDAASHVSLMSADDLTSALERKYECIVKVNESVSSFLRDVFVWSPRDVHSILSAFPEAMYRRMIEIEVREPELDRWAEIFPET

SEQ ID NO: 94 - protein, 315 aa, Streptomyces albus
MINADKPHRWNDDVQASVRLYNQWFLDAAPKAYRDTRQLTIDEVEQAFQRTANMTSITPEVLKAHPKTLATLRMSTAPPIARDRLVGLSHGSKSLLDTMEKGKLPPRMKGDVLDTHLAKMCAVLTDLLDLDLFHWYPTGEPAEPRQRELAATVVADRLCGAIADPIVRNAQERRQLALIEEWLLARGYTKKTHSASLPLNTMQPGTFSFRQNVVVGSDLPVNIPVDAVIQPHTPHSHKLPILIEAKSAGDFTNTNKRRKEEATKIHQLQLKYGNEISLTLFLCGYFNTGYLGYSAAEGLDWVWEHRIDDLEAAGA

SEQ ID NO: 95 - protein, 432 aa, Saccharopolyspora sp.
MRRLATQRREDAYKSNRDYQTVHEAQSLRVNSTDDDNLSLFLLKDISPREDSKNIVGFGGFVKPEIATTMALTLTTDIDKQIKSVPLSSNWNRISIVAKFASNPSVSITLGFDQTPWVDFWGINSDDIGLSFVSDAVPLEMSMIDSIHIAPETLYLDHSSACLLDIDPVESTRFKTGHGDPLSLKKCSYCGRLLPIDLERPGKLSFHKHRAKITNHQNECRSCKKWRINNSFNPMRTIDQLNESALITRERKIFLQEPEILQEIKDRTGAGLKSQVWERFHRKCFNCRKDLKLSEVQLDHTRPLAYLWPIDEHATCLCAQCNNTKKDRFPVDFYSEQQIRELSDICGLPYQDLCARSLNLDQLDRIERNIAEFSKEWDVRTFASTARRISEVYPARDLFETLKKESESAYNKIIEKLKERPDALLDEALPLD

SEQ ID NO: 96 - protein, 323 aa, Streptomyces sp.
MNSSDGIDGTVASIDTARALLKRFGFDAQRYNVRSAVTLLALAGLKPGDRWVDSTTPRLGVQKIMDWSGEHWAKPYATGSREDFRKKTLRQWVDNGFAVLNADNLNIATNSQLNEYCLSDEALQALRAYGTEGFEESLVVFLDEASKAVKARAEALQAAMISVDLPGGEEFLLSPAGQNPLLKKMVEEFVPRFAPRSTVLYLGDTRGKHSLFEREIFEEVLGLTFDPHGRMPDLILHDEVRGWLFLMEAVKSKGPFDEERHRSLQELFVTPSAGLIFVNCFENRESMRQWLPELAWETEAWVAEDPDHLIHLNGSRFLGPYER

SEQ ID NO: 97 - protein, 227 aa, Streptomyces caespitosus
MINDQLPRWVREARVGTRTGGPAMRPKTSDSPYFGWDSEDWPEVTRQLLSEQPLSGDTLVDAVLASWESIFESRLGSGFHIGTQIRPTPQIMGFLLHALIPLELANGDPSWRADLNSSEKDLVYQPDHKYSIEMKTSSHKDQIFGNRSFGVENPGKGKKAKDGYYVAVNFEKWSDAPGRLPRIRTIRYGWLDHTDWVAQKSQTGQQSSLPAVVSNTQLLAIHTGGQR

SEQ ID NO: 98 - protein, 235 aa, Streptomyces phaeochromogenes
MTSKDPIVLSADQIAWLRQLKMSKRAALVRDYILEYGAVTTGKLAELGYSHPPRAARDLKDAGAGVVTIMVKGPDGRRMASYAFNGKANEDGAGRVVIPKAFGEALKRAHGGKCAVCYGDFSERELQCDHRVPFAIAGDKPKLVQEDFMPLCASDNRAKSWSCENCPNWELKDEDTCRSCFWASPENYTHVSTRPERRINLLFQGDEVEIFDALKNAAANEGVSLTEATKRKLAD

SEQ ID NO: 99 - protein, 281 aa, Sphaerotilus sp.
MSKAAYQDFTKRFSLLIKKHPNLITMTLSNIFTMRLIGNKTHGDLAEIAISEFINQYMYDFKSIHVGKDLYRAKSKEEDITVENEITKEKFPISLKAYGDGPLQLSTDKNFLMYPLLEEIGAFINAKEKIEEIFANEAFSCFSEINVLPLIYDEKRQRCNILVFDAARARAETAYIRKETEGSGRKHPAYRFFDKNKNYICEVRYGNAAANALQRGLWTNTKNATSFFDSVTNGWVDYSHNLVLVKLLSHALVSSRKGHEAALEEIKKDILQLKQTNGINV

SEQ ID NO: 100 - DNA, 1077 bp, Anabaena variabilis
  source 1..1077: mol_type = other DNA
atggaagaagaccttgatttatctgaaaatatcgaagctgcatctgcggagcttacgactctttatcaggtagctgctgatgctatgaaagattatattgaaatctatcttgcgctgagtaaacagtctgatgggttttcaaatattaacaatcttgacttaacttctcgtaacaggcgtttggtagttatacatggactttcgttagagttagatccagatacttcgactccagaggaaattaaacgtgaagctgaacgaatgctagcgatagctcttgatacagagtcagcaattacggcaggagtatatgaaaaaatgcgtctcttcgcaagctctttagtagatcagctatttgaacaaacggatgaacttaattcattatcatcggaatatttgtcagcaaatccaggatttttgccgtttttccagcagttggcggggcttagaagtaaatcagagttaaagagagaagtaggaaatgcctctgacaatagtatttctaaagcggttgcagagagaatattagagcgcattatacgtaacttgagaattcgcactttttccaaagagaaactattacaagctgttgagcctactttagaaggaatagtcagggatctcgtaggaaaagtgttattggaaaatatagttgctgatgctttatctgatttacaagttcctttcatgcgtgaatcagagtatcaaagccttaaaggagtgatttatgatttccgcgctgattttgtgataccagacgcacaaaatccaattgcttttatcgaggtgcgaaaaagctctacacgacatgcgtcactctatgccaaggataagatgttttcagcgattaattggaaaggaaaaaataaaaggcttttgggtattttggttgtggaaggaccttggacaagagaaactcttcgcgtcatggcaaatgtgtttgattacgttacacctttaactcgtgtttcccaagttgcagaagctatcagagcatatctagatggggataaaacgagactgaagtggttagttaatttcagtattgaagaagcagaccacgacaacataacctaa

SEQ ID NO: 101 - DNA, 1293 bp, Bacillus sphaericus
  source 1..1293: mol_type = other DNA
atgagacgattagcaaaaaattcacggaacgacagttatttaagtaatagggattaccaggaaatcgtgagggaaaataccactacaatatcgtttcccttaaaagaaaaacatactctgactttaacgaaaaaaatagggctaaatcagactgctggattcggaggatggtttttccctgattcaccatgtttattaacagtaactgtactatcctctttcggtacaaaggtaacttctaaaacctttagcctttctaaagattggaatcgtgttgggcttgcttggattaacgagcattcgagtgacaccataagcattgtcctagagtttagtgatgtggaaatagttcatacatggggacttacatgtgatgtttttaatgtccatgaattaattattgatgctatagaagatcaaaataaactaatagacgtgctaaatcaagaacatttatctcctgaaacatattatttaaaccatgactctgatactgatttaattgagaatttggaatctacagaagagataaagatagttaaccaaagccaaaagcaaatctctttaaaaaaatgctgttattgtcaacgttatatgcctgtgaacatattagttcgttcaaattcatcatttcataaacacaagagtaagaaaactggttttcaaaatgaatgtcgggcttgtaagaagtggagaataaataattcattcaatccagtcagaacaaaagaccaactacatgaatcagcagttattacacgtgaaaaaaaaatattacttaaagaacctgaaatattacagaaaatcaaaaatagaaataacggtgagggcttaaaaagtattatatggaaaaaatttgataaaaaatgctttaattgtgaaaaagaattaaccattgaagaggtacgcctagaccatacaagaccacttgcttatctgtggcctatcgatgaacacgcaacttgtttatgtgaaaaatgcaacaatacaaaacatgatatgtttcctatcgatttttatcaaggggacgaagacaaattaagacgtttagctagaattacggggttagattatgaatctctagttaagagggacgtaaatgaagttgaacttgcaagaataatcaataacattgaagactttgcaactaatgtagaggcacgtacttttcgctcaataagaaataaagtaaaagaagtacgtcccgatactgacctatttgaaattcttaaatctaaaaatattaatttatataatgaacttcaatatgaacttcttacccgtaaggattaa

SEQ ID NO: 102 - DNA, 2189 bp, synthetic construct
  misc_feature 1..2189: note = plasmid placzz2
  source 1..2189: mol_type = other DNA
ctggcgaaagggggatgtgctgcaaggcgattaagttgggtaacgccagggttttcccagtcacgacgttgtaaaacgacggccagtgaattcgagctcggtacccgggggcgcgccggatccttaattaagtctagagtcgactgtttaaacctgcaggcatgcaagcttggcgtaatcatggtcatatgttaacctccggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 103 - DNA, 10673 bp, synthetic construct
  misc_feature 1..10673: note = plasmid pBC4
  source 1..10673: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgcggctgacgcggcggcagatggtgattacgaacccccgcggcgccgggcccggcactacctggacttggaggagggcgagggcctggcgcggctaggagcgccctctcctgagcgacacccaagggtgcagctgaagcgtgacacgcgcgaggcgtacgtgccgcggcagaacctgtttcgcgaccgcgagggagaggagcccgaggagatgcgggatcgaaagttccacgcagggcgcgagttgcggcatggcctgaaccgcgagcggttgctgcgcgaggaggactttgagcccgacgcgcggaccgggattagtcccgcgcgcgcacacgtggcggccgccgacctggtaaccgcgtacgagcagacggtgaaccaggagattaactttcaaaaaagctttaacaaccacgtgcgcacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacattctgcagagcatagtggtgcaggagcgcagcttgagcctggctgacaaggtggccgccattaactattccatgctcagtctgggcaagttttacgcccgcaagatataccataccccttacgttcccatagacaaggaggtaaagatcgaggggttctacatgcgcatggcgttgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgtaaccctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgatgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtccgggccagactattttttccagaccagtagacaaggcctgcagaccgtaaacctgagccaggctttcaagaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctgaactacctgctgaccaaccggcggcagaagatcccctcgttgcacagtttaaacagcgaggaggagcgcatcttgcgctatgtgcagcagagcgtgagccttaacctgatgcgcgacggggtaacgcccagcgtggcgctggacatgaccgcgcgcaacatggaaccgggcatgtatgcctcaaaccggccgtttatcaatcgcctaatggactacttgcatcgcgcggccgccgtgaaccccgagtatttcaccaatgccatcttgaacccgcactggctaccgccccctggtttctacaccgggggatttgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgcgagtagcccatttccaagcttgatagggtcttttaccagcactcgcaccacccgcccgcgcctgctgggcgaggaggagtacctaaacaactcgctgctgcagccgcagcgcgaaaagaacctgcctccggcatttcccaacaacgggatagagagcctagtggacaagatgagtagatggaagacgtatgcgcaggagcacagggatgtgcccggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaaaaaagcatgatgcaaaataaaaaactcaccaaggccatggcaccgagcgttggttttcttgtattccccttagtatgcagcgcgcggcgatgtatgaggaaggtcctcctccctcctacgagagcgtggtgagcgcggcgccagtggcggcggcgctgggttcccccttcgatgctcccctggacccgccgtttgtgcctccgcggtacctgcggcctaccggggggagaaacagcatccgttactctgagttggcacccctattcgacaccacccgtgtgtaccttgtggacaacaagtcaacggatgtggcatccctgaactaccagaacgaccacagcaactttctaaccacggtcattcaaaacaatgactacagcccgggggaggcaagcacacagaccatcaatcttgacgaccgttcgcactggggcggcgacctgaaaaccatcctgcataccaacatgccaaatgtgaacgagttcatgtttaccaataagtttaaggcgcgggtgatggtgtcgcgctcgcttactaaggacaaacaggtggagctgaaatatgagtgggtggagttcacgctgcccgagggcaactactccgagaccatgaccatagaccttatgaacaacgcgatcgtggagcactacttgaaagtgggcaggcagaacggggttctggaaagcgacatcggggtaaagtttgacacccgcaacttcagactggggtttgacccagtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgacctggagggtggtaacattcccgcactgttggatgtggacgcctaccaggcaagcttaaaagatgacaccgaacagggcggggatggcgcaggcggcggcaacaacagtggcagcggcgcggaagagaactccaacgcggcagccgcggcaatgcagccggtggaggacatgaacgatcatgccattcgcggcgacacctttgccacacgggcggaggagaagcgcgctgaggccgaggcagcggcagaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccgggatccgctcatggaccctcctttgcactcctgacgtaacctgcggctcggagcaggtctactggtcgttgccagacatgatgcaagaccccgtgaccttccgctccacgagccagatcagcaactttccggtggtgggcgccgagctgttgcccgtgcactccaagagcttctacaacgaccaggccgtctactcccagctcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaaacatgtccatccttatatcgcccagcaataacacaggctggggcctgcgcttcccaagcaagatgtttggcggggcaaagaagcgctccgaccaacacccagtgcgcgtgcgcgggcactaccgcgcgccctggggcgcgcacaaacgcggccgcactgggcgcaccaccgtcgatgacgccattgacgcggtggtggaggaggcgcgcaactacacgcccacgccgccaccagtgtccacagtggacgcggccattcagaccgtggtgcgcggagcccggcgttatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtactgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgatgaacttgacgacgaggtggaactgctgcacgcaaccgcgcccaggcggcgggtacagtggaaaggtcgacgcgtaagacgtgttttgcgacccggcaccaccgtagtttttacgcccggtgagcgctccacccgcacctacaagcgcgtgtatgatgaggtgtacggcgacgaggacctgcttgagcaggccaacgagcgcctcggggagtttgcctacggaaagcggcataaggacatgttggcgttgccgctggacgagggcaacccaacacctagcctaaagcccgtgacactgcagcaggtgctgcccacgcttgcaccgtccgaagaaaagcgcggcctaaagcgcgagtctggtgacttggcacccaccgtgcagctgatggtacccaagcgccagcgactggaagatgtcttggaaaaaatgaccgtggagcctgggctggagcccgaggtccgcgtgcggccaatcaagcaggtggcaccgggactgggcgtgcagaccgtggacgttcagatacccaccaccagtagcactagtattgccactgccacagagggcatggagacacaaacgtccccggttgcctcggcggtggcagatgccgcggtgcaggcggccgctgcggccgcgtccaaaacctctacggaggtgcaaacggacccgtggatgtttcgcgtttcagccccccggcgcccgcgccgttccaggaagtacggcaccgccagcgcactactgcccgaatatgccctacatccttccatcgcgcctacccccggctatcgtggctacacctaccgccccagaagacgagcgactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggagtctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccattaagaactatggcagcaaggcctggaacagcagcacaggccagatgctgagggacaagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcggcccgacagggaagaaactctggtgacgcaaatagatgagcctccctcgtacgaggaggcactaaagcaaggcctgcccaccacccgtcccatcgcgcccatggctaccggagtgctgggccagcacacacctgtaacgctggacctgcctccccccgctgacacccagcagaaacctgtgctgccagggccgtccgccgttgttgtaacccgccctagccgcgcgtccctgcgccgtgccgccagcggtccgcgatcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 104 - DNA, 22563 bp, synthetic construct
  misc_feature 1..22563: note = plasmid pXba
  source 1..22563: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatccttctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgcggctgacgcggcggcagatggtgattacgaacccccgcggcgccgggcccggcactacctggacttggaggagggcgagggcctggcgcggctaggagcgccctctcctgagcgacacccaagggtgcagctgaagcgtgacacgcgcgaggcgtacgtgccgcggcagaacctgtttcgcgaccgcgagggagaggagcccgaggagatgcgggatcgaaagttccacgcagggcgcgagttgcggcatggcctgaaccgcgagcggttgctgcgcgaggaggactttgagcccgacgcgcggaccgggattagtcccgcgcgcgcacacgtggcggccgccgacctggtaaccgcgtacgagcagacggtgaaccaggagattaactttcaaaaaagctttaacaaccacgtgcgcacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacattctgcagagcatagtggtgcaggagcgcagcttgagcctggctgacaaggtggccgccattaactattccatgctcagtctgggcaagttttacgcccgcaagatataccataccccttacgttcccatagacaaggaggtaaagatcgaggggttctacatgcgcatggcgttgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgtaaccctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgatgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtccgggccagactattttttccagaccagtagacaaggcctgcagaccgtaaacctgagccaggctttcaagaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctgaactacctgctgaccaaccggcggcagaagatcccctcgttgcacagtttaaacagcgaggaggagcgcatcttgcgctatgtgcagcagagcgtgagccttaacctgatgcgcgacggggtaacgcccagcgtggcgctggacatgaccgcgcgcaacatggaaccgggcatgtatgcctcaaaccggccgtttatcaatcgcctaatggactacttgcatcgcgcggccgccgtgaaccccgagtatttcaccaatgccatcttgaacccgcactggctaccgccccctggtttctacaccgggggatttgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgcgagtagcccatttccaagcttgatagggtcttttaccagcactcgcaccacccgcccgcgcctgctgggcgaggaggagtacctaaacaactcgctgctgcagccgcagcgcgaaaagaacctgcctccggcatttcccaacaacgggatagagagcctagtggacaagatgagtagatggaagacgtatgcgcaggagcacagggatgtgcccggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaaaaaagcatgatgcaaaataaaaaactcaccaaggccatggcaccgagcgttggttttcttgtattccccttagtatgcagcgcgcggcgatgtatgaggaaggtcctcctccctcctacgagagcgtggtgagcgcggcgccagtggcggcggcgctgggttcccccttcgatgctcccctggacccgccgtttgtgcctccgcggtacctgcggcctaccggggggagaaacagcatccgttactctgagttggcacccctattcgacaccacccgtgtgtaccttgtggacaacaagtcaacggatgtggcatccctgaactaccagaacgaccacagcaactttctaaccacggtcattcaaaacaatgactacagcccgggggaggcaagcacacagaccatcaatcttgacgaccgttcgcactggggcggcgacctgaaaaccatcctgcataccaacatgccaaatgtgaacgagttcatgtttaccaataagtttaaggcgcgggtgatggtgtcgcgctcgcttactaaggacaaacaggtggagctgaaatatgagtgggtggagttcacgctgcccgagggcaactactccgagaccatgaccatagaccttatgaacaacgcgatcgtggagcactacttgaaagtgggcaggcagaacggggttctggaaagcgacatcggggtaaagtttgacacccgcaacttcagactggggtttgacccagtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgacctggagggtggtaacattcccgcactgttggatgtggacgcctaccaggcaagcttaaaagatgacaccgaacagggcggggatggcgcaggcggcggcaacaacagtggcagcggcgcggaagagaactccaacgcggcagccgcggcaatgcagccggtggaggacatgaacgatcatgccattcgcggcgacacctttgccacacgggcggaggagaagcgcgctgaggccgaggcagcggcagaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccgggatccgctcatggaccctcctttgcactcctgacgtaacctgcggctcggagcaggtctactggtcgttgccagacatgatgcaagaccccgtgaccttccgctccacgagccagatcagcaactttccggtggtgggcgccgagctgttgcccgtgcactccaagagcttctacaacgaccaggccgtctactcccagctcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaaacatgtccatccttatatcgcccagcaataacacaggctggggcctgcgcttcccaagcaagatgtttggcggggcaaagaagcgctccgaccaacacccagtgcgcgtgcgcgggcactaccgcgcgccctggggcgcgcacaaacgcggccgcactgggcgcaccaccgtcgatgacgccattgacgcggtggtggaggaggcgcgcaactacacgcccacgccgccaccagtgtccacagtggacgcggccattcagaccgtggtgcgcggagcccggcgttatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtactgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgatgaacttgacgacgaggtggaactgctgcacgcaaccgcgcccaggcggcgggtacagtggaaaggtcgacgcgtaagacgtgttttgcgacccggcaccaccgtagtttttacgcccggtgagcgctccacccgcacctacaagcgcgtgtatgatgaggtgtacggcgacgaggacctgcttgagcaggccaacgagcgcctcggggagtttgcctacggaaagcggcataaggacatgttggcgttgccgctggacgagggcaacccaacacctagcctaaagcccgtgacactgcagcaggtgctgcccacgcttgcaccgtccgaagaaaagcgcggcctaaagcgcgagtctggtgacttggcacccaccgtgcagctgatggtacccaagcgccagcgactggaagatgtcttggaaaaaatgaccgtggagcctgggctggagcccgaggtccgcgtgcggccaatcaagcaggtggcaccgggactgggcgtgcagaccgtggacgttcagatacccaccaccagtagcactagtattgccactgccacagagggcatggagacacaaacgtccccggttgcctcggcggtggcagatgccgcggtgcaggcggccgctgcggccgcgtccaaaacctctacggaggtgcaaacggacccgtggatgtttcgcgtttcagccccccggcgcccgcgccgttccaggaagtacggcaccgccagcgcactactgcccgaatatgccctacatccttccatcgcgcctacccccggctatcgtggctacacctaccgccccagaagacgagcgactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggagtctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccattaagaactatggcagcaaggcctggaacagcagcacaggccagatgctgagggacaagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcggcccgacagggaagaaactctggtgacgcaaatagatgagcctccctcgtacgaggaggcactaaagcaaggcctgcccaccacccgtcccatcgcgcccatggctaccggagtgctgggccagcacacacctgtaacgctggacctgcctccccccgctgacacccagcagaaacctgtgctgccagggccgtccgccgttgttgtaacccgccctagccgcgcgtccctgcgccgtgccgccagcggtccgcgatcgatgcggcccgtagccagtggcaactggcaaagcacactgaacagcatcgtgggtctgggggtgcaatccctgaagcgccgacgatgcttctaaatagctaacgtgtcgtatgtgtcatgtatgcgtccatgtcgccgccagaggagctgctgagccgccgtgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcacctacgcacgacgtaaccacagaccggtcccagcgtttgacgctgcggttcatccctgtggaccgcgaggataccgcgtactcgtacaaagcgcggttcaccctggctgtgggtgacaaccgtgtgcttgatatggcttccacgtactttgacatccgcggcgtgctggacagggggcctacttttaagccctactccggcactgcctacaacgctctagctcccaagggcgctcctaactcctgtgagtgggaacaaaccgaagatagcggccgggcagttgccgaggatgaagaagaggaagatgaagatgaagaagaggaagaagaagagcaaaacgctcgagatcaggctactaagaaaacacatgtctatgcccaggctcctttgtctggagaaacaattacaaaaagcgggctacaaataggatcagacaatgcagaaacacaagctaaacctgtatacgcagatccttcctatcaaccagaacctcaaattggcgaatctcagtggaacgaagctgatgctaatgcggcaggagggagagtgcttaaaaaaacaactcccatgaaaccatgctatggatcttatgccaggcctacaaatccttttggtggtcaatccgttctggttccggatgaaaaaggggtgcctcttccaaaggttgacttgcaattcttctcaaatactacctctttgaacgaccggcaaggcaatgctactaaaccaaaagtggttttgtacagtgaagatgtaaatatggaaaccccagacacacatctgtcttacaaacctggaaaaggtgatgaaaattctaaagctatgttgggtcaacaatctatgccaaacagacccaattacattgctttcagggacaattttattggcctaatgtattataacagcactggcaacatgggtgttcttgctggtcaggcatcgcagctaaatgccgtggtagatttgcaagacagaaacacagagctgtcctatcaactcttgcttgattccataggtgatagaaccagatatttttctatgtggaatcaggctgtagacagctatgatccagatgttagaatcattgaaaaccatggaactgaggatgaattgccaaattattgttttcctcttgggggtattggggtaactgacacctatcaagctattaaggctaatggcaatggctcaggcgataatggagatactacatggacaaaagatgaaacttttgcaacacgtaatgaaataggagtgggtaacaactttgccatggaaattaacctaaatgccaacctatggagaaatttcctttactccaatattgcgctgtacctgccagacaagctaaaatacaaccccaccaatgtggaaatatctgacaaccccaacacctacgactacatgaacaagcgagtggtggctcccgggcttgtagactgctacattaaccttggggcgcgctggtctctggactacatggacaacgttaatccctttaaccaccaccgcaatgcgggcctccgttatcgctccatgttgttgggaaacggccgctacgtgccctttcacattcaggtgccccaaaagttttttgccattaaaaacctcctcctcctgccaggctcatatacatatgaatggaacttcaggaaggatgttaacatggttctgcagagctctctgggaaacgatcttagagttgacggggctagcattaagtttgacagcatttgtctttacgccaccttcttccccatggcccacaacacggcctccacgctggaagccatgctcagaaatgacaccaacgaccagtcctttaatgactacctttccgccgccaacatgctataccccatacccgccaacgccaccaacgtgcccatctccatcccatcgcgcaactgggcagcatttcgcggttgggccttcacacgcttgaagacaaaggaaaccccttccctgggatcaggctacgacccttactacacctactctggctccataccataccttgacggaaccttctatcttaatcacacctttaagaaggtggccattacctttgactcttctgttagctggccgggcaacgaccgcctgcttactcccaatgagtttgagattaaacgctcagttgacggggagggctacaacgtagctcagtgcaacatgaccaaggactggttcctggtgcagatgttggccaactacaatattggctaccagggcttctacattccagaaagctacaaggaccgcatgtactcgttcttcagaaacttccagcccatgagccggcaagtggttgacgatactaaatacaaggagtatcagcaggttggaattcttcaccagcataacaactcaggattcgtaggctacctcgctcccaccatgcgcgagggacaggcttaccccgccaacgtgccctacccactaataggcaaaaccgcggttgacagtattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgagaccgtgtacctgcgcacgcccttctcggccggcaacgccacaacataaaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcgctcaaaaacatgctacctctttgagccctttggcttttctgaccaacgactcaagcaggtttaccagtttgagtacgagtcactcctgcgccgtagcgccattgcttcttcccccgaccgctgtataacgctggaaaagtccacccaaagcgtgcaggggcccaactcggccgcctgtggactattctgctgcatgtttctccacgcctttgccaactggccccaaactcccatggatcacaaccccaccatgaaccttattaccggggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgaaaatcagagggcgaggtattcagctcaacgacgagtcggtgagctcctctcttggtctccgtccggacgggacatttcagatcggcggcgctggccgctcttcatttacgccccgtcaggcgatcctaactctgcagacctcgtcctcggagccgcgctccggaggcattggaactctacaatttattgaggagttcgtgccttcggtttacttcaaccccttttctggacctcccggccactacccggaccagtttattcccaactttgacgcggtgaaagactcggcggacggctacgactgaatgaccagtggagaggcagagcgactgcgcctgacacacctcgaccactgccgccgccacaagtgctttgcccgcggctccggtgagttttgttactttgaattgcccgaagagcatatcgagggcccggcgcacggcgtccggctcaccacccaggtagagcttacacgtagcctgattcgggagtttaccaagcgccccctgctagtggagcgggagcggggtccctgtgttctgaccgtggtttgcaactgtcctaaccctggattacatcaagatctttgttgtcatctctgtgctgagtataataaatacagaaattagaatctactggggctcctgtcgccatcctgtgaacgccaccgtttttacccacccaaagcagaccaaagcaaacctcacctccggtttgcacaagcgggccaataagtaccttacctggtactttaacggctcttcatttgtaatttacaacagtttccagcgagacgaagtaagtttgccacacaaccttctcggcttcaactacaccgtcaagaaaaacaccaccaccaccaccctcctcacctgccgggaacgtacgagtgcgtcaccggttgctgcgcccacacctacagcctgagcgtaaccagacattactcccatttttccaaaacaggaggtgagctcaactcccggaactcaggtcaaaaaagcattttgcggggtgctgggattttttaattaagtatatgagcaattcaagtaactctacaagcttgtctaatttttctggaattggggtcggggttatccttactcttgtaattctgtttattcttatactagcacttctgtgccttagggttgccgcctgctgcacgcacgtttgtacctattgtcagctttttaaacgctgggggcaacatccaagatgaggtacatgattttaggcttgctcgcccttgcggcagtctgcagcgctgccaaaaaggttgagtttaaggaaccagcttgcaatgttacatttaaatcagaagctaatgaatgcactactcttataaaatgcaccacagaacatgaaaagcttattattcgccacaaagacaaaattggcaagtatgctgtatatgctatttggcagccaggtgacactaacgactataatgtcacagtcttccaaggtgaaaatcgtaaaacttttatgtataaatttccattttatgaaatgtgcgatattaccatgtacatgagcaaacagtacaagttgtggcccccacaaaagtgtttagagaacactggcaccttttgttccaccgctctgcttattacagcgcttgctttggtatgtaccttactttatctcaaatacaaaagcagacgcagttttattgatgaaaagaaaatgccttgattttccgcttgcttgtattcccctggacaatttactctatgtgggatatgctccaggcgggcaagattatacccacaaccttcaaatcaaactttcctggacgttagcgcctgatttctgccagcgcctgcactgcaaatttgatcaaacccagcttcagcttgcctgctccagagatgaccggctcaaccatcgcgcccacaacggactatcgcaacaccactgctaccggactaacatctgccctaaatttaccccaagttcatgcctttgtcaatgactgggcgagcttggacatgtggtggttttccatagcgcttatgtttgtttgccttattattatgtggcttatttgttgcctaaagcgcagacgcgccagaccccccatctataggcctatcattgtgctcaacccacacaatgaaaaaattcatagattggacggtctgaaaccatgttctcttcttttacagtatgattaaatgagacatgattcctcgagttcttatattattgacccttgttgcgcttttctgtgcgtgctctacattggccgcggtcgctcacatcgaagtagattgcatcccacctttcacagtttacctgctttacggatttgtcacccttatcctcatctgcagcctcgtcactgtagtcatcgccttcattcagttcattgactgggtttgtgtgcgcattgcgtacctcaggcaccatccgcaatacagagacaggactatagctgatcttctcagaattctttaattatgaaacggagtgtcatttttgttttgctgattttttgcgccctacctgtgctttgctcccaaacctcagcgcctcccaaaagacatatttcctgcagattcactcaaatatggaacattcccagctgctacaacaaacagagcgatttgtcagaagcctggttatacgccatcatctctgtcatggttttttgcagtaccatttttgccctagccatatatccataccttgacattggctggaatgccatagatgccatgaaccaccctactttcccagtgcccgctgtcataccactgcaacaggttattgccccaatcaatcagcctcgccccccttctcccacccccactgagattagctactttaatttgacaggtggagatgactgaatctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 105 - DNA, 8350 bp, synthetic construct
  misc_feature 1..8350: note = plasmid psyx20-lacIq
  source 1..8350: mol_type = other DNA
cagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccgcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaaccggctgaagccggatctgcgattctgataacaaactagcaacaccagaacagcccgtttgcgggcagcaaaacccgtacttttggacgttccggcggttttttgtggcgagtggtgttcgggcggtgcgcgattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgcgcgcgaaggccaagcggcatgcatttacgttgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcgaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggaatcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgc

SEQ ID NO: 106 - DNA, 3190 bp, synthetic construct
  misc_feature 1..3190: note = plasmid pAGR3
  source 1..3190: mol_type = other DNA
aagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattcccaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattggggatcg

(19) United States
(12) Patent Application Publication
(10) Pub. No.: US 2024/0352447 A1
(43) Pub. Date: Oct. 24, 2024

(54) High Fidelity Restriction Endonucleases

(71) Applicant: New England Biolabs, Inc., Ipswich, MA (US)

(72) Inventors: Zhenyu Zhu, Beverly, MA (US); Aine Quimby, Newton, NH (US); Shuang-Yong Xu, Lexington, MA (US); Shengxi Guan, Stoneham, MA (US); Hua Wei, Ipswich, MA (US); Penghua Zhang, Lexington, MA (US); Dapeng Sun, Arlington, MA (US); Siu-hong Chan, Ipswich, MA (US)

(73) Assignee: New England Biolabs, Inc., Ipswich, MA (US)

(21) Appl. No.: 18/750,621

(22) Filed: Jun. 21, 2024

Related U.S. Application Data
(62) Division of application No. 17/227,799, filed on Apr. 12, 2021, now Pat. No. 12,054,707.
Application No. 17/227,799 is a division of application No. 16/567,332, filed on Sep. 11, 2019, now abandoned.
Application No. 16/567,332 is a division of application No. 15/882,603, filed on Jan. 29, 2018, now Pat. No. 10,450,559.
Application No. 15/882,603 is a division of application No. 14/972,844, filed on Dec. 17, 2015, now abandoned.
Application No. 14/972,844 is a division of application No. 13/736,406, filed on Jan. 8, 2013, now Pat. No. 9,249,396.
Application No. 13/736,406 is a division of application No. 12/172,963, filed on Jul. 14, 2008, now Pat. No. 8,372,619.
(60) Provisional application No. 60/959,203, filed on Jul. 12, 2007.

(51) Int. Cl. C12N 15/10; C12N 9/16; C12N 9/22

(52) U.S. Cl. CPC C12N 15/1058; C12N 9/16; C12N 9/22; C12N 15/1034; C12N 15/1086

(57) ABSTRACT

Compositions and methods are provided for enzymes with altered properties that involve a systematic approach to mutagenesis and a screening assay that permits selection of the desired proteins. Embodiments of the method are particularly suited for modifying specific properties of restriction endonucleases such as star activity. The compositions includes restriction endonucleases with reduced star activity as defined by an overall fidelity index improvement factor.

### BACKGROUND

Restriction endonucleases are enzymes that cleave double-stranded DNAs in a sequence-specific manner (Roberts, R. J., Proc Natl Acad Sci USA, 102:5905-5908 (2005); Roberts, et al., Nucleic Acids Res, 31:1805-1812 (2003); Roberts, et al., Nucleic Acids Res, 33:D230-232 (2005); Alves, et al., Restriction Endonucleases, “Protein Engineering of Restriction Enzymes,” ed. Pingoud, Springer-Verlag Berlin Heidelberg, New York, 393-407 (2004)). They are ubiquitously present among prokaryotic organisms (Raleigh, et al., Bacterial Genomes Physical Structure and Analysis, Ch.8, eds. De Bruijin, et al., Chapman & Hall, New York, 78-92 (1998)), in which they form part of restriction-modification systems, which mainly consist of an endonuclease and a methyltransferase. The cognate methyltransferase methylates the same specific sequence that its paired endonuclease recognizes and renders the modified DNA resistant to cleavage by the endonuclease so that the host DNA can be properly protected. However, when there is an invasion of foreign DNA, in particular bacteriophage DNA, the foreign DNA will be degraded before it can be completely methylated. The major biological function of the restriction-modification system is to protect the host from bacteriophage infection (Arber, Science, 205:361-365 (1979)). Other functions have also been suggested, such as involvement in recombination and transposition (Carlson, et al., Mol Microbiol, 27:671-676 (1998); Heitman, Genet Eng (NY), 15:57-108 (1993); McKane, et al., Genetics, 139:35-43 (1995)).

The specificity of the approximately 3,000 known restriction endonucleases for their greater than 250 different target sequences could be considered their most interesting characteristic. After the discovery of the sequence-specific nature of the first restriction endonuclease (Danna, et al., Proc Natl Acad Sci USA, 68:2913-2917 (1971); Kelly, et al., J Mol Biol, 51:393-409 (1970)), it did not take long for scientists to find that certain restriction endonucleases cleave sequences which are similar but not identical to their defined recognition sequences under non-optimal conditions (Polisky, et al., Proc Natl Acad Sci USA , 72:3310-3314 (1975); Nasri, et al., Nucleic Acids Res, 14:811-821 (1986)). This relaxed specificity is referred to as star activity of the restriction endonuclease. It has been suggested that water-mediated interactions between the restriction endonuclease and DNA are the key differences between specific complexes and star complexes (Robinson, et al., J Mol Biol, 234:302-306 (1993); Robinson, et al., Proc Natl Acad Sci USA, 92:3444-3448 (1995), Sidorova, et al., Biophys J, 87:2564-2576 (2004)).

Star activity is a problem in molecular biology reactions. Star activity introduces undesirable cuts in a cloning vector or other DNA. In cases such as forensic applications, where a certain DNA substrate needs to be cleaved by a restriction endonuclease to generate a unique fingerprint, star activity will alter a cleavage pattern profile, thereby complicating analysis. Avoiding star activity is also critical in applications such as strand displacement amplification (Walker, et al., Proc Natl Acad Sci USA, 89:392-396 (1992)) and serial analysis of gene expression (Velculescu, et al., Science, 270:484-487 (1995)).

### SUMMARY

In an embodiment of the invention, a composition is provided that includes a restriction endonuclease having at least one artificially introduced mutation and an overall fidelity index (FI) improvement factor of at least two, the restriction endonuclease being capable of cleaving a substrate with at least a similar cleavage activity to that of the restriction endonuclease absent the artificially introduced mutation in a predetermined buffer, the artificially introduced mutation being the product of at least one of a targeted mutation, saturation mutagenesis, or a mutation introduced through a PCR amplification procedure.

In a further embodiment of the invention, at least one of the artificially introduced mutations is a targeted mutation resulting from replacement of a naturally occurring residue with an oppositely charged residue. An Alanine or a Phenylalanine may replace the naturally occurring residue at the target site.

In a further embodiment of the invention, a composition of the type described above includes a restriction enzyme absent the artificially introduced mutation selected from the group consisting of: BamHI, EcoRI, ScaI, SalI, SphI, PstI, NcoI, NheI, SspI, NotI, SacI, PvuII, MfeI, HindIII, SbfI, EagI, EcoRV, AvrII, BstXI, PciI, HpaI, AgeI, BsmBI, BspQI, SapI, KpnI and BsaI.

Further embodiments of the invention include compositions listed in Table 4.

In a further embodiment of the invention, a DNA encoding any of the enzymes listed in Table 4 is provided, a vector comprising the DNA and a host cell for expressing the protein from the vector.

In an embodiment of the invention, a method is provided having the steps of (a) identifying which amino acid residues in an amino acid sequence of a restriction endonuclease having star activity are charged amino acids; (b) mutating one or more codons encoding one or more of the charged residues in a gene sequence encoding the restriction endonuclease; (c) generating a library of gene sequences having one or more different codon mutations in different charged residues; (d) obtaining a set of proteins expressed by the mutated gene sequences; and (e) determining an FI in a predetermined buffer and a cleavage activity for each expressed protein.

An embodiment of the method includes the step of determining an overall FI improvement factor for proteins belonging to the set of proteins in a defined set of buffers where for example, the set of buffers contains NEB1, NEB2, NEB3 and NEB4 buffers.

An embodiment of the method includes the steps described above and additionally mutating codons encoding hydroxylated amino acids or amide amino acids in a same or subsequent step to that of mutating codons for the charged amino acids.

In an embodiment of the invention described above, the codons are mutated to an Alanine except for Tyrosine which is mutated to a Phenylalanine.

In a further embodiment, the overall FI improvement factor is improved using saturation mutagenesis of one or more of the mutated codon.

### BRIEF DESCRIPTION OF THE DRAWINGS

For FIGS. 1-32:

The * symbol indicates the lane to its left that contains the lowest concentration of enzyme for which star activity is observed.

The #symbol refers to the lane showing incomplete cleavage, which is adjacent to and to the right side of the lane containing a concentration of enzyme sufficient for complete cleavage of the substrate.

The gray triangle denotes the serial decrease of restriction endonuclease concentration.

“U” denotes units of enzyme.

In each of the reactions described in FIGS. 1-32, the reaction mixture contains a volume of 3 μl unless otherwise specified of a buffer from New England Biolabs, Inc. (NEB), Ipswich, MA, (see Table 1 and NEB catalog), 3 μl unless otherwise specified of a specified restriction endonuclease in a diluent from NEB, Ipswich, MA (See Table 1 and NEB catalog) as well as variable volumes of specified substrate (containing 0.6 μg) substrate and a volume of water to bring the reaction mixture to a total of 30 μl. Reactions were conducted at 37° C. for an incubation time of 1 hour. The results are analyzed on a 0.8% agarose gel. Where the overall volume of the reaction mix, amount of substrate, temperature of the reaction or incubation time varies from above, values are provided in the description of the figures.

The theoretical digestion pattern is provided on the right side of the gel for FIGS. 1, 5, 8, 11-18 and 20-32. Those substrates with only one restriction endonuclease site should be digested into one linear band from supercoiled form.

FIG. 1 shows the determination of the FI for wild type (WT) ScaI by digesting 1.2 μl lambda DNA substrate (0.6 μg) with a two-fold serial dilution using diluent A of a preparation of WT ScaI (1,200 U) in NEB3 buffer and examining the digestion products on an agarose gel. The highest concentration of a restriction endonuclease with no star activity is shown with a solid arrow; and the minimum concentration giving rise to complete digestion of substrate is shown with a hollow arrow.

FIGS. 2A-D show the results of digesting 0.5 μl pUC19 substrate (0.5 μg) with WT BamHI or BamHI(E86P) enzyme in a three-fold serial dilution using diluent A for 1 hour at a starting concentration of 172 U or 512 U. The middle lane is the NEB 1 kb marker (New England Biolabs, Inc. (NEB), Ipswich, MA).

FIG. 2A shows results using NEB1 buffer.

FIG. 2B shows results using NEB2 buffer.

FIG. 2C shows results using NEB3 buffer.

FIG. 2D shows results using NEB4 buffer.

FIGS. 3A-B show a comparison of BamHI(E86P) activity over two time periods using 0.6 μl pBR322 substrate (which contains only 1 BamHI cleavage site) in NEB2 buffer using an initial concentration of 600 U of enzyme in a 2-fold serial dilution using diluent A.

FIG. 3A shows results in 1 hour.

FIG. 3B shows results in 14 hours.

FIGS. 4A-B show the cleavage of 0.6 μl pBR322 substrate in a 2-fold serial dilution of BamHI-HF (E163A/E167T) using diluent A after 14 hours incubation in two different buffers on an agarose gel.

FIG. 4A shows the results with NEB2 buffer with an initial concentration of 600 U of enzyme.

FIG. 4B shows the results with NEB1 buffer with an initial concentration of 2,400 U of enzyme.

FIGS. 5A-B show a comparison of cleavage reactions using BamHI-HF and WT BamHI in NEB4 buffer. The reaction was carried out in NEB4 buffer using 1.2 μl lambda DNA substrate in a 2-fold serial dilution using diluent A.

FIG. 5A shows WT BamHI with a starting concentration of 1,200 U where the FI equals 4.

FIG. 5B shows BamHI-HF with a starting concentration of 2,400 U where the FI≥4000.

FIGS. 6A-D show a comparison of WT EcoRI and EcoRI(K62A) in NEB1-4 buffers in a 3-fold serial dilution using NEB diluent C. The reaction mixture contained 2 μl lambda DNA substrate (1 μg) in NEB1-4 buffers.

FIG. 6A shows the cleavage results following 2-fold serial dilution, 120 U WT EcoRI and 240 U of EcoRI (K62A) in NEB2 buffer.

FIG. 6B shows the cleavage results following 2-fold serial dilution, 120 U WT EcoRI and 240 U of EcoRI (K62A) in NEB4 buffer.

FIG. 6C shows the cleavage results following 2-fold serial dilution, 60 U WT EcoRI and 120 U of EcoRI (K62A) in NEB1 buffer.

FIG. 6D shows the cleavage results following 2-fold serial dilution, 120 U WT EcoRI and 60 U of EcoRI (K62A) in NEB3 buffer.

FIGS. 7A-C show the cleavage results with 2 different EcoRI mutants and WT EcoRI. The digestion of 100,000 U of enzyme and a 10-fold serial dilution thereof in diluent C over 10 hours using 0.6 μl of Litmus28 substrate in various buffers is shown. There is only one EcoRI cleavage site in Litmus28 substrate.

FIG. 7A: EcoRI mutant K62E in NEB4 buffer.

FIG. 7B: EcoRI mutant K62A in NEB4 buffer.

FIG. 7C: WT EcoRI in EcoRI buffer (see NEB catalog 2007-8).

FIGS. 8A-B shows a comparison of EcoRI-HF and WT EcoRI in NEB4 buffer. The reaction utilized 1.2 μl lambda DNA substrate in a 2-fold serial dilution using diluent C.

FIG. 8A: WT EcoRI with a starting concentration of 19,200 U reveals a FI=4 in NEB4 buffer.

FIG. 8B: EcoRI-HF with a starting concentration of 38,400 U reveals a FI=16,000 in NEB4 buffer.

FIGS. 9A-B shows a comparison of serial dilutions of WT ScaI (4.8 U), ScaI(H193A) (9.6 U), ScaI(S201F) (19.2 U), and ScaI(H193A/S201F) (19.2 U). Each sample was initially diluted by 1/10, followed by a 2-fold serial dilution in NEB2 buffer with the specified percentage of glycerol. Each reaction mixture contains 2 μl of lambda DNA substrate (1 μg).

FIG. 9A: 5% glycerol.

FIG. 9B: 37% glycerol.

FIGS. 10A-D shows a comparison of WT ScaI and ScaI-HF (H193A/S201F). The enzymes (unit concentration as specified) were each diluted in a 2.5-fold serial dilution with diluent A. The reaction mixture contains 2 μl lambda DNA substrate and NEB1-4 buffers.

FIG. 10A: cleavage in NEB2 buffer.

FIG. 10B: cleavage in NEB4 buffer.

FIG. 10C: cleavage in NEB1 buffer.

FIG. 10D: cleavage in NEB3 buffer.

FIGS. 11A-H show the FI determination for SalI-HF and WT SalI. Both enzymes were diluted in 2-fold serial dilutions using diluent A. The reaction mixture contains 2 μl HindIII-digested lambda DNA substrate.

FIGS. 11A, B, C and D show a serial dilution of 1,200 U, 1,200 U, 300 U and 1,200 U of SalI-HF demonstrating a FI≥1,000, FI≥2,000, FI≥500 and FI≥2,000 in NEB1, 2, 3 and 4 buffers, respectively.

FIGS. 11E, F, G, and H show a serial dilution of 19.2 U, 150 U, 9,600 U and 38.4 U of WT SalI demonstrating a FI=8, FI=1, FI=32 and FI=1 in NEB1, 2, 3 and 4 buffers, respectively.

FIGS. 12A-B show the results of a 2-fold serial dilution of SphI-HF (19,200 U) in diluent A and WT SphI (143,600 U) in diluent B reacted in NEB 4 buffer with 1.2 μl lambda DNA substrate.

FIG. 12A shows cleavage by WT SphI.

FIG. 12B shows cleavage by SphI-HF.

FIGS. 13A-B show cleavage of 1.2 μl lambda DNA substrate using 2-fold serial dilutions of PstI-HF (300 U and 150 U) and 2-fold serial dilutions of WT PstI (2,400 U and 4,800 U) in NEB3 and NEB4 buffers, respectively. Serial dilutions were performed in diluent C.

FIG. 13A shows cleavage by PstI-HF in NEB4 buffer (upper panel) and NEB3 buffer (lower panel).

FIG. 13B shows cleavage by WT PstI in NEB4 buffer (upper panel) and NEB3 buffer (lower panel).

FIGS. 14A-B show cleavage of 1.2 μl lambda DNA substrate using 2-fold serial dilutions of NcoI-HF (4,800 U and 600 U) and 2-fold serial dilutions of WT NcoI (4,800 U and 1,200 U) in NEB3 and NEB4 buffers, respectively. Serial dilutions were performed in diluent A.

FIG. 14A shows cleavage by NcoI-HF in NEB4 buffer (upper panel) and NEB3 buffer (lower panel).

FIG. 14B shows cleavage by WT NcoI in NEB4 buffer (upper panel) and NEB3 buffer (lower panel).

FIGS. 15A-B show cleavage of 1.2 μl pXba DNA substrate using 2-fold serial dilutions of NheI-HF (287,200 U and 76.8 U) and 2-fold serial dilutions of WT NheI (9,600 U and 300 U) in NEB3 and NEB4 buffers, respectively. Serial dilutions were performed in diluent A.

FIG. 15A shows cleavage by NheI-HF in NEB4 buffer (upper panel) and NEB3 buffer (lower panel).

FIG. 15B shows cleavage by WT NheI in NEB4 buffer (upper panel) and NEB3 buffer (lower panel.) FIGS. 16A-B show cleavage of 1.2 μl lambda DNA substrate using 2-fold serial dilutions of SspI-HF (9,600 U and 38.4 U) and 2-fold serial dilutions of WT SspI (19,200 U and 19,200 U) in NEB3 and NEB4 buffers, respectively. Serial dilutions were performed in diluent C.

FIG. 16A shows cleavage by SspI-HF in NEB4 buffer (upper panel) and NEB3 buffer (lower panel).

FIG. 16B shows cleavage by WT SspI in NEB4 buffer (upper panel) and NEB3 buffer (lower panel).

FIGS. 17A-B show cleavage of 1.2 μl pXba DNA substrate using 2-fold serial dilutions of NotI-HF (287,200 U and 19,200 U) and 2-fold serial dilutions of WT NotI (19,200 U and 76,800 U) in NEB3 and NEB4 buffers, respectively. Serial dilutions were performed in diluent C.

FIG. 17A shows cleavage by NotI-HF in NEB4 buffer (upper panel) and NEB3 buffer (lower panel).

FIG. 17B shows cleavage by WT NotI I in NEB4 buffer (upper panel) and NEB3 buffer (lower panel).

FIGS. 18A-B show cleavage of 1.2 μl pXba DNA substrate using 2-fold serial dilutions of SacI-HF (4,800 U and 76.8 U) and 2-fold serial dilutions of WT SacI (19,200 U and 1200 U) in NEB3 and NEB4 buffers, respectively. Serial dilutions were performed in diluent A.

FIG. 18A shows cleavage by SacI-HF in NEB4 buffer (upper panel) and NEB3 buffer (lower panel).

FIG. 18B shows cleavage by WT SacI in NEB4 buffer (upper panel) and NEB3 buffer (lower panel).

FIGS. 19A-B show cleavage of 0.6 μl pBR322 DNA substrate using 2-fold serial dilutions of PvuII-HF (9,600 U and 19.2 U) and 2-fold serial dilutions of WT PvuII (19,200 U and 300 U) in NEB3 and NEB4 buffers, respectively. Serial dilutions were performed in diluent A.

FIG. 19A shows cleavage by PvuII-HF in NEB4 buffer (upper panel) and NEB3 buffer (lower panel) FIG. 19B shows cleavage by WT PvuII in NEB4 buffer (upper panel) and NEB3 buffer (lower panel).

FIGS. 20A-B show cleavage of 1.2 μl lambda DNA substrate using 2-fold serial dilutions of MfeI-HF (300 U and 19.2 U) and 2-fold serial dilutions of WT MfeI (2,400 U and 38.4 U) in NEB3 and NEB4 buffers, respectively. Serial dilutions were performed in diluent A.

FIG. 20A shows cleavage by MfeI-HF in NEB4 buffer (upper panel) and NEB3 buffer (lower panel).

FIG. 20B shows cleavage by WT MfeI in NEB4 buffer (upper panel) and NEB3 buffer (lower panel).

FIGS. 21A-B show cleavage of 1.2 μl lambda DNA substrate using a 2-fold serial dilution of HindIII-HF (4,800 U and 1,200 U) and a 2-fold serial dilution of WT HindIII (9,600 U and 4,800 U) in NEB3 and NEB4 buffers, respectively. Serial dilutions were performed in diluent A.

FIG. 21A shows cleavage by HindIII-HF in NEB4 buffer (upper panel) and NEB3 buffer (lower panel).

FIG. 21B shows cleavage by WT HindIII in NEB4 buffer (upper panel) and NEB3 buffer (lower panel) FIGS. 22A-B show cleavage of 1.2 μl lambda DNA substrate in a 2-fold serial dilution using diluent C of SbfI-HF (starting concentration: 76,800 U) and WT SbfI (starting concentration: 76,800 U) in NEB4 buffer.

FIG. 22A shows cleavage by WT SbfI.

FIG. 22B shows cleavage by SbfI-HF.

FIGS. 23A-B shows cleavage of 1.2 μl pXba DNA substrate in a 2-fold serial dilution of EagI-HF (1,200 U and 600 U) and a 2-fold serial dilution of WT EagI (150 U and 38.4 U) in NEB2 and NEB1 buffers, respectively, using diluent C.

FIG. 23A shows cleavage by EagI-HF in NEB2 buffer (upper panel) and NEB1 buffer (lower panel).

FIG. 23B shows cleavage by WT EagI in NEB2 buffer (upper panel) and NEB1 buffer (lower panel).

FIGS. 24A-B show cleavage of 1.2 μl pXba DNA substrate in a two-fold serial dilution using diluent A of EcoRV-HF (starting concentration: 38,400 U) and WT EcoRV (starting concentration: 2400 U) in NEB4 buffer.

FIG. 24A shows cleavage by WT EcoRV.

FIG. 24B shows cleavage by EcoRV-HF.

FIGS. 25A-B show cleavage of 1.2 μl T7 DNA substrate in a two-fold serial dilution using diluent A of AvrII-HF (starting concentration: 1,200 U) and WT AvrII (starting concentration: 1,200 U) in NEB4 buffer.

FIG. 25A shows cleavage by WT AvrII.

FIG. 25B shows cleavage by AvrII-HF.

FIGS. 26A-B show cleavage of 1.2 μl lambda DNA substrate by a two-fold serial dilution in diluent A of BstXI-HF (starting concentration: 300 U) and WT BstXI (starting concentration: 38.4 U) in NEB4 buffer. The reaction was performed at 55° C.

FIG. 26A shows cleavage by WT BstXI.

FIG. 26B shows cleavage by BstXI-HF.

FIGS. 27A-B show cleavage of 1.2 μl pXba DNA substrate in a two-fold serial dilution using diluent A of PciI-HF (starting concentration: 600 U) and WT PciI (starting concentration 300 U) in NEB4 buffer.

FIG. 27A shows cleavage by WT PciI.

FIG. 27B shows cleavage by PciI-HF.

FIGS. 28A-B show cleavage of 1.2 μl lambda DNA substrate in a two-fold serial dilution using diluent A of HpaI-HF (starting concentration: 4,800 U) and WT HpaI (starting concentration 9,600 U) in NEB2 buffer.

FIG. 28A shows cleavage by WT HpaI.

FIG. 28B shows cleavage by HpaI-HF.

FIGS. 29A-B show cleavage of 1.2 μl pXba DNA substrate in a two-fold serial dilution of AgeI-HF using diluent C (starting concentration: 600 U) and WT AgeI (starting concentration 600 U) in NEB4 buffer.

FIG. 29A shows cleavage by WT AgeI.

FIG. 29B shows cleavage by AgeI-HF.

FIGS. 30A-B show cleavage of 1.2 μl lambda DNA substrate in a two-fold serial dilution using diluent A of BsmBI-HF (starting concentration: 300 U) and WT BsmBI (starting concentration 4,800 U) in NEB4 buffer. The reaction is at 55° C.

FIG. 30A shows cleavage by WT BsmBI.

FIG. 30B shows cleavage by BsmBI-HF.

FIGS. 31A-B show the DNA sequence (SEQ ID NO:1) and protein sequence (SEQ ID NO:2) of MluCIM, respectively, for expression of EcoRI and MfeI.

FIG. 32 shows the DNA sequence (SEQ ID NO:3) of Hpy166IIM for expression of SalI.

FIGS. 33A-B show the DNA sequence (SEQ ID NO:4) and protein sequence (SEQ ID NO:5) of MfeI, respectively.

FIGS. 34A-B show the DNA sequence (SEQ ID NO:6) and protein sequence (SEQ ID NO:7) of BstXI, respectively.

FIGS. 35A-B show the DNA sequence (SEQ ID NO:8) and protein sequence (SEQ ID NO:9 of M.BstXI, respectively.

FIGS. 36A-B show the DNA sequence (SEQ ID NO:10) and protein sequence (SEQ ID NO:11) of S.BstXI, respectively.

FIGS. 37A-B show the DNA sequence (SEQ ID NO:12) and protein sequence (SEQ ID NO:13) of PciI, respectively.

FIGS. 38A-B show the DNA sequence (SEQ ID NO:14) and protein sequence (SEQ ID NO:15) of M.PciI, respectively.

FIG. 39 shows the DNA sequence (SEQ ID NO:17) encoding MI.EarI that recognizes CTCTTC and methylates at N4 cytosine or N6 adenine for cloning SapI and BspQI.

FIG. 40 shows the DNA sequence (SEQ ID NO:18) encoding M2.EarI that recognizes CTCTTC and methylates at N4 cytosine or N6 adenine for cloning SapI and BspQI.

FIGS. 41A-B show agarose gels to determine the FI of 1 μl WT BspQI and 1 μl mutant (K279P/R388F) BspQI (starting concentrations 512 U and 1,024 U, respectively) by cleaving 1 μl pUC19 DNA substrate in a two-fold serial dilution using diluent A and NEB1 buffer plus 10% glycerol. The reaction was conducted at 50° C.

FIG. 42 shows agarose gels to determine the FI of 5 μl WT SapI and 5 μl mutant (K273A) SapI (starting concentrations 32 U and 16 U, respectively) by cleaving pUC19 DNA substrate in a two-fold serial dilution using diluent A and NEB2 buffer plus 25% DMSO.

FIGS. 43A-B show catalytic and star activity of pXba by 5 μl WT KpnI and 5 μl D16N/E132A/D148E KpnI (initial concentrations 32 U and 256 U, respectively). The enzyme digested 2 μl pXba DNA substrate (0.5 μg) in a 2-fold serial dilution in NEB2 buffer using a diluent containing 10 mM Tris-HCl, pH 7.4, 50 mM KCl, 0.1 mM EDTA, 1 mM DTT and 50% glycerol with the total volume made up to 50 μl with water.

FIG. 43A shows the cleavage results of KpnI.

FIG. 43B shows the cleavage results of D16N/E132A/D148E KpnI.

FIG. 44A-G show the amino acid sequences of the enzymes in Table 1 and the DNA sequences of the enzymes in Table 1 that have not been previously disclosed.

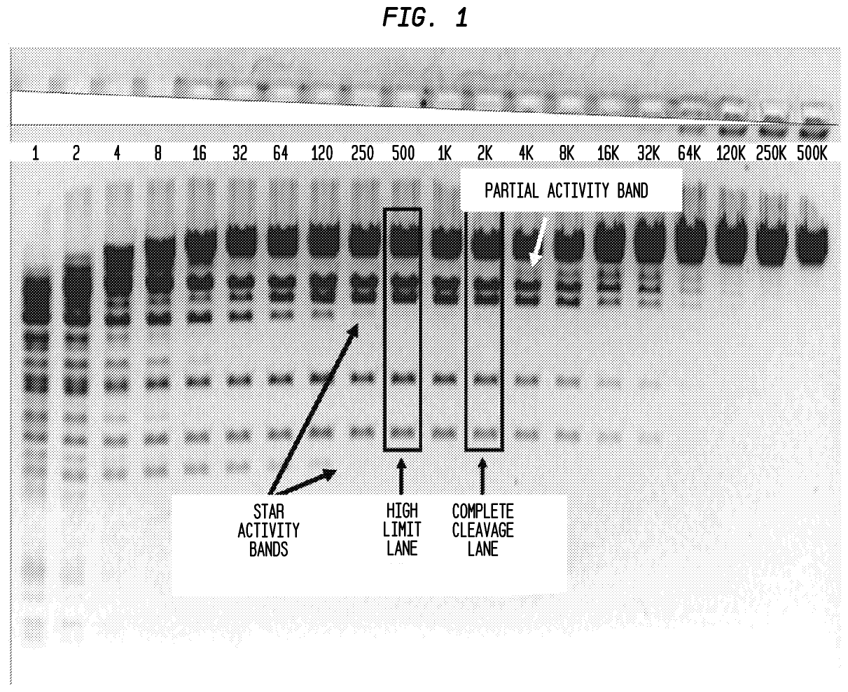

### DETAILED DESCRIPTION OF THE EMBODIMENTS

Embodiments of the invention provide a general method for selecting for restriction endonucleases with desired characteristics. The general method relies on a suitable assay for determining whether the desired restriction endonuclease has been created. In particular an embodiment of the general method provides a systematic screening method with a set of steps. This method has been deduced by performing many hundreds of reactions using many restriction endonucleases. The majority of the examples provided herein relate to identifying restriction endonucleases with reduced star activity but with cleavage activity that is at least similar to the WT restriction endonuclease. However, it is expected that the same methodology can be applied successfully to modifying other properties of the restriction endonucleases relating, for example, to improved cleavage activity in desired buffers, thermostability, rate of reaction in defined conditions, etc.

As discussed above, an end point of interest is to transform restriction endonucleases with star activity into high fidelity restriction endonucleases with significantly reduced star activity. Star activity refers to promiscuity in cleavage specificity by individual restriction endonucleases. The terms “reduction in star activity” and “increase in fidelity” are used interchangeably here. Although restriction endonucleases are characterized by their property of cleaving DNA at specific sequences, some restriction endonucleases additionally cleave DNA inefficiently at secondary sites in the DNA. This secondary cleavage may occur consistently or may arise only under certain conditions such as any of: increased concentrations, certain buffers, temperature, substrate type, storage, and incubation time.

It is generally acknowledged that little is known about the complex environment generated by the hundreds of amino acids that constitute a protein and determine specificity. One approach in the prior art has been to utilize crystallography to identify contact points between an enzyme and its substrate. Nonetheless, crystallography has limitations with respect to freezing a structure in time in an unnatural chemical environment.

The rules that determine the contribution of amino acids at any site in the protein and the role played by the structure of the substrate molecule has proved elusive using existing analytical techniques. For example, it is shown here that mutating an amino acid in a restriction endonuclease can cause all or partial loss of activity.

In this context, no structural explanation has been put forward to explain why star activity could increase with high glycerol concentration (>5% v/v), high enzyme to DNA ratio (usually >100 units of enzyme per μg of DNA), low ionic strength (<25 mM salt), high pH (>8.0), presence of organic solvent (such as DMSO, ethanol), and substitution of Mg²⁺ with other divalent cations (Mn²⁺, Co²⁺). It was here recognized that because of the diversity of factors affecting star activity, it would be necessary to conduct comparisons of WT and mutant star activity under the same reaction conditions and in the same predetermined buffer and to develop a standard reaction condition in which any high fidelity enzyme must be capable of showing the described characteristics even if these characteristics were also observed in other reaction conditions.

Present embodiments of the invention are directed to generating modified restriction endonucleases with specific improved properties, namely enhanced cleavage fidelity without significant reduction in overall cleavage activity or significant loss of yield from the host cells that make the protein. The methods that have been developed here for finding mutants with improved properties have resulted from exhaustive experimentation and the properties of the resultant enzymes have been defined in the context of specified conditions. The methods described herein may be used for altering the enzymatic properties of any restriction endonuclease under predetermined conditions, but are not limited to the specific defined conditions.

| Restriction | Steps Used to Generate a High Fidelity |
|---|---|
| Endonuclease | Restriction Endonuclease |
| BamHI | Comparison of isochizomer |
| | Targeted 22 residues to mutate to Ala. 14 mutants |
| | obtained, 3 had improved fidelity |
| | Saturation mutagenesis on 2 residues-K30 and E86 |
| | Recovered E86P as preferred mutant with greatest reduced |
| | star activity in selected buffers. Added mutations to E86P. |
| | Second round of mutation (Arg, Lys, His, Asp, Glu, Ser, Thr) |
| | to Ala and Tyr to Phe. Selected E167 and Y165 for saturation |
| | mutagenesis and selected E167T and Y165F. |
| | E163A/E167T was selected as preferred high fidelity mutant |
| | (BamHI-HF). |
| EcoRI | Comparison of isoschizomer |
| (Ex. 2) | Targeted 42 charged residues to mutate to Ala. No high |
| | fidelity mutants |
| | Second round mutations: Target additional 32 charged residues |
| | to mutate to Ala. Identified K62A. |
| | Saturation mutagenesis on K62A. EcoRI( K62E) was selected |
| | as a preferred high fidelity mutant (EcoRI-HF). |
| ScaI | Comparison of isochizomers. |
| (Ex. 3) | Targeted 58 charged residues to mutate to Ala. Identify 4 mutants |
| | Preferred mutant of 4 is (H193A/S201F). This is selected as a |
| | preferred high fidelity mutant (ScaI-HF) |
| SalI | Targeted 86 charged residues and mutate to Ala. SalI (R107A) |
| (Ex. 4) | was preferentially selected as a preferred high fidelity mutant |
| | SalI-HF). |
| SphI | Target 71 charged residues and mutate to Ala. SphI (K100A) |
| (Ex. 5) | was preferentially selected as a preferred high fidelity mutant |
| | (SphI-HF) |
| PstI | Target 92 charged amino acids and mutate to Ala. PstI (D91A) |
| (Ex. 6) | was preferentially selected as a preferred high fidelity mutant |
| | (PstI-HF) |
| NcoI | Target 66 charged residues and mutate to Ala. NcoI |
| (Ex. 7) | (A2T/R31A) was preferentially selected as a preferred high |
| | fidelity mutant (NcoI-HF). |
| NheI | Target 92 charged residues and mutate to Ala. NheI (E77A) |
| (Ex. 8) | was preferentially selected as a preferred high fidelity mutant |
| | (NheI-HF) |
| SspI | Target 81 charged residues and mutate to Ala. No preferential |
| (Ex. 9) | mutants obtained. |
| | Target 95 residues to additional charged residues and |
| | hydroxylated residues to Ala except Tyr. Tyr mutated to Phe. |
| | SspI (Y98F) was preferentially selected as a preferred high |
| | fidelity mutant (SspI-HF) |
| NotI | Target 97 charged residues and mutate to Ala. K150A was |
| (Ex. 10) | preferentially selected as a preferred high fidelity mutant |
| | (NotIHF) |
| SacI | Target 101 charged residues and mutate to Ala. SacI |
| (Ex. 11) | (Q117H/R200A) was preferentially selected as a preferred high |
| | fidelity mutant (SacI-HF) where Q117H was a carry over mutation |
| | from template with no affect on activity |
| PvuII | Target 47 charged residues and mutate to Ala. No preferred |
| (Ex. 12) | mutants obtained |
| | Target 19 hydroxylated residues-Ser/Thr and Tyr. Selected T46A |
| | for further improvement |
| | Saturation mutagensis results in a preferred mutant T46G, T46H, |
| | T64K, T46Y. PvulI(T46G) was preferentially selected as a |
| | preferred high fidelity mutant (PvulI-HF) |
| MfeI | Target 60 charged residues and mutant to Ala. No preferred |
| (Ex. 13) | mutants obtained |
| | Target 26 hydroxylated residues and mutate to Ala except for |
| | Tyr which was changed to Phe. |
| | Target 38 residues (Cys, Phe, Met, Asn, Gln, Trp) and mutate to |
| | Ala |
| | Identify Mfe (Q13A/F35Y) as a preferred high fidelity mutant |
| | (MfeI-HF) where F35Y is carried from the template |
| HindIII | Target 88 charged residues and mutate to Ala. No preferred |
| (Ex. 14) | mutants obtained. |
| | Target 103 residues (Cys Met Asn, Gln, Ser Thr Trp) and |
| | mutate to Ala and Tyr changed to Phe. |
| | Identify HindIII (K198A) as a preferred high fidelity mutant |
| | (hindIII-HF) |
| SbfI | Target 78 charged residues mutated to Ala |
| (Ex. 15) | Target 41 residues (Ser Thr) mutated to Ala/Tyr to Phe |
| | Target 55 residues of Cys, Phe, Met, Asn, Gln. Trp to Ala |
| | SbfI (K251A) was selected as a preferred high fidelity mutant |
| | (SbfI-HF) |
| EagI | Target 152 residues (Asp, Glu, His, Lys, Arg, Ser, thr, Asn, and |
| (Ex. 16) | Gln changed to Ala and Tyr changed to Phe). |
| | EagI H43A was selected as a preferred high fidelity mutant |
| | (EagIHF) |
| EcoRV | Target 162 residues (Cys, Asp, Glu, Phe, his, Lys, Met, Asn, |
| (Ex. 17) | Gln, Arg, Ser, Thr, to Ala and Trp to Phe) |
| | EcoRV (D19A/E27A) was selected as a preferred high fidelity |
| | mutant (EcoRV-HF) |
| AvrII | Target 210 residues (Cys, Asp, Glu, Phe, his, Lys, Met, Asn, Gln, |
| (Ex. 18) | Arg, Ser, Thr, to Ala and Trp to Phe) |
| | AvrII (Y104F) was selected as a preferred high fidelity mutant |
| | (AvrII-HF) |
| BstXI | Target 237 residues (Cys, Asp, Glu, Phe, his, Lys, Met, Asn, Gln, |
| (Ex. 19) | Arg, Ser, Thr, to Ala and Trp to Phe) |
| | BstXI (N65A) was selected as a preferred high fidelity mutant |
| | (BstXI-HF) |
| PciI | Target 151 residues (Cys, Asp, Glu, Phe, his, Lys, Met, Asn, Gln, |
| (Ex. 20) | Arg, Ser, Thr, to Ala and Trp to Phe) |
| | PciI (E78A/S133A) was selected as a preferred high fidelity |
| | mutant. (PciI-HF) This was spontaneous and not one of the 151 |
| | separate mutations. |
| HpaI | Target 156 residues (Cys, Asp, Glu, Phe, his, Lys, Met, Asn, Gln, |
| (Ex. 21) | Arg, Ser, Thr, to Ala and Trp to PHe) |
| | HpaI (E56A) was selected as a preferred high fidelity mutant |
| | (HpaI-HF) |
| AgeI | Target 149 residues (Cys, Asp, Glu, Phe, his, Lys, Met, Asn, Gln, |
| (Ex. 22) | Arg, Ser, Thr, to Ala and Trp to Phe) |
| | AgrI (R139A) was selected as a preferred high fidelity mutant |
| | (AgeI-HF) |
| BsmBI | Target 358 residues (Cys, Asp, Glu, Phe, his, Lys, Met, Asn, Gln, |
| (Ex. 23) | Arg, Ser, Thr, to Ala and Trp to Phe) |
| | BsmBI(N185Y/R232A) was selected as a preferred high fidelity |
| | mutant (BsmBI (HF) |
| BspQI | Target 122 residues (Arg, Lys, His, Glu, Asp, Gln, Asn, Cys) |
| (Ex. 24) | Replace R at position 279 with Phe, Pro, Tyr, Glu, Asp or Leu. |
| | Preferred mutations were R388F and K279P. |
| | Created a double mutant BspQI(K279P/R388F) as preferred high |
| | fidelity mutant (BspQI-HI) |
| SapI | Find K273 and R380 in SapI corresponding to R388 and K279 in |
| (Ex. 25) | BspQI. |
| | SapI (K273P/R380F) was selected as a preferred high fidelity |
| | mutant (SapI-HF) |
| KpnI | Target all residues (Asp, Glu, Arg, Lys, His, Thr, Tyr, Asn, |
| (Ex. 26) | Gln, Phe, Trp, Cys, Met) to Ala. |
| | More mutation was done on site D16 and D148. |
| | A combined D16N/E132A/D148E was selected as a preferred |
| | high fidelity mutant (KpnI-HF). |
| BsaI | Find 11 amino acids corresponding to the site in BsmBI. |
| (Ex. 27) | BsaI (Y231F) was selected as a preferred high fidelity mutant |
| | (BasI-HF). |

The method follows from the realization that amino acids responsible for cognate activity and star activity are different. The engineering of high fidelity restriction endonucleases described herein demonstrates that cognate activity and star activity can be separated and there are different critical amino acid residues that affect these different activities. The locations of amino acids that are here found to affect star activity are not necessarily found within the active site of the protein. The cleavage properties of any restriction endonuclease has been determined here for the first time by developing a criterion of success in the form of determining a FI (see also Wei et al. Nucleic Acid Res., 36, 9, e50 (2008)) and an overall fidelity index improvement factor.

An “overall fidelity index improvement factor” refers to the highest FI for a mutant with maximum cleavage activity divided by the highest FI of the corresponding WT endonuclease with maximum cleavage activity within a selected set of buffers. The selected set may be of any size greater than one but practically will contain less than 10 different buffers and more preferably contains 4 buffers. The set may also include less than 4 buffers.

The overall FI improvement factor of at least two should preferably be applicable for any mutant restriction endonuclease in the claimed invention additionally but not exclusively to the set of buffers consisting of NEB1, NEB2, NEB3 and NEB4.

A “similar cleavage activity” can be measured by reacting the same amount of enzyme with the same amount and type of substrate under the same conditions and visually comparing the cleavage profiles on a gel after electrophoresis such that the amount of cleavage product appears to be the same within a standard margin of error and wherein the quantitative similarity is more than 10%.

“Artificial” refers to “man-made”.

“Standard conditions” refers to an overall FI improvement factor calculated from results obtained in NEB1-4 buffers.

The general method described herein has been exemplified with 27 restriction endonucleases: AgeI, AvrII, BamHI, BsaI, BsmBI, BspQI, BstXI, EagI, EcoRI, EcoRV, HindIII, HpaI, KpnI, MfeI, NcoI, NheI, NotI, PciI, PstI, PvuII, SacI, SalI, SapI, SbfI, ScaI, SphI and SspI restriction endonucleases. However, as mentioned above, the method is expected to be effective for the engineering of any restriction endonuclease that has significant star activity.

Embodiments of the method utilize a general approach to create mutant restriction endonucleases with reduced star activity. For certain enzymes, it has proven useful to mutate charged residues that are determined to be conserved between two isoschizomers (see for example SapI in Example 25). In general, however, the method involves a first step of identifying all the charged and polar residues in a protein sequence for the endonuclease. For example, charged amino acids and polar residues include the acidic residues Glu and Asp, the basic residues His, Lys and Arg, the amide residues Asn and Gln, the aromatic residues Phe, Tyr and Trp and the nucleophilic residue Cys. Individual residues are targeted and mutated to an Ala and the products of these targeted mutations are screened for the desired properties of increased fidelity. If none of the mutants obtained provide a satisfactory result, the next step is to target mutations to all the hydroxylated amino acids, namely, Ser, Thr and Tyr, the preferred mutation being Ser and Thr to Ala and Tyr to Phe. It is also possible to target mutations to both classes of residues at one time as was done for Examples 16-23. The mutation to Ala may be substituted by mutations to Val, Leu or Ile.

After these analyses, if one or more of the preferred mutants generated in the above steps still have substandard performance under the selected tests, these mutants can be selected and mutated again to each of the additional possible 18 amino acids. This is called saturation mutagenesis. Saturation mutagenesis provided the preferred high fidelity mutants for EcoRI (Example 2), BamHI in part (Example 1) and PvuII (Example 12). Depending on the results of saturation mutagenesis, the next step would be to introduce additional mutations either targeted or random or both into the restriction endonuclease. In Example 11, SacI-HF includes a random mutation generated fortuitously during inverse PCR. In Example 20, PciI-HF resulted from a random mutation and not from targeted mutations. In Example 26, BspQI-HF contains two mutations that were found to act synergistically in enhancing fidelity.

The use of various methods of targeted mutagenesis such as inverse PCR may involve the introduction of non-target mutations at secondary sites in the protein. These secondary mutations may fortuitously provide the desired properties (see Example 20). It is desirable to examine those mutated enzymes with multiple mutations to establish whether all the mutations are required for the observed effect. In Example 11, Q1 17H in the double mutant had no effect on activity. In Example 20, the additional spontaneous mutation appears to be solely responsible for the observed improved fidelity, whereas in Example 24, the individual mutations acted synergistically.

In some cases, a mutation may provide an additional advantage other than improved fidelity (see for example BamHI in which either E163A or P173A causes the enzyme to become more thermolabile).

The high fidelity/reduced star activity properties of the mutants provided in the Examples were selected according to their function in a set of standard buffers. Other mutations may be preferable if different buffer compositions were selected. However, the same methodology for finding mutants would apply. Table 4 lists mutations which apply to each restriction endonuclease and provide an overall FI improvement factor in the standard buffer.

The engineering of the high fidelity restriction endonucleases to provide an overall FI improvement factor of at least 2 involves one or more of the following steps:

### 1. Assessment of the Star Activity of the WT Restriction Endonuclease

In an embodiment of the invention, the extent of star activity of a restriction endonuclease is tested by means of the following protocol: the endonuclease activity is determined for an appropriate substrate using a high initial concentration of a stock endonuclease and serial dilutions thereof (for example, two-fold or three-fold dilutions). The initial concentration of restriction endonuclease is not important as long as it is sufficient to permit an observation of star activity in at least one concentration such that on dilution, the star activity is no longer detected.

An appropriate substrate contains nucleotide sequences that are cleaved by cognate endonuclease activity and where star activity can be observed. This substrate may be the vector containing the gene for the restriction endonuclease or a second DNA substrate. Examples of substrates used in Table 2 are pBC4, pXba, T7, lambda, and pBR322.

The concentration of stock restriction endonuclease is initially selected so that the star activity can be readily recognized and assayed in WT and mutated restriction endonucleases. Appropriate dilution buffers such as NEB diluent A, B or C is selected for performing the serial dilutions according to guidelines in the 2007-08 NEB catalog. The serially diluted restriction endonuclease is reacted with a predetermined concentration of the appropriate substrate in a total reaction volume that is determined by the size of the reaction vessel. For example, it is convenient to perform multiple reactions in microtiter plates where a 30 μl reaction mixture is an appropriate volume for each well. Hence, the examples generally utilize 0.6 μg of substrate in 30 μl, which is equivalent to 1 μg of substrate in 50 μl. The amount of substrate in the reaction mixture is not critical, but it is preferred that it be constant between reactions. The cleavage reaction occurs at a predetermined temperature (for example 25° C., 30° C., 37° C., 50° C., 55° C. or 65° C.) for a standard time such as one hour. The cleavage products can be determined by any standard technique, for example, by 0.800 agarose gel electrophoresis to determine the fidelity indices as defined above.

Not all restriction endonucleases have significant star activity as determined from their FI. However, if an endonuclease has a highest FI of no more than about 250 and a lowest FI of less than 100, the restriction endonuclease is classified as having significant star activity. Such endonucleases are selected as a target of enzyme engineering to increase fidelity for a single substrate. In some cases, the restriction endonucleases with both FI over about 500 and FI less than about 100 are also engineered for better cleavage activity.

Table 2 below lists the FI of some engineered restriction endonucleases before engineering. All samples were analyzed on 0.8% agarose gel.

TABLE 2

| | Diluent | | Temp | | | | | | | | |
|---|---|---|---|---|---|---|---|---|---|---|---|
| Enzyme | (NEB) *** | Substrate * | ° C. | FI-1 ** | | FI-2 ** | | FI-3 ** | | FI-4 ** | |
| AgeI | C | pXba | 37 | 16 | (1) | 8 | (½) | 64 | (⅛) | 8 | (½) |
| AvrII | B | T7 | 37 | 64 | (1) | 8 | (1) | 32 | (¼) | 32 | (1) |
| BamHI | A | λ | 37 | 4 | (½) | 4 | (1) | 32 | (1) | 4 | (½) |
| BsaI | B | pBC4 | 50 | 8 | (¼) | 120 | (1) | 16 | (¼) | 32 | (1) |
| BsmBI | B | λ | 55 | 1 | (⅛) | 8 | (½) | 120 | (1) | 4 | (¼) |
| BspQI | B | λ | 50 | 2 | (⅛) | 16 | (1) | 32 | (1) | 4 | (½) |
| BstXI | B | λ | 55 | 2 | (½) | 2 | (½) | 2 | (⅛) | 4 | (1) |
| EagI | B | pXba | 37 | 4 | (¼) | 8 | (½) | 250 | (1) | 16 | (1) |
| EcoRI | C | λ | 37 | 250 | (½) | 4 | (1) | 250 | (1) | 4 | (1) |
| EcoRV | A | pXba | 37 | 32 | ( 1/16) | 120 | (½) | 1000 | (1) | 64 | (¼) |
| HindIII | B | λ | 37 | 32 | (¼) | 250 | (1) | 4000 | (¼) | 32 | (½) |
| HpaI | A | λ | 37 | 32 | ( 1/16) | 1 | (¼) | 2 | (⅛) | 16 | (1) |
| KpnI | A | pXba | 37 | 16 | (1) | 16 | (¼) | 8 | (1/16) | 4 | (½) |
| MfeI | A | λ | 37 | 32 | (1) | 16 | (⅛) | 8 | (1/16) | 32 | (1) |
| NcoI | A | λ | 37 | 120 | (1) | 32 | (1) | 120 | (¼) | 32 | (1) |
| NheI | C | pXba | 37 | 32 | (1) | 120 | (¼) | 120 | (⅛) | 32 | (1) |
| NotI | C | pXba | 37 | ≥32000 | ( 1/16) | 64 | (1) | 500 | (1) | 32 | (¼) |
| PciI | A | pXba | 37 | 2000 | (½) | 16 | (¼) | 120 | (1) | 8 | (⅛) |
| PstI | C | λ | 37 | 64 | (1) | 32 | (1) | 120 | (1) | 8 | (½) |
| PvuII | A | pBR322 | 37 | 250 | (1) | 16 | (¼) | 8 | (1/32) | ¼ | (1) |
| SacI | A | pXba | 37 | 120 | (1) | 120 | (½) | 120 | (1/32) | 32 | (½) |
| SalI | A | λ (H3) | 37 | 8 | (1/500) | 1 | (1/16) | 32 | (1) | 1 | (1/120) |
| SapI | C | λ | 37 | 16 | (¼) | 64 | (½) | 32 | (¼) | 16 | (1) |
| SbfI | A | λ | 37 | 32 | (1) | 8 | (¼) | 8 | (1/16) | 8 | (½) |
| ScaI | A | λ | 37 | 1/16 | ( 1/32) | ⅛ | (1) | 4 | (½) | 1/64 | ( 1/16) |
| SphI | B | λ | 37 | 64 | (1) | 32 | (1) | 64 | (¼) | 16 | (½) |
| SspI | C | λ | 37 | 64 | (1) | 16 | (1) | 32 | (¼) | 16 | (1) |

* Substrate: λ is lambda phage DNA; λ (H3) is HindIII-digested lambda phage DNA; pXba is pUC19 with XbaI-digested fragment of Adeno Virus; pBC4: a shorter version of pXba; T7: T7 DNA
** FI-1 to FI-4: fidelity index of the enzyme in NEBuffer 1, 2, 3 and 4. The number in parenthesis is a value for relative cleavage activity of the mutant restriction endonuclease in a specified buffer in a set of buffers compared with the “best” cleavage activity of the same mutant restriction endonuclease in any of the buffers in the set of buffers.
The compositions of NEB buffers follow:
NEB1: 10 mM Bis Tris Propane-HCl, 10 mM MgCl₂, 1 mM dithiothreitol (pH 7.0 at 25° C.);
NEB2: 50 mM NaCl, 10 mM Tris-HCl, 10 mM MgCl₂, 1 mM dithiothreitol (pH 7.9 at 25° C.);
NEB3: 100 mM NaCl, 50 mM Tris-HCl, 10 mM MgCl₂, 1 mM dithiothreitol (pH 7.9 at 25° C.);
NEB4: 50 mM potassium acetate, 20 mM Tris-acetate, 10 mM magnesium acetate, 1 mM dithiothreitol (pH 7.9 at 25° C.).
*** The compositions of NEB diluents follow. (Using diluents in the dilution instead of water will keep the glycerol concentration in the reaction as a constant.)
Diluent A: 50 mM KCl, 10 mM Tris-HCl, 0.1 mM EDTA, 1 mM dithiothreitol, 200 mg/ml BSA. 50% glycerol (pH 7.4 at 25° C.);
Diluent B: 300 mM NaCl, 10 mM Tris-HCl, 0.1 mM EDTA, 1 mM dithiothreitol, 500 mg/ml BSA, 50% glycerol (pH 7.4 at 25° C.);
Diluent C: 250 mM NaCl, 10 mM Tris-HCl, 0.1 mM EDTA, 1 mM dithiothreitol, 0.15% Triton X-100, 200 mg/ml BSA, 50% glycerol (pH 7.4 at 25° C.).

### 2. Construction of High Expression Host Cell Strains

It is convenient if a host cell is capable of over-expressing the mutant restriction endonuclease for which reduced star activity is sought. If the restriction enzyme is highly expressed in E. coli, the star activity can be readily detected in the crude extract, which simplifies the screening for the high fidelity restriction endonuclease. However, the mutated restriction endonuclease can be expressed in any host cell providing that the host cell is protected in some way from toxicity arising from enzyme cleavage. This might include: the presence of a methylase; production in a compartment of the cell which provides a barrier to access to the genome (such as an inclusion body or the periplasm); in vitro synthesis; production in an emulsion (see U.S. patent application Ser. No. 12/035,872) absence of cleavage sites in the host genome; manufacture of the enzyme in component parts subject to intein mediated ligation (see U.S. Pat. No. 6,849,428), etc.

Over-expression of the mutated restriction endonucleases for purposes of production can be achieved using standard techniques of cloning, for example, use of an E. coli host, insertion of the endonuclease into a pUC19-derived expression vector, which is a high copy, and use of a relatively small plasmid that is capable of constant expression of recombinant protein. The vector may preferably contain a suitable promoter such as the lac promoter and a multicopy insertion site placed adjacent to the promoter. Alternatively, a promoter can be selected that requires IPTG induction of gene expression. If the activity in the crude extract is not sufficient, a column purification step for the restriction endonuclease in crude extract may be performed.

### 3. Mutagenesis of Restriction Endonuclease

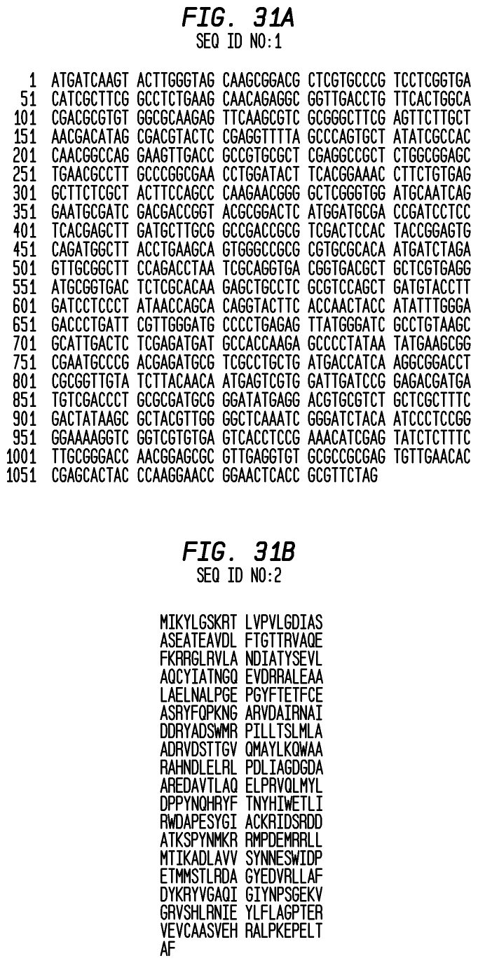

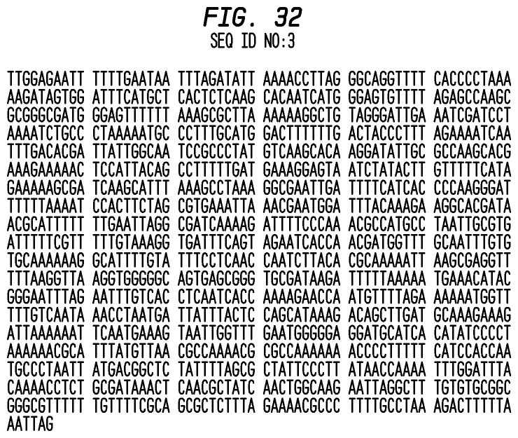

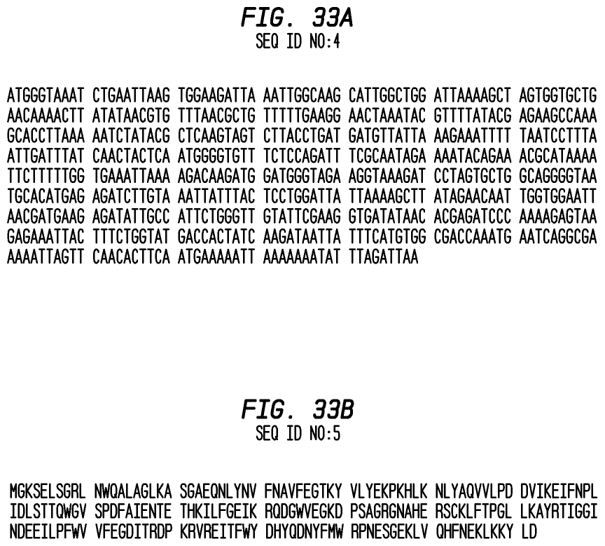

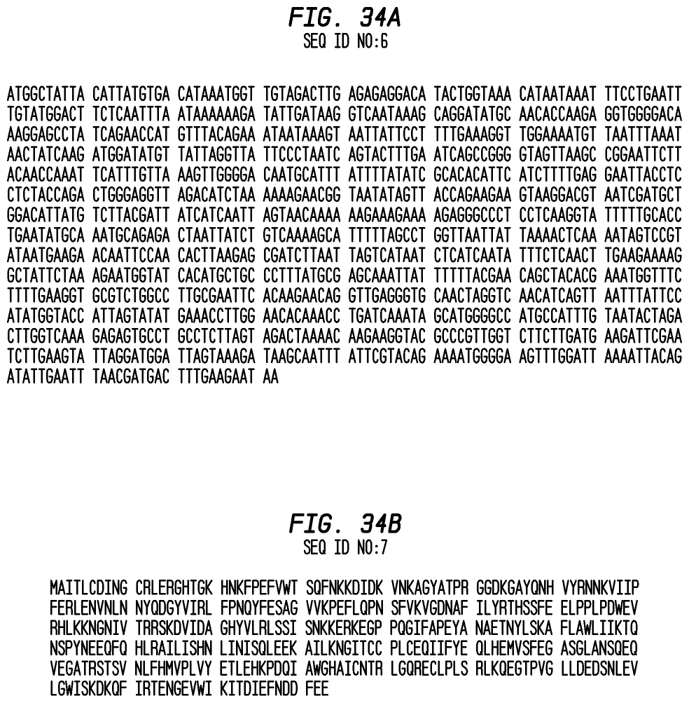

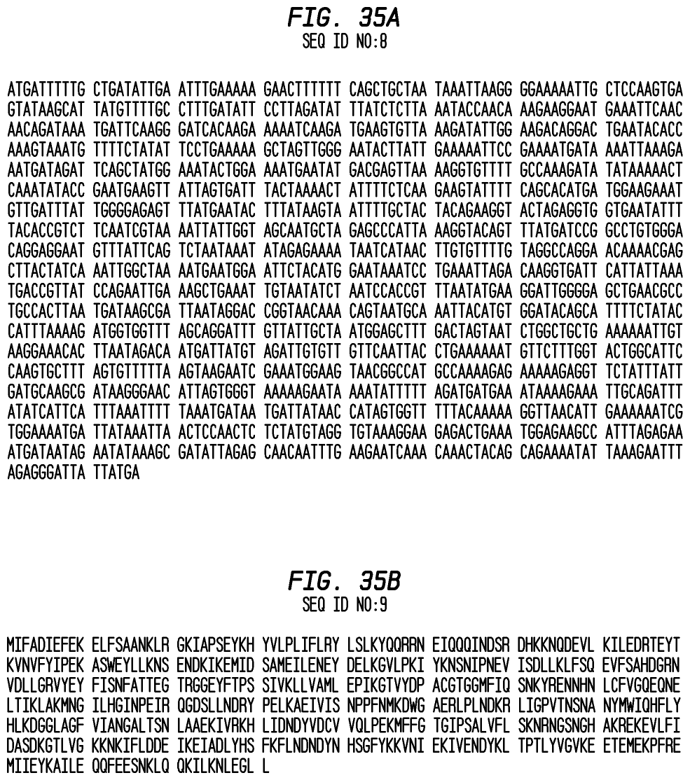

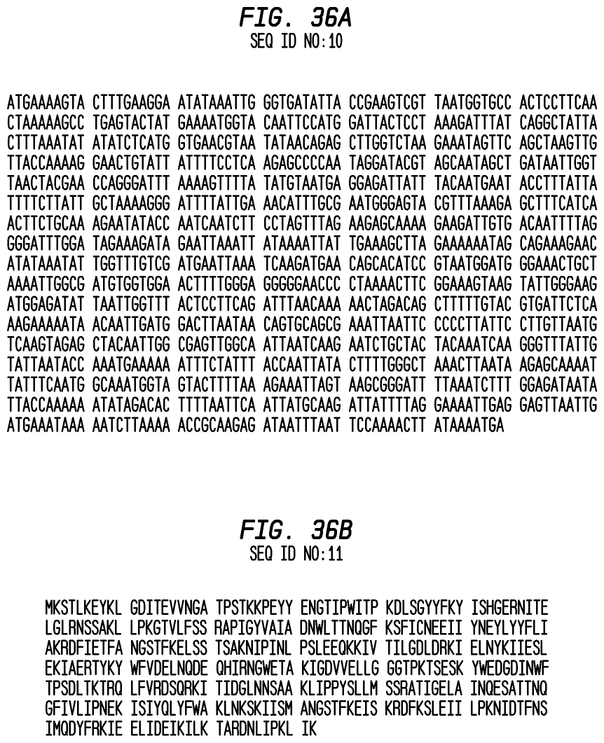

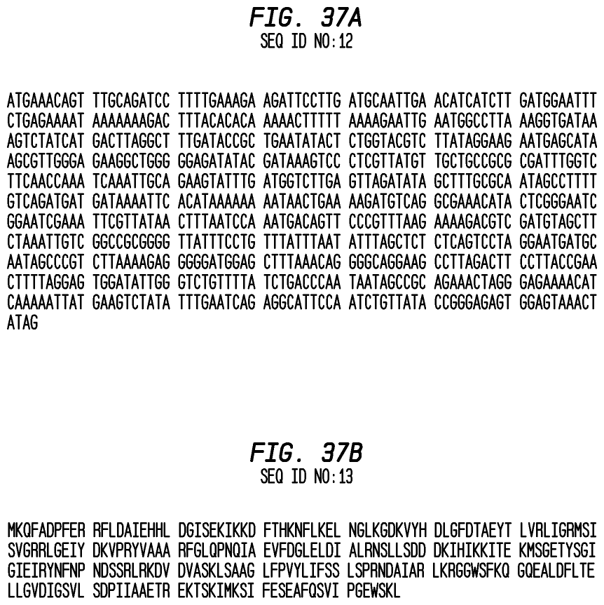

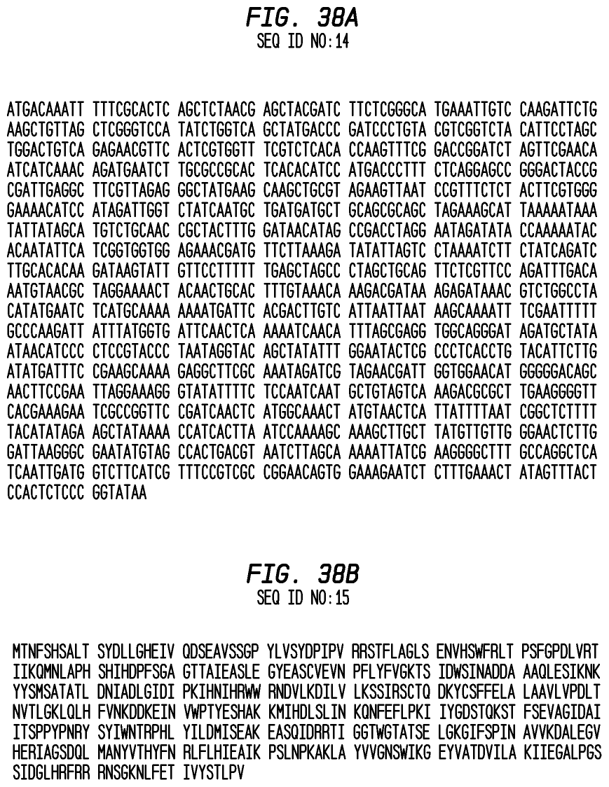

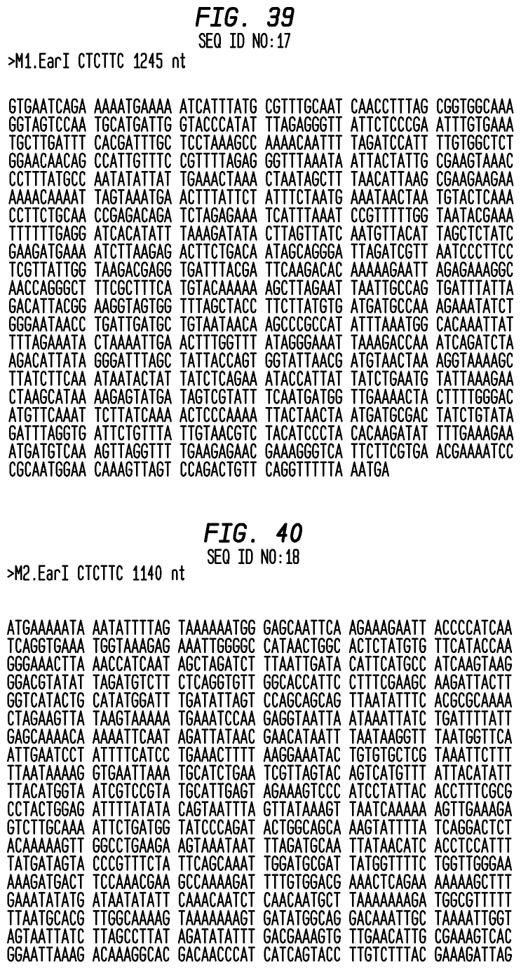

DNA encoding each charged or polar group in the restriction endonuclease may be individually targeted and the mutated DNA cloned and prepared for testing. Multiple mutations may be introduced into individual restriction endonuclease genes. Targeted mutagenesis of restriction endonucleases may be achieved by any method known in the art. A convenient method used here is inverse PCR. In this approach, a pair of complementary primers that contains the targeted codon plus a plurality of nucleotides (for Example 18 nt) on both the 5′ and 3′ side of the codon is synthesized. The selection of suitable primers can be readily achieved by reviewing the gene sequence of the endonuclease of interest around the amino acid residue of interest. Access to gene sequences is provided through REBASE and GenBank. The sequences for the endonucleases described herein in the Examples are provided in FIGS. 31 to 38 and 44. The template for PCR is a plasmid containing the restriction endonuclease gene. The polymerase is preferably a high fidelity polymerase such as Vent® or Deep Vent™ DNA polymerase. By varying the annealing temperature and Mg²⁺ concentration, successful introduction of most mutations can be achieved. The PCR amplification product is then purified and preferably digested by DpnI. In an embodiment of the invention, the digested product was transformed into competent host cells (for example, E. coli), which have been pre-modified with a corresponding methylase. Colonies from each mutant were picked and grown under similar conditions to those in which the WT is grown (for example, using similar growth medium, drug selection, and temperature). The resulting restriction endonucleases were screened for reduced star activity.

4. Screening for Mutant Restriction Endonucleases with Reduced Star Activity

Conditions such as buffer composition, temperature and diluent should be defined for determining star activity in a mutant restriction endonuclease. Tables 2 and 3 show the FI of recombinant endonucleases before and after mutation in four different buffers using three different diluents at 37° C. Accordingly, it is possible to determine which mutants have an overall desirable improved fidelity index factor of at least 2, more than 10, at least 50 or more than 500 and to select enzymes as preferred high fidelity mutants.

In an embodiment of the invention, the mutant restriction endonucleases were screened for activity in normal buffer conditions (no more than 5% glycerol) first. For those mutants with at least about 10% of activity of WT restriction endonuclease, activity was also determined in star activity promotion conditions that promoted star activity, for example, high glycerol concentration and optionally high pH. Preferably, the mutant with the least star activity but with acceptable cognate activity in normal buffers is selected. Plasmid can then be extracted and sequenced for the confirmation of the mutant. In some cases, the star activity is not easily measured, even with high glycerol and high pH conditions. Instead, the activity in different buffers is measured and compared, and the one with the highest cleavage activity ratio in NEB4 compared with NEB3 can be tested further for star activity improvement.

### 5. Saturation Mutagenesis on One Single Residue

As described in the previous section, the first step is to mutate a target amino acid in the restriction endonuclease to Ala. If the results are not satisfactory, saturation mutagenesis is performed. This is preferably performed by one of two methods. One method is to change the intended codon into NNN. After mutagenesis, multiple colonies are assayed under normal conditions and under conditions that promote star activity. Alternatively, a different codon can be selected for mutagenesis of each of the targeted amino acids for example: Ala: GCT; Cys: TGC; Asp: GAC; Glu: GAA; His: CAC; Ile: ATC; Lys: AAA; Leu: CTG; Met: ATG; Asn: AAC; Pro: CCG; Gln: CAG; Arg: CGT; Ser: TCC; Thr: ACC; Val: GTT; Trp: TGG and Tyr: TAC

### 6. Combination

More than one mutation can be introduced into the restriction endonuclease gene if a single mutation does not sufficiently reduce the star activity. Mutation combination and saturation mutagenesis can be performed in any order.

### 7. Mutant Purification and Assessment of the Improvement

The high fidelity mutants may be purified in a variety of ways including use of different chromatography columns. For normal quality assessment, one FPLC heparin column is enough to eliminate the DNA and non-specific nucleases from the preparation. Multiple columns including ion exchange, hydrophobic, size exclusion and affinity columns can be used for further purification.

Purified high fidelity restriction endonucleases are measured for FI in four NEB buffers and compared with the FIs of the WT restriction endonuclease. The ratio of FI for the high fidelity restriction endonuclease in its optimal buffer to that of WT is the overall improvement factor.

TABLE 3
FI* for exemplified restriction endonucleases

| | Diluent | | Temp | | | | | | | | |
|---|---|---|---|---|---|---|---|---|---|---|---|
| Enzyme | (NEB) | Substrate * | ° C. | FI-1 ** | | FI-2 ** | | FI-3 ** | | FI-4 ** | |
| AgeI-HF | C | pXba | 37 | ≥500 | (1) | ≥250 | (½) | ≥16 | ( 1/16) | ≥250 | (1) |
| AvrII-HF | B | T7 | 37 | 500 | (1) | ≥500 | (½) | ≥16 | ( 1/64) | ≥1000 | (1) |
| BamHI-HF | A | λ | 37 | ≥4000 | (1) | ≥4000 | (1) | ≥250 | ( 1/16) | ≥4000 | (1) |
| BsaI | B | pBC4 | 50 | ≥4000 | (½) | ≥8000 | (1) | 120 | (1) | ≥8000 | (1) |
| BsmBI | B | λ | 55 | 2 | (1) | ≥500 | (1) | ≥64 | (⅛) | ≥500 | (1) |
| BspQI-HF | A | pUC19 | 50 | ≥1000 | (¼) | ≥1000 | (¼) | ≥64 | ( 1/64) | ≥4000 | (1) |
| BstXI-HF | A | λ | 55 | ≥120 | (½) | ≥250 | (1) | ≥16 | ( 1/16) | ≥250 | (1) |
| EagI-HF | C | pXba | 37 | 250 | (½) | 250 | (1) | 250 | (½) | 500 | (1) |
| EcoRI-HF | C | λ | 37 | 2000 | (⅛) | 4000 | (¼) | 250 | ( 1/250) | 16000 | (1) |
| EcoRV-HF | A | pXba | 37 | ≥16000 | (¼) | ≥64000 | (1) | ≥32000 | (½) | ≥64000 | (1) |
| HindIII-HF | B | λ | 37 | ≥16000 | (¼) | ≥64000 | (1) | ≥16000 | (¼) | ≥32000 | (½) |
| HpaI-HF | A | λ | 37 | ≥32 | ( 1/32) | ≥2000 | (1) | 2 | (⅛) | ≥2000 | (½) |
| KpnI-HF | A | pXba | 37 | ≥4000 | (1) | ≥1000 | (¼) | ≥64 | ( 1/64) | ≥4000 | (1) |
| MfeI-HF | A | λ | 37 | ≥1000 | (1) | ≥250 | (¼) | ≥16 | ( 1/64) | ≥500 | (½) |
| NcoI-HF | A | λ | 37 | ≥4000 | (¼) | ≥4000 | (¼) | ≥1000 | ( 1/16) | ≥64000 | (1) |
| NheI-HF | C | pXba | 37 | ≥128000 | (1) | ≥4000 | ( 1/32) | ≥32 | ( 1/2000) | ≥32000 | (½) |
| NotI-HF | C | pXba | 37 | ≥8000 | ( 1/16) | ≥128000 | (1) | ≥4000 | ( 1/64) | ≥64000 | (½) |
| PciI-HF | A | pXba | 37 | NC | | ≥2000 | (1) | ≥2000 | (1) | ≥1000 | (1) |
| PstI-HF | C | λ | 37 | 1000 | (⅛) | 4000 | (½) | 4000 | (¼) | 4000 | (1) |
| PvuII-HF | A | pBR322 | 37 | ≥250 | ( 1/120) | ≥2000 | ( 1/16) | ≥250 | ( 1/120) | 500 | (1) |
| SacI-HF | A | pXba | 37 | ≥32000 | (1) | ≥16000 | (½) | ≥500 | ( 1/64) | ≥32000 | (1) |
| SalI-HF | A | λ (H3) | 37 | ≥8000 | (⅛) | ≥64000 | (1) | ≥4000 | ( 1/16) | ≥32000 | (½) |
| SbfI-HF | C | λ | 37 | 1000 | (1) | 120 | (½) | 8 | ( 1/32) | 250 | (1) |
| ScaI-HF | A | λ | 37 | 4000 | (⅛) | 1000 | (1) | 2000 | ( 1/32) | 1000 | (1) |
| SphI-HF | B | λ | 37 | 4000 | (⅛) | 2000 | ( 1/16) | 250 | ( 1/250) | 8000 | (1) |
| SspI-HF | C | λ | 37 | ≥4000 | (½) | 120 | (½) | ≥32 | ( 1/128) | 500 | (1) |

* The FI is a ratio of the highest concentration that does not show star activity to the lowest concentration that completes digestion of the substrate.
** The number in parenthesis is a value for relative cleavage activity of the mutant restriction endonuclease in a specified buffer in a set of buffers compared with the greatest cleavage activity of the same mutant restriction endonuclease in any of the buffers in the set of buffers.

TABLE 4
Mutations providing restriction endonucleases with high fidelity

| Restriction | Examples of mutants with overall |
|---|---|
| Endonuclease | improved FI factor ≥2 |
| AgeI | R139A; S201A* |
| AvrII | Y104F; M29A; E96A; K106A; S127A; F142A |
| BamHI | E163A/E167T; K30A; E86A; E86P; K87A; K87E; K87V; K87N; P144A; Y165F; |
| | E167A; E167R; E167K; E167L; E167I; K30A/E86A; E86A/K106A; |
| | K30A/E86A/K106A; K30A/K87A; E86P/K87E; E86A/Y165F; K30A/E167A; |
| | E163S/E170T/P173A; E1663S/E170T/P173A; E86P/K87T/K88N/E163S/E170T/P173A; |
| | E86P/K87R/K88G/E163S/E170T/P173A; E86P/K87P/K88R/ |
| | E163S/E170T/P173A/E211K; E86P/K87T/K88R/E163S/E170T/P173A/N158S; |
| | E86P/K87S/K88P/E163S/E170T/P173A; E86P/K87G/K88S/E163S/E170T/P173A; |
| | E86P/K87R/K88Q/E163S/E170T/P173A; E86P/K87W/K88V; E86P/P173A |
| BsaI | Y231F |
| BsmBI | N185Y/R232A; H230A; D231A; R232A; |
| BspQI | K279P/R388F; K279A; K279F; K279P; K279Y; K279E; K279D |
| | R388A; R388F; R388Y; R388L; K279P/R388F; K279A/R388A; |
| | D244A |
| BstXI | N65A; Y57F; E75A; N76A; K199A; |
| EagI | H43A |
| EcoRI | K62A; K62S; K62L; R9A; K15A; R123A; K130A; R131A; R183A; S2Y; D135A; |
| | R187A; K62E |
| EcoRV | D19A; E27A; D19A/E27A |
| HindIII | S188P/E190A; K198A |
| HpaI | Y29F; E56A |
| KpnI | D148E; D16N/R119A/D148E; D2A/D16N/D148E; D16N/E134A/D148E; |
| | D16N/E132A/D148E |
| MfeI | Y173F; Q13A/F35Y |
| NcoI | D56A; H143A; E166A; R212A; D268A; A2T/R31A |
| NheI | E77A |
| NotI | K176A; R177A; R253A; K150A |
| PciI | E78A/S133A |
| PstI | E204G; K228A; K228A/A289V; D91A |
| PvuII | T46A; T46H; T46K; T46Y; T46G |
| SacI | Q117H/R154A/L284P; Q117H/R200A |
| SalI | R82A; K93A; K101A; R107A |
| SapI | K273P; R380A; K273P/R380A |
| SbfI | K251A |
| ScaI | R18A; R112A; E119A; H193A; S201F; H193A/S201F |
| SphI | D91A; D139A; D164A; K100A |
| SspI | H65A; K74A; E78A; E85A; E89A; K109A; E118A; R177A; K197A; Y98F |

The mutations for each enzyme are separated by a semicolon.

All references cited above and below, as well as U.S. provisional application Ser. No. 60/959,203, are incorporated by reference.

### EXAMPLES

Where amino acids are referred to by a single letter code, this is intended to be standard nomenclature. The key to the code is provided for example in the NEB catalog 2007/2008 on page 280.

Plasmids used for cloning and as substrates have sequences as follows:
pLaczz2 (SEQ ID NO:102), pSyx20-lacIq (SEQ ID NO:105), pBC4 (SEQ ID NO:103), pXba (SEQ ID. NO:104) and pAGR3 (SEQ ID NO:106). pACYC is described in GenBank XO 6403, T7 in GenBank NC001604, pUC18 in GenBank L09136, and pRRS in Skoglund et al. Gene, 88:1-5 (1990. pSX33 was constructed by inserting lacI gene into pLG339 at EcoRI site. pLG339 is described in Stoker, et al. Gene 19, 335-341 (1982).

All buffers identified as NEB buffers used herein are obtainable from New England Biolabs, Inc. (NEB), Ipswich, MA.

### Example 1: Engineering of High Fidelity BamHI

### 1. Extraction of Plasmids Containing BamHI Methylase and BamHI Endonuclease

Competent E. coli host cells were transformed with pUC18-BamHIR and pACYC184-BamHIM and BamHIR was extracted by a standard Qiagen Mini-prep method using standard miniprep techniques (Qiagen, Valencia, CA).

### 2. Selection of Mutagenesis Target

BamHI and related restriction endonuclease OkrAI were cloned and sequenced. OkrAI was found to have significant star activity if the reaction occurred at 37° C. in NEB buffers (1, 2 and 4). The present analysis tested the assumption that the amino acid residue(s) responsible for the star activity were similar between BamHI and OkrAI endonuclease.

| The complete protein sequence of BamHI (SEQ ID NO: 19) is: | |
|---|---|
| 1 | MEVEKEFITD EAKELLSKDK LIQQAYNEVK TSICSPIWPA TSKTFTINNT |
| 51 | EKNCNGVVPI KELCYTLLED TYNWYREKPL DILKLEKKKG GPIDVYKEFI |
| 101 | ENSELKRVGM EFETGNISSA HRSMNKLLLG LKHGEIDLAI ILMPIKQLAY |
| 151 | YLTDRVTNFE ELEPYFELTE GQPFIFIGFN AEAYNSNVPL IPKGSDGMSK |
| 201 | RSIKKWKDKV ENK |
| The complete protein sequence of OkrAI (SEQ ID NO: 20) is: | |
| 1 | MKIKRIEVLI NNGSVPGIPM ILNEIQDAIK TVSWPEGNNS FVINPVRKGN |
| 51 | GVKPIKNSCM RHLHQKGWAL EHPVRIKAEM RPGPLDAVKM IGGKAFALEW |
| 101 | ETGNISSSHR AINKMVMGML ERVIIGGVLI LPSRDMYNYL TDRVGNFREL |
| 151 | EPYFSVWRQF NLKDAYLAIV EIEHDSVDAQ VSLIPKGTDG RAIR |

A “Bestfit” similarity analysis done by GCG for the protein sequence of BamHI and OkrAI endonuclease showed the following result where the upper protein sequence is BamHI and the bottom protein sequence is OkrAI:

The similar charged residues (D, E, H, K, R) in BamHI were found to be E28, K30, K52, K61, E77, K84, E86, K88, D94, K97, K106, E111, E113, H121, R122, K126, K146, D154, R155, E161, E163, E170, E182, K193, D196 and R201. These residues are underlined in the above comparison. Known mutants E77K, D94N, E111K and E113K were previously reported to be inactive (Xu, Shuang-yong et al. J. Bacteriol. 266: 4425-4429 (1991)) so they were excluded. The initial mutagenesis selection targeted 22 shared charged amino acid residue for mutation to Alanine: E28A, K30A, K52A, K61A, K84A, E86A, K88A, K97A, K106A, H121A, R122A, K126A, K146A, D154A, R155A, E161A, E163A, E170A, E182A, K193A, D196A and R201A.

### 3. Mutagenesis of BamHI

The point mutagenesis of the selected mutations was done by inverse PCR. The corresponding codons were all changed to GCA (alanine). The following primers were used for mutagenesis:

E28A
(SEQ ID NO: 23)
5′ATTCAACAAGCATACAATGCAGTTAAAACATCTATTGT3′
(SEQ ID NO: 24)
5′ACAAATAGATGTTTTAACTGCATTGTATGCTTGTTGAAT3′
K30A
(SEQ ID NO: 25)
5′CAAGCATACAATGAAGTTGCAACATCTATTTGTTCACCT3′
(SEQ ID NO: 26)
5′AGGTGAACAAATAGATGTTGCAACTTCATTGTATGCTTG3′
K52A
(SEQ ID NO: 27)
5′ACGATTAACAACACCGAAGCAAATTGTAACGGTGTAGTA3′
(SEQ ID NO: 28)
5′TACTACACCGTTACAATTTGCTTCGGTGTTGTTAATCGT3′
K61A
(SEQ ID NO: 29)
5′AACGGTGTAGTACCAATTGCAGAACTATGTTACACCTTA3′
(SEQ ID NO: 30)
5′TAAGGTGTAACATAGTTCTGCAATTGGTACTACACCGTT3′
K84A
(SEQ ID NO: 31)
5′AACCCCCTTGATATACTTGCACTTGAAAAGAAAAAAGGT3′
(SEQ ID NO: 32)
5′ACCTTTTTTCTTTTCAAGTGCAAGTATATCAAGGGGTTT3′
E86A
(SEQ ID NO: 33)
5′GATATACTTAAACTTGCAAAGAAAAAAGGTGGTCCG3′
(SEQ ID NO: 34)
5′CGGACCACCTTTTTTCTTTGCAAGTTTAAGTATATCAAG3′
K88A
(SEQ ID NO: 35)
5′ATACTTAAACTTGAAAAGGCAAAAGGTGGTCCGATTGAT3′
(SEQ ID NO: 36)
5′ATCAATCGGACCACCTTTTGCCTTTTTCAAGTTTAAGTAT3′
K97A
(SEQ ID NO: 37)
5′GGTCCGATTGATGTTTATGCAGAGTTCATAGAAAACAGT3′
(SEQ ID NO: 38)
5′ACTGTTTTCTATGAACTCTGCATAAACATCAATCGGACC3′
K106A
(SEQ ID NO: 39)
5′ATAGAAAAACAGTGAACTTGCACGTGTAGGTATGGAA3′
(SEQ ID NO: 40)
5′AAATTCCATACCTACACGTGCAAGTTCACTGTTTTCTAT3′
H121A
(SEQ ID NO: 41)
5′GGAAATATTAGTTCTGCCGCACGTTCAATGAACAAACTT3′
(SEQ ID NO: 42)
5′AAGTTTGTTCATTGAAACGTGCGGCAGAACTAATATTCC3′
R122A
(SEQ ID NO: 43)
5′AATATTAGTTCTGCCCACGCATCAATGAACAAACTTCTA3′
(SEQ ID NO: 44)
5′TAGAAGTTTGTTCATTGATGCGTGGGCAGAACTAATATT3′
K126A
(SEQ ID NO: 45)
5′GCCCACCGTTCAATGAACGCACTTCTATTAGGATTAAAACAT3′
(SEQ ID NO: 46)
5′ATGTTTTAATCCTAATAGAAGTGCGGTCATTGAACGGTGGGC3′
K146A
(SEQ ID NO: 47)
5′ATTATCCTTATGCCTATTGCACAATTGGCCTATTATCTT3′
(SEQ ID NO: 48)
5′AAGATAATAGGCCAATTGTGCAATAGGCATAAGGATAAT3′
D154A
(SEQ ID NO: 49)
5′TTGGCCTATTATCTTACAGCACGTGTTACCAATTTCGAG3′
(SEQ ID NO: 50)
5′CTCGAAATTGGTAACACGTGCTGTAAGATAATAGGCCAA3′
R155A
(SEQ ID NO: 51)
5′GCCTATTATCTTACAGATGCAGTTACCAATTTCGAGGAA3′
(SEQ ID NO: 52)
5′TTCCTCGAAATTGGTAACTGCATCTGTAAGATAATAGGC3′
E161A
(SEQ ID NO: 53)
5′CGTGTTACCAATTTCGAGGCATTAGAACCTTATTTTGAA3′
(SEQ ID NO: 54)
5′TTCAAAATAAGGTTCTAATGCCTCGAAATTGGTAACACG3′
E163A
(SEQ ID NO: 55)
5′ACCAATTTCGAGGAATTAGCACCTTATTTTGAACTTACT3′
(SEQ ID NO: 56)
5′AGTAAGTTCAAAATAAGGTGCTAATTCCTCGAAATTGGT3′
E170A
(SEQ ID NO: 57)
5′CCTTATTTTGAACTTACTGCAGGACAACCATTTATTTTTATT3′
(SEQ ID NO: 58)
5′AATAAAAATAAATGGTTGTGCTGCAGTAAGTTCAAAATAAGG3′
E182A
(SEQ ID NO: 59)
5′TTTATTTTTATTGGATTTAATGCTGCAGCTTATAATTCTAATGTC3′
(SEQ ID NO: 60)
5′GACATTAGAATTATAAGCTGCAGCATTAAATCCAATAAAAATAAA3′
K193A
(SEQ ID NO: 61)
5′AATGTCCCTTTAATTCCCGCAGGTTCTGACGGTATGTCA3′
(SEQ ID NO: 62)
5′TGACATACCGTCAGAACCTGCGGGAATTAAAGGGACATT3′
D196A
(SEQ ID NO: 63)
5′TTAATTCCCAAAGGTTCTGCAGGTATGTCAAAACGCTCA3′
(SEQ ID NO: 64)
5′TGAGCGTTTTGACATACCTGCAGAACCTTTGGGAATTAA3′
R201A
(SEQ ID NO: 65)
5′TCTGACGGTATGTCAAAAGCATCAATTAAGAAATGGAAA3′
(SEQ ID NO: 66)
5′TTTCCATTTCTTAATTGATGCTTTTGACATACCGTCAGA3′

The PCR reaction in a reaction volume of 100 μl, contained 2 μl of each PCR primer, 1 μl pUC18-bamhiR, 400 μM dNTP, 4 units of Deep Vent™ DNA polymerase, and 10 μl 10× Thermopol buffer containing 0, 2, or 6 μl MgSO₄ with additional water.

The PCR reaction conditions were 94° C. for 5 min, followed by 25 cycles of 94° C. 30 sec, 55° C. 30 sec, 72° C. 4 min and a final extension time at 72° C. for 7 mins. The PCR product was purified on a standard Qiagen spin column (Qiagen, Valencia, CA). Six to sixteen μl of PCR product was digested by 20 units of DpnI for 1 hour. The digested product was 30 transformed into E. coli (pACYC-bamHIM).

After six PCR reactions, 14 out of the engineered 22 mutations were obtained: E28A, K30A, K61A, E86A, K97A, H121A, K126A, K146A, E161A, E163A, E170A, E182A, and R201A. Mutant proteins were extracted from cell lysates in an overnight culture and the activity was compared to WT BamHI. Normal enzyme activity was assayed in NEB2 buffer with or without 5% glycerol, while star activity was determined in NEB2 with 39.2% glycerol, though initially, lower percentage glycerol could be used. The substrate used for different reactions was pBR322, pUC19 or lambda DNA. The cleavage reaction was performed at 37° C. for 30 min or 1 hour. It was found that mutants K97A, H121A, K126A, E161A, E182A, R201A were inactive (less than 1% of the WT BamHI activity) while E28A, K146A, E163A, E170A mutants had a similar level of activity including star activity to that of WT enzyme. Three mutants K30A, E86A and K126A were found to have significantly reduced star activity compared with WT BamHI. It was also found that K30A and E86A had similar overall cleavage activity to the WT enzyme while showing significant reduction in star activity. In contrast, K126A had only 25% of the overall cleavage activity of the WT enzyme and less significant improvement on star activity than observed for K30A an E86A.

A recheck on the pUC18-bamHIR plasmid revealed that the normal high copy plasmid had mutated to a low copy plasmid. A pair of primers was designed to transfer the bamHIR gene into the high copy plasmid:

(SEQ ID NO: 67)
5′GGTGGTGCATGCGGAGGTAAATAAATGGAAGTAGAAAAAGAGTTTATT
ACTGAT3′
(SEQ ID NO: 68)
5′GGTGGTGGTACCCTATTTGTTTTCAACTTTATCTTTCCATTTCTTAAT
TGA3′

The template was pUC18-bamhIR WT, with mutations at K30A, E86A or K126A. The PCR composition contained: 5 μl template, 2 μl primers each, 400 μM dNTP, 10 μl 10× Thermopol buffer, 4 units 2 μl Deep Vent™ polymerase, 72 μl H₂O with 0, 2, 6 μl MgSO₄. The PCR conditions were 94° C. for 5 min, followed by 25 cycles of 94° C. at 30 sec, 55° C. at 30 sec and 72° C. at 40 sec and a final extension period of 7 min. The PCR product was digested with SphI and KpnI and was ligated to pUC19 with the same pair of enzyme digestion. The ligated product was transformed into competent E. coli-containing pACYC-bamHIM. 26 colonies that contained the pUC19 version of BamHIR K30A and 12 of those that contained E86A were identified and grown. The activity of BamHI from these cultures was checked. All of them were active. Plasmids from five colonies of each mutation were extracted and the BamHIR plasmids from three of each mutation were sequenced. The identity of plasmids pUC19-BamHI(K30A) and pUC19-BamHI(E86A) were confirmed.

Those mutations that were unsuccessful in pUC18-BamHIR were repeated using the pUC19-BamHI(K30A) vector. The PCR mixture contained: 1 μl template and an amplification mixture containing 2 μl primers each, 400 μM dNTP, 10 μl 10× Thermopol buffer, 4 units 2 μl Deep Vent™ polymerase, 76 μl H₂O with 0, 2, 6 μl 100 μM MgSO₄. The PCR condition was 94° C. for 5 min, followed by 25 cycles of 94° C. for 30 sec, 55° C. for 30 sec and 72° C. for 3 min and 30 sec and a final extension period of 7 min. The PCR products were digested by DpnI and transformed to competent E. coli transformed with pACYC-BamHIM. The enzyme activities were checked on pUC19 substrate. The reaction composition was: 3 μl cell extract, 3 μl NEB2, 3 μl 50% glycerol, 0.5 μl 0.5 μg pUC19, 20.5 μl H₂O. Reaction was at 37° C. for 1 hour. K30A/R122A, K30A/R155A and K30A/K193A were inactive. K30A/K52A and K30A/K88A were about 1/10 of the K30A activity. The normal activity of K30A/K106A, K30A/D154A and K30A/D196A were similar to that of K30A BamHI. The comparison of star activity of these three mutants with K30A at the high concentration glycerol (39.2%) showed that K30A/D196A had similar star activity as K30A, K30A/D154A even has more star activity than K30A, and K30A/K106A had less star activity than K30A. Attempts to isolate the K106A mutation of BamHI in the pUC19 vector failed because of cytotoxicity.

The mutation on the K30, E86 and K106 sites was combined using the inverse PCR: K30A/E86A, E86A/K106A, K30A/K106A and K30A/E86A/K106A. K30A/E86A appeared to be the preferred mutant. After purification, the FI was found to be improved for the BamHI mutant by 25% in all NEB buffers.

Further mutagenesis was done on the site of K30 and E86 randomly:

For K30:

(SEQ ID NO: 69)

5′CAAGCATACAATGAAGTTNNNACATCTATTTGTTCACCT3′

(SEQ ID NO: 70)

5′AGGTGAACAAATAGATGTNNNAACTTCATTGTATGCTTG3′
For E86:

(SEQ ID NO: 71)

5′GATATACTTAAACTTNNNAAGAAAAAAAGGTGGTCCG3′

(SEQ ID NO: 72)

5′CGGACCACCTTTTTTCTTNNNAAGTTTAAGTATATCAAG3′

The PCR composition was: 1 μl template (pUC19-BamHIR(K30A) or pUC19-BamHIR(E86A)) and the amplification mixture as described above was used. The PCR was performed at 94° C. 5 min, followed by 25 cycles of 94° C. 30 sec, 55° C. 30 sec and 72° C. 3 min and 30 sec and a final extension period of 7 min. The PCR products were digested by DpnI and transformed into E. coli (pACYC-BamHIM).

Total of 155 colonies were picked on K30 random mutations, and 158 colonies on E86 site. The colonies were grown overnight and made into cell extract. 0.5 μg pUC19 was digested with 1 μl cell extract in NEB 2 buffer with 42.5% glycerol, 37° C. 1 hour. The cell extract with apparent less star activity was re-assayed under 1, 4, 16 fold dilution on 0.5 μg pUC19 in NEB 2 buffer with 39.2% glycerol, 37° C. 30 min. For those mutants observed to have reduced star activity, the corresponding plasmids were extracted and sequenced to confirm the mutation. A total of 3 clones (#12, #66 and #82) contained the K30 mutation, and a total of 33 clones (#5, #15, #16, #19, #29, #47, #51, #55, #56, #58, #61, #69, #71, #73, #76, #82, #86, #88, #93, #94, #97, #98, #100, #104, #107, #113, #117, #118, #129, #132, #136, #139 and #151) were sequenced. After sequencing, #12 and #66 were found to contain the K30G mutation, and #82 the K30N mutation. Surprisingly, all 33 mutations are E86P mutation, just in different codons (CCA, CCT, CCC, CCG). Among these codons, the CCG occurred at the highest frequency in E. coli (clones #98, #136 and #139).

The cell extracts corresponding to K30G, K30N and K30A were serially diluted as 1, 2, 4, 8, 16 and 32 folds, while E86P and E86A were serially diluted 1, 2, 4, 8, 16, 32, 64, 128 and 256 fold. The serially diluted extracts were reacted with 0.5 μg pUC19 in NEB2 with 39.2% glycerol, 37° C. 30 min. Under extreme conditions, E86P appeared to be much superior to other mutants. At up to 32 times fold digestion, there was no significant star activity band. The difference between E86P and the K30 mutants (K30G, K30N and K30A) was so large that it was not additionally necessary to combine any of these mutations in the E86P mutant.

The activity of BamHI(E86P) was determined for 1 μg lambda DNA, substrate (also used for WT BamHI activity determination). The assay was performed in NEB1 buffer at 37° C. for 1 hour.

### 4. Detailed Comparison of BamHI(E86P) and WT BamHI

### A. The Activity of BamHI(E86P) in Different NEB Buffers

The activity of purified BamHI(E86P) was determined in NEB1, NEB2, NEB3, NEB4 and NEB BamHI buffer, using lambda DNA substrate at 37° C. for 1 hour. BamHI(E86P) was most active in NEB1 buffer and NEB2, while having 50%, 50% and 25% activity levels in NEB3, NEB4, and BamHI buffer.

B. A Comparison of Cleavage Activity of BamHI(E86P) and WT BamHI on pUC19

There is one GGATCC site (BamHI site) and 6 AGATCC sites (BamHI star activity site) in pUC19 so that pUC19 was selected as a preferred substrate for comparison of the BamHI(E86P) and WT BamHI.

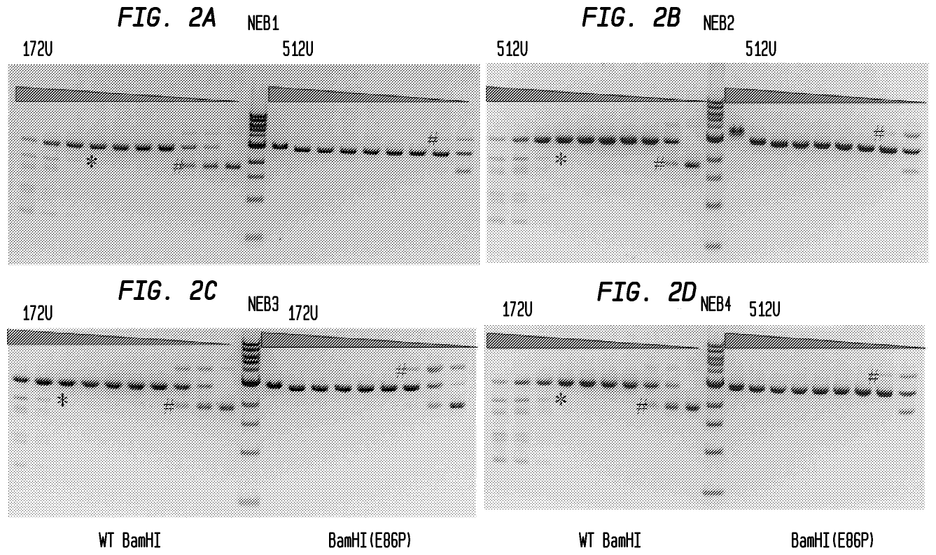

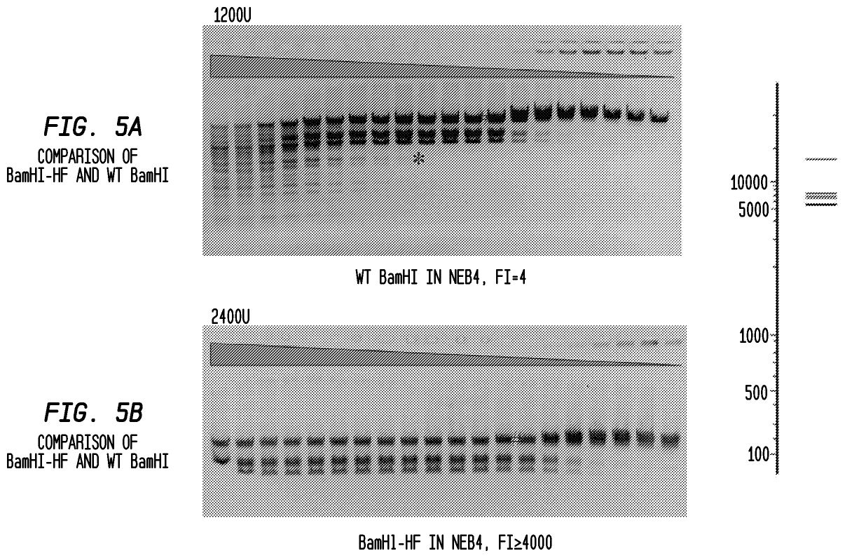

0.5 mg pUC19 was digested by WT BamHI and BamHI(E86P) in a serial dilution of 1, 3, 9, 27, 81, 243, 729, 2181, 6561, and 19683 folds with NEB dilution buffer A, in different buffers. WT BamHI showed star activity in every NEB normal buffer, while BamHI(E86P) showed no star activity bands at all (FIGS. 2-5). This demonstrated that BamHI(E86P) had greatly reduced star activity while retaining the cognate cleavage activity.

### C. A Comparison of Cleavage Activity of BamHI(E86P) and WT BamHI on Lambda DNA Substrate

To calculate the Fidelity Index, the restriction enzyme was diluted with dilution buffer, and the glycerol concentration was kept constantly at 5%. In the standard reaction condition used here, lambda DNA substrate concentration was 1 μg and the total reaction volume was 50 μl. In order to keep the enzyme volume at 10%, the enzyme was added in a volume of 5 μl. This is equivalent to 0.6 μg of substrate digested by 3 μl of restriction enzyme in a total volume of 30 μl. 0.6 mg lambda DNA was digested by 3 μl WT BamHI and BamHI(E86P) in a 1:2 serial dilution from 1 to 32768, in NEB1, NEB2, NEB3, NEB4 and NEB BamHI buffer at 37° C. for 1 hour.

TABLE 5
Fidelity Indices for WT and mutant BamHI in various buffers

| | BamHI(E86P) | | WT BamHI | | |
|---|---|---|---|---|---|
| | | Fidelity | | Fidelity | Improvement |
| Buffer | Activity | Index | Activity | Index | Factor |
| NEB1 | 100% | ≥4000 | 50% | 4 | ≥1000 |
| NEB2 | 100% | ≥4000 | 100% | 16 | ≥250 |
| NEB3 | 50% | ≥4000 | 100% | 32 | ≥125 |
| NEB4 | 50% | ≥4000 | 50% | 4 | ≥1000 |
| BamHI | 25% | ≥2000 | 50% | 32 | ≥125 |
| buffer | | | | | |

### 5. Further Improvement of BamHI for High Fidelity Mutants

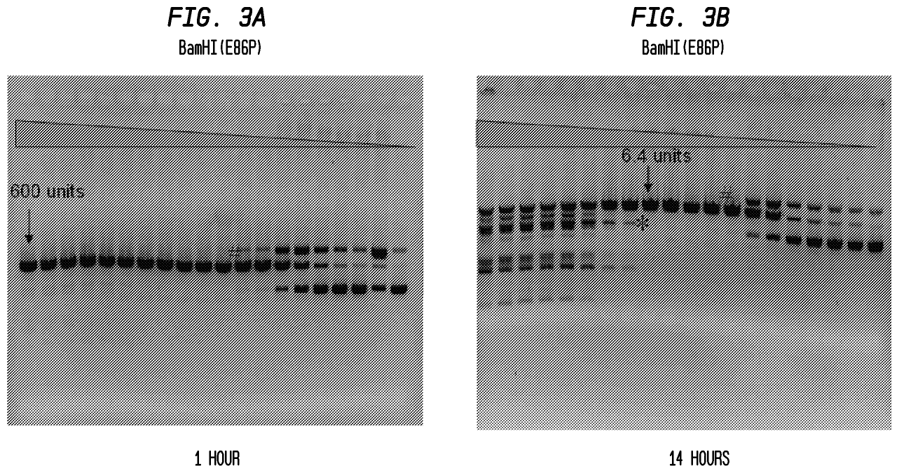

At one hour level, the BamHI(E86P) appeared to be a good high fidelity BamHI mutant. However, when the reaction time was extended (e.g. overnight, or 14 hours), star activity bands appeared even though the star activity of E86P was not detected at one hour. (FIG. 3) The search for improved high fidelity BamHI was continued.

### 6. Mutations of Other Charged and Polar Residues

The other charged residues (Arg, Lys, His, Asp, Glu) were mutated to Ala at the positions of 2, 4, 5, 6, 10, 11, 13, 14, 18, 19, 20, 43, 51, 62, 69, 70, 76, 77, 78, 81, 87, 89, 94, 98, 101, 104, 107, 111, 113, 132, 133, 135, 137, 160, 167, 200, 204, 205, 207, 208, 209, 211, 213 in SEQ ID NO:19. The mutations were done on the template of pUC19-BamHI(K30A).

Other polar residues (Ser, Thr and Tyr) were mutated to Ala while Tyr was mutated to Phe at the positions of 9, 17, 26, 32, 36, 41, 42, 44, 46, 50, 65, 66, 71, 72, 75, 96, 103, 114, 118, 119, 123, 150, 151, 153, 157, 165, 169, 184, 186, 195, 199, 202 in SEQ ID NO:19.

By using similar mutation and screen methods, the following mutations were discovered to have reduced star activity, K30A/K87A, E86P/K87E, E86A/Y165F, and K30A/E167A. E86P/K87E was identified as a mutant with improved properties in the presence of additional DMSO. However, the activity of this mutant in normal reaction buffer was much lower than that of WT BamHI.

The following combination of mutations was made: E86P/Y165F, E86P/E167A, E86P/Y165F/E167A, K30A/Y165F/E167A, K30G/Y165F/E167A, K30A/Y165F/E167A, E86A/Y165F/E167A. All had low activity.

Up to this point, it was found that E167A and Y165F had a strong effect, K87A had medium effect, and K30A and E86A had weak effect on the BamHI star activity. E86P is a special mutation that reduces star activity at 1 hour level but not overnight.

### 7. Mutation of E167 and Y165 to all Other Residues

E167 was mutated to all other residues in pUC19-BamHI by changing the codon to GCA for Ala, TGC for Cys, GAC for Asp, TTC for Phe, GGT for Gly, CAC for His, ATC for Ile, AAA for Lys, CTG for Leu, ATG for Met, AAC for Asn, CCG for Pro, CAG for Gln, CGT for Arg, TCC for Ser, ACC for Thr, GTT for Val, TGG for Trp, and TAC for Tyr.

After comparison of all the mutants, the E167T mutation was preferred, while E167R, E167K, E167L and E1671 mutations showed improvement in reduced star activity compared with E167A.

Y165 was also mutated to all other amino acid residues by changing the corresponding codon to GCT for Ala, TGC for Cys, GAC for Asp, GAA for Glu, GGT for Gly, CAC for His, ATC for Ile, AAA for Lys, CTG for Leu, ATG for Met, AAC for Asn, CCG for Pro, CAG for Gln, CGT for Arg, TCC for Ser, ACC for Thr, GTT for Val, TGG for Trp.

After comparison of all the mutants, the presence of Y165F resulted in significant cleavage activity while other mutations of listed immediately above showed low activity or no cleavage activity.

### 8. Further Mutations on BamHI(E167T)

All charged and polar residues were mutated to Ala, on the template of puc19-BamHI(E167T), as the same procedure as above.

E163A/E167T as the preferred mutation was identified as BamHI-HF.

### 9. Comparison of BamHI-HF to WT BamHI

Introduction of a mutation at E163 resulted in reduced thermostability of the BamHI mutant, as did mutation P173A when added to other mutations responsible for reducing star activity.

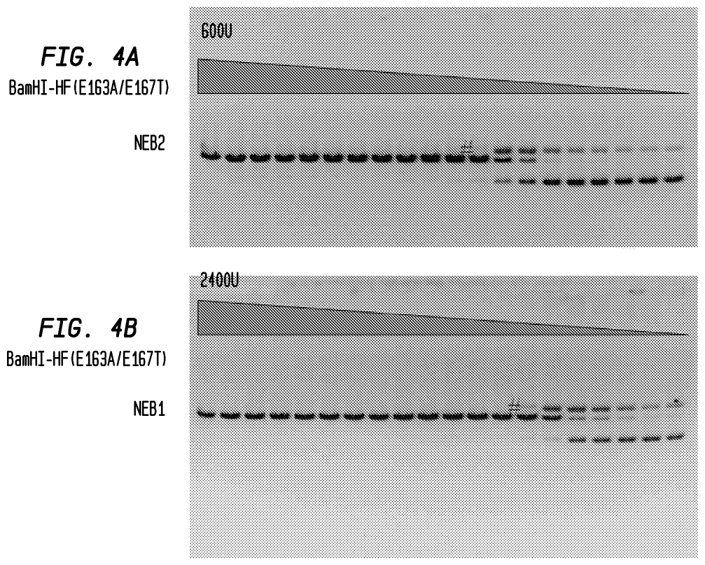

BamHI-HF, unlike the BamHI(E86P), had no significant star activity in an overnight reaction in NEB1-4 buffers. FIG. 4 shows the results in NEB1 and NEB2. Hence BamHI(E163A/E167T) was selected as the preferred high fidelity BamHI.

The fidelity indices of BamHI-HF were measured in all of the four NEB buffers on lambda DNA substrate, with diluent A, at 37° C. and compared with the WT enzyme.

TABLE 6
Comparison of BamHI-HF and WT BamHI

| | BamHI-HF | | WT BamHI | | |
|---|---|---|---|---|---|
| | | Fidelity | | Fidelity | Improvement |
| Buffer | Activity | Index | Activity | Index | Factor |
| NEB1 | 100% | ≥8000 | 50% | 4 | ≥1000 |
| NEB2 | 50% | ≥4000 | 100% | 16 | ≥250 |
| NEB3 | 12.5% | ≥250 | 100% | 32 | ≥8 |
| NEB4 | 50% | ≥4000 | 50% | 4 | ≥1000 |

BamHI-HF has a highest activity in NEB1, the fidelity index is ≥8000, WT BamHI has the highest activity in NEB2 and NEB3, and the highest FI is 32. The overall FI improvement factor, which is the ratio of the FI in the best buffer for each of the mutant and the WT enzyme, is ≥8000/32=250 fold.

### 10. Additional Mutations of BamHI

E163A/E167T/P173A was predicted to have a preferred reduction in star activity and additionally to be thermolabile.

(E86P/K87S/K88P/E163S/E170T/P173A) was tested. This mutant displayed 10-fold reduction in specific activity but had a compensating increased yield of protein from host cells.

Other BamHI mutants that shared reduced thermostability, reduced star activity and acceptable specific activity include:
E86P/K87R/K88G/E163 S/E170T/P173A
E86P/K87P/K88R/E163S/E170T/P173A/E211K
E86P/K87T/K88R/E163S/E170T/P173A/N158S
E86P/K87S/K88P/E163 S/E170T/P173A
E86P/K87G/K88S/E163S/E170T/P173A
E86P/K87R/K88Q/E163 S/E170T/P173A

### Example 2: Preparation of a High Fidelity EcoRI

### 1. Expression of EcoRI

PCR on EcoRI used the following primers:

(SEQ ID NO: 73)
GGTGGTGCATGCGGAGGTAAATAAATGTCTAATAAAAAACAGTCAAATAG
GCTA
(SEQ ID NO: 74)
GGTGGTGGTACCTCACTTAGATCTAAGCTGTTCAAACAA

The PCR product was then digested with a second pair of restriction endonucleases—SphI and Acc65I, and ligated into the pUC19 digested with the same second pair of restriction endonucleases. The ligated plasmid was then be transformed into competent E. coli premodified with pACYC-MlucIM.

### 2. Mutagenesis of EcoRI

Initial selection of target amino acid residues resulted from a comparison of EcoRI with its isoschizomer RsrI, which is also known for its star activity.

Except for D91, E111 and K113, which were known active center residues, the 42 charged residues were identical or similar in the two endonucleases. The charged residues were as follows: K4, R9, K15, K29, H31, D32, E37, E49, R56, R58, K63, E68, K71, D74, K89, E96, K98, K99, R105, H114, D118, K130, D133, D135, E144, R145, H147, K148, E152, E160, H162, E170, E177, R183, D185, R200, D202, R203, E253, R264, D269.

All of these charged residues were mutated to Ala (codon GCA, GCT, GCC or GCG) and the mutated genes amplified and cloned as follows:

The amplification mixture was the same as used in Example 1 (2 μl PCR primers each, 400 mM dNTP, 4 units of Deep Vent DNA polymerase, 10 μl 10× Thermopol buffer with additional 0, 2, 6 μl MgSO₄, and the total reaction volume was 100 μl) and was added to 1 μl pUC19-EcoRI).

The PCR reaction conditions was 94° C. for 5 min, followed by 25 cycles of 94° C. for 30 sec, 55° C. for 30 sec, 72° C. for 3 min and 30 sec and a final extension time at 72° C. for 7 min. After PCR, the product was purified by the standard Qiagen spin column (Qiagen, Valencia, CA). 16 μl PCR product was digested by 20 units of DpnI for 1 hour. The digested product was transformed into a methylase protected competent E. coli preparation.

### 3. Screening EcoRI High Fidelity Mutants

Three colonies each were picked for each mutation and grown in LB with Ampicillin and Chloramphenicol for overnight. The activity assay was performed on pBR322 and lambda DNA to ensure the mutant had at least similar activity to WT EcoRI. Then these mutants were tested using 3 μl of cell extract in 2-fold serial dilution, 12 μl 50% glycerol, 3 μl of NEB1 buffer, 0.5 μl pBR322 and 11.5 μl water, reacted at 37° C. for one hour. However, none of the mutations improved the performance of star activity.

From this result, it was concluded that an effective mutation could not always be recognized as a homologous residue between isoschizomers.

### 4. Repeat Mutagenesis on the Rest of 32 Charged Residues

All remaining 32 charged residues were mutated into Ala as described in step 2 by targeting amino acid residues 5, 12, 14, 26, 40, 43, 44, 59, 62, 65, 72, 76, 100, 103, 117, 123, 131, 192, 221, 225, 226, 227, 228, 242, 244, 245, 247, 249, 257, 268, 272 and 277.

The numbers above correspond to amino acid positions in the EcoRI protein sequence (SEQ ID NO:83).

### 5. Repeat Selection

Four colonies were picked from each sample containing a different type of mutation and grown in 4 ml LB with CAM. After sonication, cell extracts were tested on lambda DNA substrate in normal glycerol condition in NEB1 buffer. Those extracts with similar activity were tested again on pUC19 substrate by adding 3 μl of cell extract in two-fold serial dilutions, in 3 μl of NEB2 buffer to 0.5 μl of pUC19 and 23.5 μl 50% glycerol to provide a final concentration of 39.2% glycerol in the reaction mixture.

Among all of these mutants, K62A was found to be the mutation with the least star activity and a high FI. R9A, K15A, R123A, K130A, R131A, R183A mutants all showed partial reduction in star activity. Interestingly, one clone containing the targeted mutation K5A showed a partial improvement. Additionally, a secondary mutation, S2Y was found after sequencing. Separation of these two mutations revealed that the effective mutation for this isolate was S2Y. D135A and R187A EcoRI also had much less star activity. However, the cleavage activity of these mutants was not optimal.

6. Comparison of EcoRI(K62A) with WT EcoRI

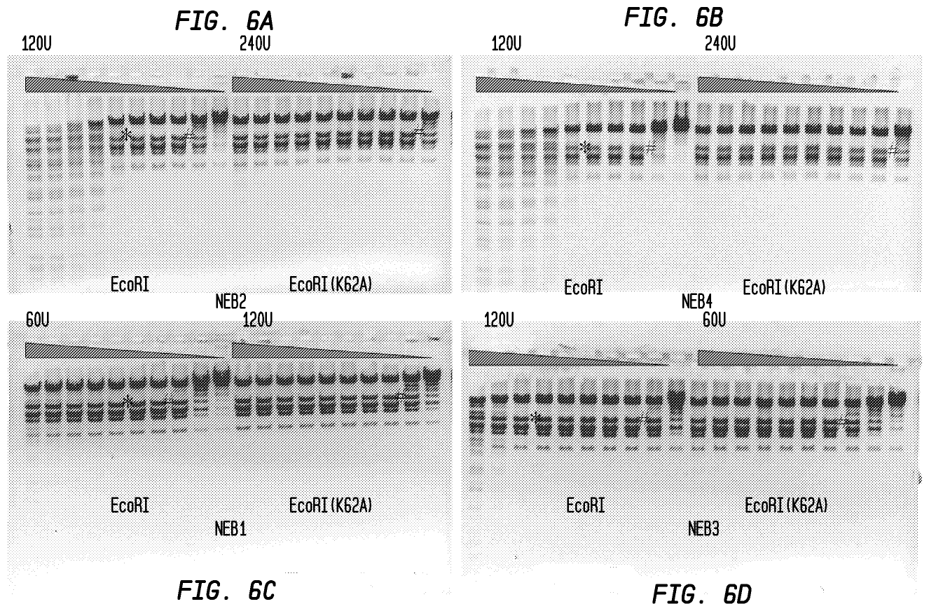

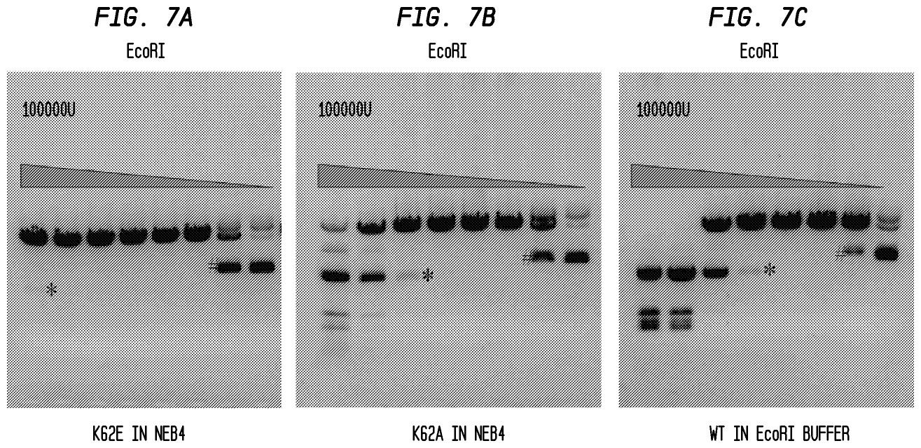

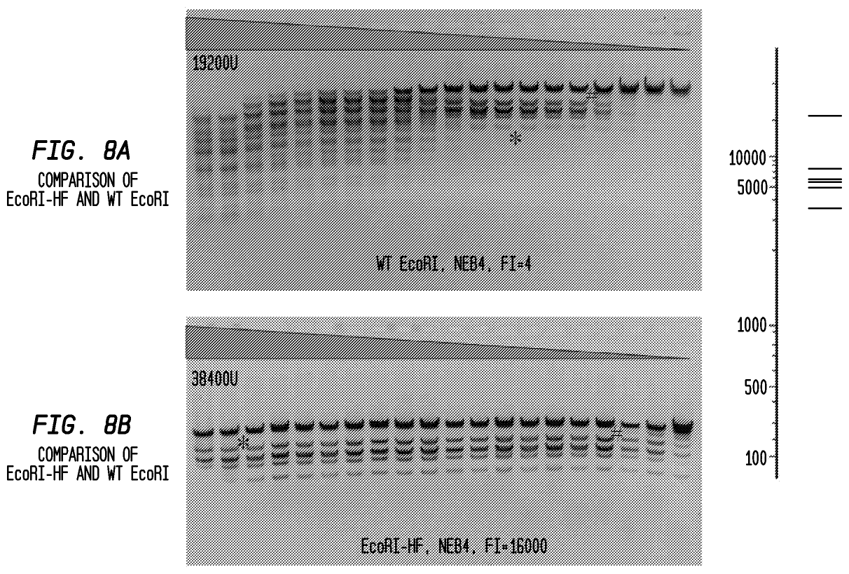

A side-by-side comparison was performed in a 3-fold serial dilution using NEB dilution buffer C, by digesting 0.6 μg of lambda DNA in four different NEB buffers (FIG. 6). EcoRI(K62A) had substantially less star activity than the WT EcoRI.

A more quantitative comparison was done by determining the Fidelity Index measurement for EcoRI(K62A) and WT EcoRI. The conditions for the fidelity index measurement was the same as for Table 2 using lambda DNA as substrate and, dilution buffer C. The reaction was incubated at 37° C. for 1 hour and the digestion products analyzed on an 0.8% agarose gel.

TABLE 7
Fidelity Index for EcoRI(K62A) and WT EcoRI

| | | EcoRI(K62A) | | WT EcoRI | | |
|---|---|---|---|---|---|---|
| | | | Fidelity | | Fidelity | Improvement |
| | Buffer | Activity | Index | Activity | Index | Factor |
| 7. | NEB1 | 50% | 32000 | 50% | 250 | 128 |
| Further | NEB2 | 50% | 8000 | 100% | 4 | 2000 |
| mutation | NEB3 | 12.5% | 4000 | 100% | 250 | 16 |
| of | NEB4 | 100% | 32000 | 100% | 4 | 8000 |
| EcoRI | EcoRI | 12.5% | 4000 | 100% | 250 | 16 |
| | buffer | | | | | |

Though it was not apparent that the EcoRI(K62A) had star activity on lambda DNA substrate, star activity was observed using Litmus28 substrate after a 10 hours digestion. EcoRI(K62A) in NEB4 had significantly reduced star activity compared with WT EcoRI in EcoRI buffer (FIG. 7).

Further improvements were investigated. EcoRI(K62) was mutated to all other amino acid residues by changing K to the corresponding codons as in the example 1. K62S and K62L were similar as K62A. EcoRI(K62E) had a ≥100 fold overall fidelity index improvement factor when compared with EcoRI(K62A) as shown in FIG. 6. EcoRI(K62E) was named EcoRI-HF.

### 8. Comparison of EcoRI-HF and WT EcoRI

A quantitative comparison was done by the FI measurement on EcoRI-HF and WT EcoRI in diluent C. The conditions for the FI measurement were the same as in Table 2 using lambda DNA as substrate. The reaction conditions were 37° C. for 1 hour and the results analyzed on a 0.8% agarose gel (FIG. 8).

TABLE 8
Comparison of EcoRI-HF and WT EcoRI

| | EcoRI-HF | | WT EcoRI | | |
|---|---|---|---|---|---|
| | | Fidelity | | Fidelity | Improvement |
| Buffer | Activity | Index | Activity | Index | Factor |
| NEB1 | 12.5% | 2000 | 50% | 250 | 8 |
| NEB2 | 100% | 4000 | 100% | 4 | 1000 |
| NEB3 | 0.4% | 250 | 100% | 250 | 1 |
| NEB4 | 100% | 16000 | 100% | 4 | 4000 |
| EcoRI buffer | 0.4% | 250 | 100% | 250 | 1 |

The overall fidelity index improvement factor was found to be 64 fold (16000 in NEB4 for EcoRI-HIF to 250 of WT EcoRI in NEB3).

### Example 3: Engineering a High Fidelity ScaI

### 1. Expression of ScaI

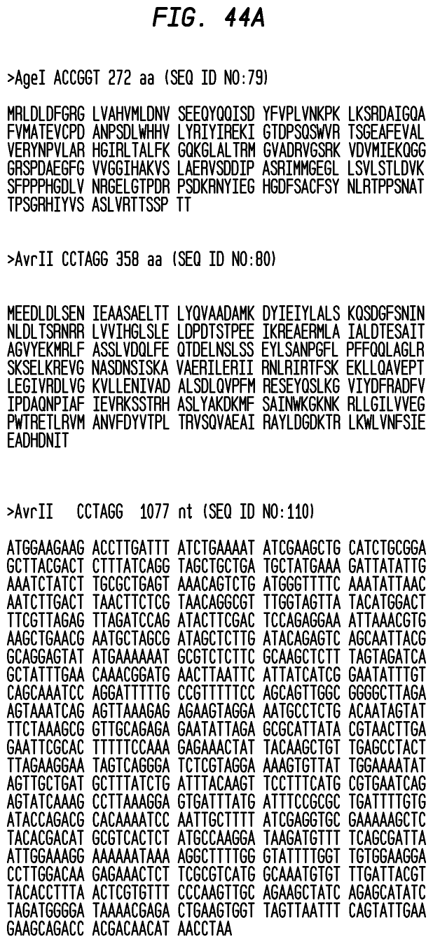

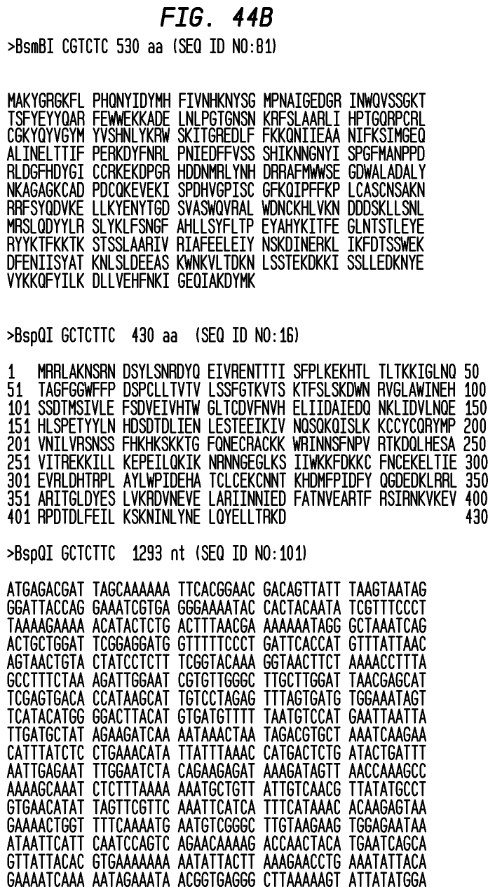

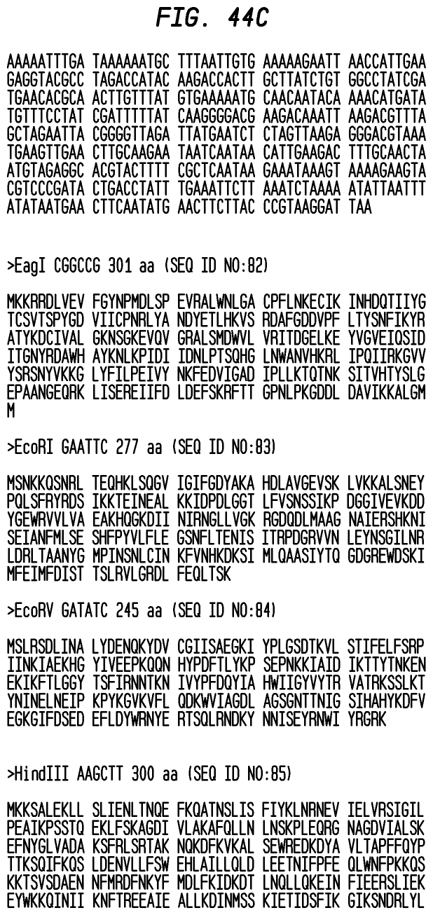

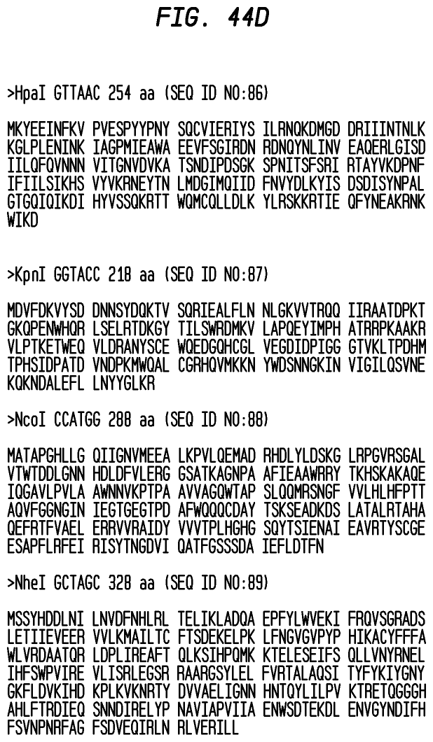

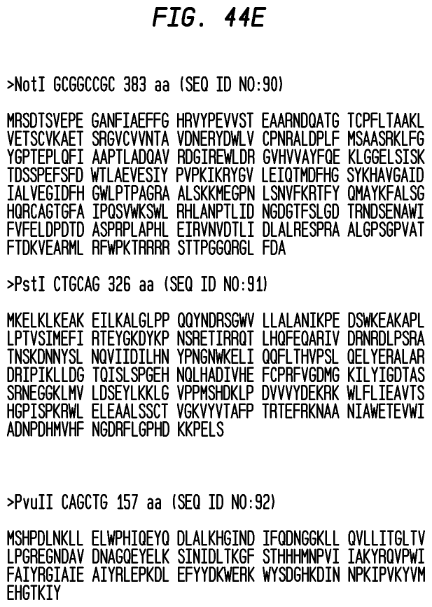

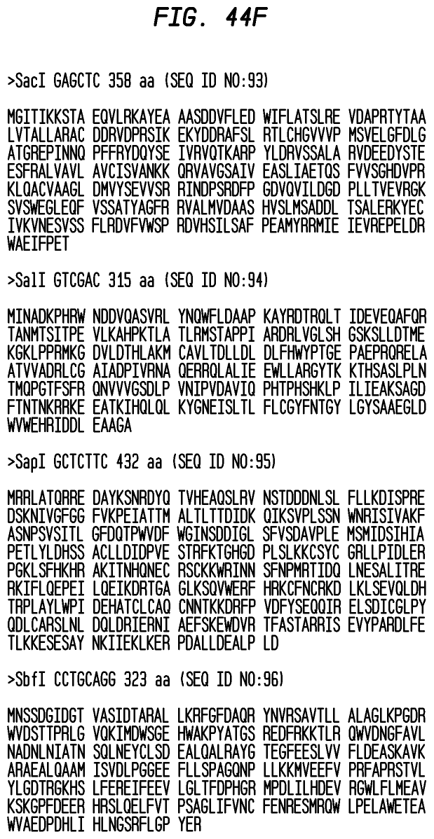

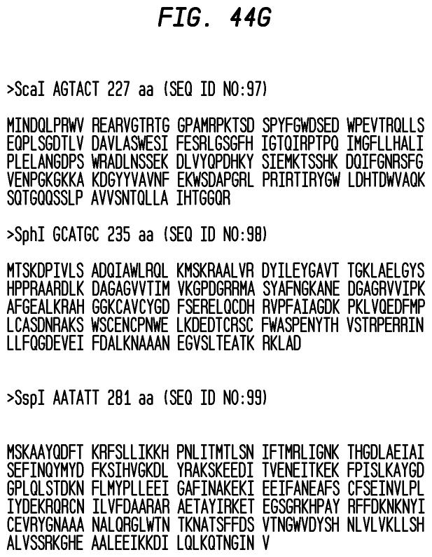

The sequence for ScaI restriction endonuclease and methylase are described in REBASE and in GenBank and is presented in FIG. 44G (SEQ ID NO:97). The genes expressing these enzymes were inserted into plasmids to produce pRRS-ScaI and pACYC184-ScaIM respectively. pACYC184-ScaIM was then transformed into competent E. coli host cells. The pRRS vector was derived from pUC19 and differs only in the presence of multiple cloning sites. pRRS-ScaIR was transformed into E. co/i (pACYC-ScaIM) to make an expression strain. Plating and cell culture were performed at 30° C.

### 2. Mutagenesis of ScaI

ScaI has two sequenced isoschizomers: LlaDI and NmeSI. However, there was no known information on the star activity of LlaDI or NmeSI nor was there any information on the active site of these enzymes. Therefore all 58 charged residues were initially selected for targeted mutation at positions 4, 8, 11, 12, 14, 18, 25, 27, 30, 37, 39, 40, 43, 46, 51, 57, 61, 68, 72, 74, 80, 86, 97, 103, 108, 112, 114, 119, 120, 121, 127, 128, 129, 133, 135, 139, 140, 141, 147, 152, 156, 158, 159, 161, 162, 171, 172, 175, 179, 182, 184, 187, 172, 175, 192, 193, 195, 200, 222, 227 in the protein.

The numbers above correspond to amino acid positions in the ScaI protein sequence (SEQ ID NO:97).

The method of primer design and PCR is similar to that described in Example 1 for BamHI and Example 2 for EcoRI. Mutagenesis was achieved by varying annealing temperature and DNA polymerases. The PCR product was digested with DpnI and transformed into competent E. coli (pACYC184-ScaIM).

### 3. Selection of ScaI High Fidelity Mutants

Four colonies from each mutant of ScaI mutant were picked and grown in 4 ml LB with 100 μg/ml Amp and 33 μg/ml Cam at 30° C. overnight. Each cell culture was sonicated and the activity tested on lambda DNA in NEB2 buffer. Those that were apparently active were retested in 10, 100, and 1000 fold dilutions. Since ScaI has very significant star activity, the star activity bands were easily compared for the mutants versus the WT restriction endonuclease. Those with reduced star activity were retested with a two-fold serial dilution with NEB dilution buffer A. The FI was measured for each of the mutants. The FI was ⅛ for WT ScaI in NEB 2 buffer. Four mutants with similar activity levels were found to have greatly reduced star activity compared with WT ScaI. Mutant #6-3 ScaI had two fold more activity and the FI was 4 or 32 times better than WT ScaI. #26-2 ScaI has two fold more activity and an FI which was 8 or 64 times better than WT; #28-2 ScaI has 2 fold more activity and FI to be 120 or 1000 times better than WT, #54-3 has same activity as WT and FI to be 250 or 2000 times better than WT.

Four mutants: #6-3, #26-2, #28-2 and #54-3 ScaI were further tested in the presence of 36.7% glycerol for digestion of lambda DNA substrate. #54-3 showed a greater improvement in reduced star activity than the other three mutants.

After the plasmid was extracted, #6-3 was sequenced and found to have a mutation at R18A. #26-2 was sequenced and found to have a mutation at Ri 12A. #28-2 was sequenced and found to have a mutation at E119A. These mutations were predicted. However, the #54-3 was found to have a double mutant-H193A/S201F. The S201F was a spontaneously secondary mutation that occurred during PCR, and was located outside the primer region of the H193A mutation.

To understand which residue was primarily responsible for the reduction in star activity a single mutation (S201F) was introduced into ScaI using the following primers:

| (SEQ ID NO: 77) | |
|---|---|
| | 5′-GATTGGGTGGCGCAGAAATTTCAAACGGGCCAGCAGTCG-3′ |
| (SEQ ID NO: 78) | |
| | 5′-CGACTGCTGGCCCGTTTGAAATTTCTGCGCCACCCAATC-3′ |

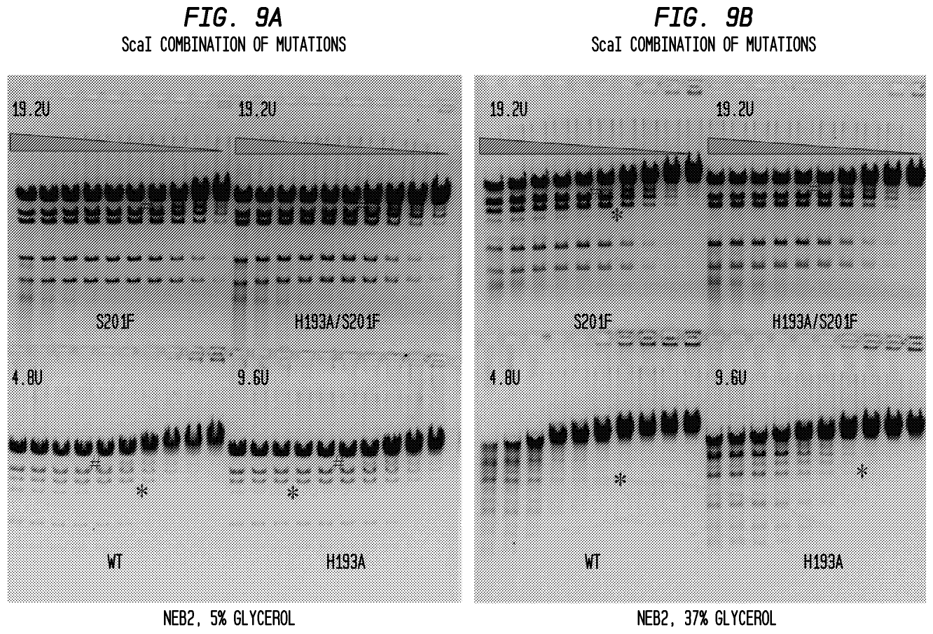

The sequences for ScaI(H193A), ScaI(S201F) and ScaI(H193A/S201F) were confirmed. The three mutants and the WT ScaI were compared at the glycerol level of 5% and 37% (FIG. 9). S201F contributed significantly to the FI in contrast to H193A, which only contributed weakly. However, these two mutations appeared to be additive in improving the FI. S201F did not show star activity in 5% glycerol, but did show some star activity in 37% glycerol. H193A had some star activity in 5% glycerol, and significant star activity in 37% glycerol. However, with the combination of these two mutations, no star activity was detected in either 5% or 37% glycerol. This finding not only shows that the amino acids with hydroxyl group can be major active residue for star activity, but also that the right combination of the mutations can push the improvement in fidelity to a very high level. If mutations of charged residues fail to improve star activity, it is here observed that mutations on Ser, Thr and Tyr can be successful in improving the fidelity index. The ScaI(H193A/S201F) was labelled ScaI-HF.

### 4. Comparison of ScaI-HF and WT ScaI

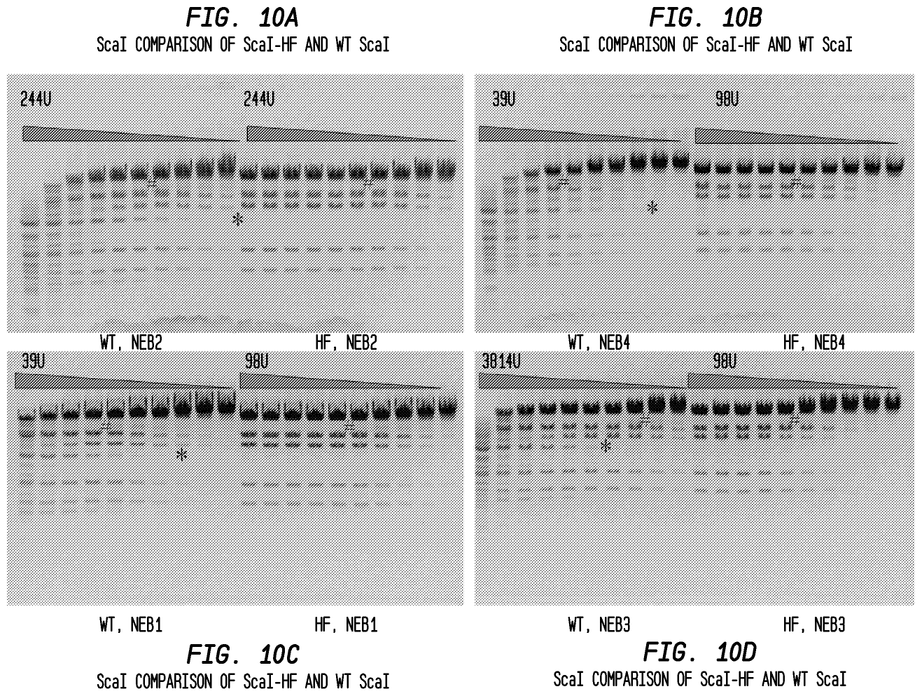

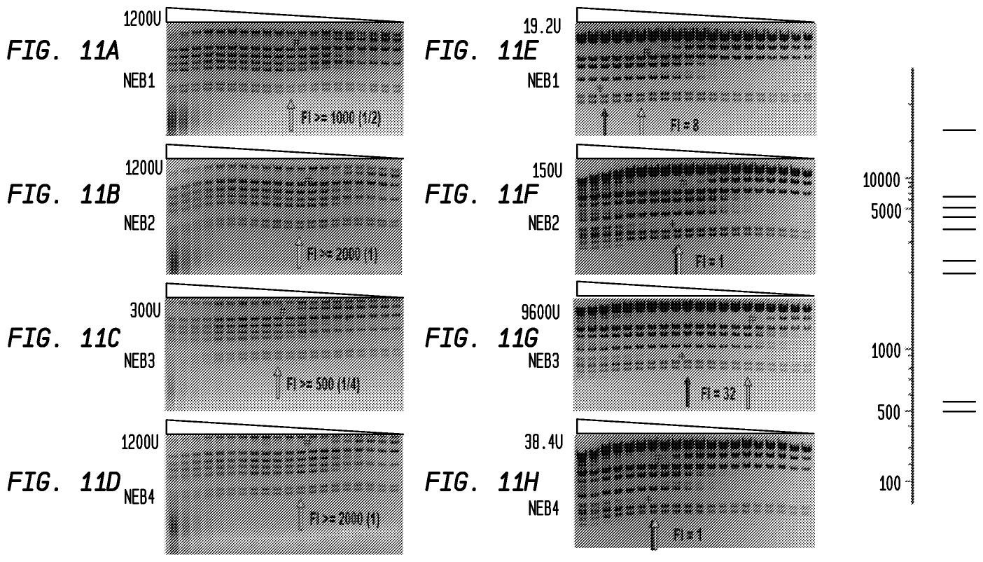

ScaI-HF and WT ScaI were compared at a 2.5 fold serial dilution with NEB dilution buffer A in different NEB buffers on 1 μg lambda DNA in four different NEB buffers, 37° C., 1 hour (FIG. 10).

TABLE 9
Comparison of Fidelity Index for ScaI-HF and WT ScaI

| | ScaI-HF | | WT ScaI | | |
|---|---|---|---|---|---|
| | | Fidelity | | Fidelity | Improvement |
| Buffer | Activity | Index | Activity | Index | Factor |
| NEB1 | 12% | 250 | 6% | 1/64 | 16000 |
| NEB2 | 100% | 120 | 100% | ⅛ | 2000 |
| NEB3 | 3% | 2000 | 25% | 4 | 500 |
| NEB4 | 100% | 250 | 1% | 1/32 | 8000 |

ScaI-HF performed best in NEB2 and NEB4 buffers, in which the best FI was 250; WT ScaI performed best in NEB2 buffer, in which the FI was ⅛. The overall FI improvement factor was 250/(⅛)=4000.

### Example 4: Engineering of High Fidelity SalI

### 1. Expression of SalT

SalI was expressed in E. coli transformed with placzz1-SalIR and pACYC-Hpy166IIM where placzzI is a pUC19 plasmid which utilizes the lac promoter to express the restriction endonuclease gene that is inserted into an adjacent multi-copy site. Hpy166IIM protects the outside four bases of SalI.

### 2. Mutagenesis of SalI

86 charged residues of SalI were mutated to Ala using the similar PCR methods in the previous examples: 5, 6, 8, 9, 12, 13, 19, 27, 31, 34, 35, 37, 42, 43, 45, 50, 60, 63, 65, 67, 73, 82, 83, 84, 90, 93, 97, 100, 101, 103, 107, 109, 111, 114, 116, 119, 126, 129, 131, 134, 140, 143, 145, 147, 148, 156, 157, 164, 168, 172, 173, 174, 180, 181, 186, 190, 191, 193, 210, 218, 226, 232, 235, 237, 238, 244, 246, 250, 256, 257, 258, 259, 260, 261, 264, 266, 271, 275, 297, 300, 304, 305, 306, 308, 309, 311.

The numbers above correspond to amino acid positions in the SalI protein sequence (SEQ ID NO:94).

The mutants were grown in LB with Amp and Cam at 30° C. overnight.

### 3. Selection of SalI-HF

The selection of SalI-HF was performed as described in the previous examples. The major difference was that the star activity of SalI could not be easily assayed in the crude extract, either in 5% glycerol or high glycerol concentration. Glycerol not only promoted the star activity of SalI, but also greatly inhibited the cognate activity.

Active mutants were assayed in both 5% glycerol and 37% glycerol on HindIII digested lambda DNA. The mutants #22, #26, #29, #31, #43 and #51 were tested for cleavage activity in all four NEB buffers. After several rounds of comparison in different conditions and substrates, #31, SalI(R107A) was found to be the preferred mutant, retaining high cleavage high activity, but displaying substantially reduced star activity. SalI(R107A) was labeled SalI-HF.

### 4. Comparison of SalI-HF and WT SalI

The FI of SalI-HF and WT SalI were determined (FIG. 11). The results are shown as Table 10 (below):

TABLE 10
Comparison of SalI-HF and WT SalI

| | SalI-HF | | WT SalI | | |
|---|---|---|---|---|---|
| | | Fidelity | | Fidelity | Improvement |
| Buffer | Activity | Index | Activity | Index | Factor |
| NEB1 | 50% | ≥1000 | 0.2% | 8 | 16000 |
| NEB2 | 100% | ≥2000 | 6% | ⅛ | 2000 |
| NEB3 | 25% | ≥500 | 100% | 4 | 500 |
| NEB4 | 100% | ≥2000 | 0.8% | 1/32 | 8000 |

SalI-HF performed best in NEB 2 and NEB 4 buffers, in which both FIs are ≥2000; WT SalI performed best in NEB 3 buffer, in which the FI was 4. The overall FI improvement factor was ≥2000/4=≥500.

### Example 5: Engineering of High Fidelity SphI

### 1. Expression of SphI

SphI was expressed in E. coli (placzz1-SphIR, pACYC184-CviAIIM). CviAIIM protects the internal four bases of SphI. The transformed cells were grown in LB with Amp and Cam at 37° C. overnight.

### 2. Mutagenesis of SphI

All charged residues in SphI were mutated to Ala using the methods described in the Example 1 to Example 4. A total of 71 mutations were made: 3, 5, 12, 18, 21, 24, 25, 30, 31, 35, 43, 46, 51, 54, 57, 58, 60, 61, 72, 75, 77, 78, 87, 90, 91, 95, 100, 104, 107, 108, 110, 113, 120, 123, 124, 125, 129, 130, 131, 139, 140, 142, 146, 147, 155, 157, 159, 164, 170, 172, 173, 175, 178, 184, 186, 190, 194, 196, 197, 198, 206, 207, 209, 212, 215, 221, 227, 230, 231, 232, 235.

The numbers above correspond to amino acid positions in the SphI protein sequence (SEQ ID NO:98).

### 3. Selection of SphI-HF

Four colonies of each mutation were grown up in LB with Amp and Cam at 37° C. overnight. The activity selection was mainly on pBR322 in 5% glycerol and 30% glycerol in NEB2. With the experience of previous examples, the selection of high fidelity SphI was straightforward. SphI mutants D91A, K100A, D139A and D164A were found to significantly reduce star activity in SphI. Among them, K100A was the preferred mutation with the least star activity. SphI(K100A) was named as SphI-HF.

### 4. Comparison of SphI-HF and WT SphI

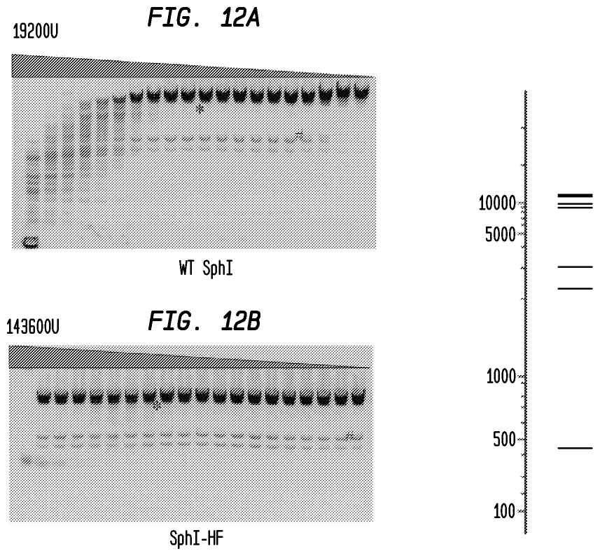

The comparison of SphI-HF and WT SphI was done side by side in their respective preferred buffers. SphI-HF was 2-fold serial diluted with NEB dilution buffer A and reacted in NEB4, and WT SphI was 2-fold serial diluted with NEB dilution buffer B. The digestion on lambda DNA is compared in FIG. 12.

TABLE 11
FI comparison of SphI-HF and WT SphI

| | SphI-HF | | WT SphI | | |
|---|---|---|---|---|---|
| | | Fidelity | | Fidelity | Improvement |
| Buffer | Activity | Index | Activity | Index | Factor |
| NEB1 | 50% | ≥1000 | 100% | 64 | ≥16 |
| NEB2 | 50% | ≥1000 | 100% | 32 | ≥32 |
| NEB3 | 3% | ≥120 | 25% | 64 | ≥2 |
| NEB4 | 100% | ≥2000 | 50% | 16 | ≥64 |

SphI-IF performed best in NEB4, in which FI is ≥2000; WT SphI performed best in NEB1 or NEB2, in which the preferred FI is 64. The overall FI improvement factor was ≥32.

### Example 6: Engineering of High Fidelity PstI

### 1. Expression of PstI

PstI was expressed from E. coli(pACYC-HpyCH4VM, pPR594-PstIR). HpyCH4VM protects the internal four bases of PstI. pPR594 is a expression vector with Amp resistance and ptac promoter. The cell was grown in LB with Amp and Cam at 30° C., the culture was then induced by IPTG overnight.

### 2. Mutagenesis of PstI

92 charged residues were mutated to Ala using the method described in the previous examples. These were: 8, 10, 11, 14, 25, 26, 38, 40, 41, 44, 45, 47, 58, 61, 63, 66, 67, 69, 73, 74, 77, 78, 82, 85, 88, 91, 92, 94, 95, 99, 104, 105, 116, 119, 127, 128, 136, 142, 145, 146, 150, 151, 152, 156, 159, 169, 170, 174, 176, 179, 180, 184, 188, 191, 197, 202, 204, 207, 212, 214, 217, 218, 226, 227, 228, 231, 236, 237, 238, 239, 240, 246, 251, 257, 258, 261, 263, 273, 282, 284, 286, 287, 295, 297, 302, 305, 306, 309, 313, 314, 319 and 320.

The numbers above correspond to amino acid positions in the PstI protein sequence (SEQ ID NO:91).

After the PCR products were digested with DpnI, the samples were transformed into competent E. coli(pACYC-HpyCH4VM) and grown on LB plate with Amp and Cam.

### 3. Selection of PstI-HF

The selection of PstI-HF was similar to the previous samples. The normal activity enzyme activity was tested on lambda DNA with 5% glycerol, and the star activity was tested on pBR322 substrate in the condition of NEB4 buffer and 20% DMSO. DMSO enhanced the star activity more significantly than the same concentration glycerol. During the selection, #26, #56 and #65 had reduced star activity compared to the WT. When each was sequenced, the mutations were found to be D91A, E204G and K228A/A289V. Mutant #26 PstI(D91A) was labeled PstI-HF.

### 4. Comparison of PstI-HF and WT PstI

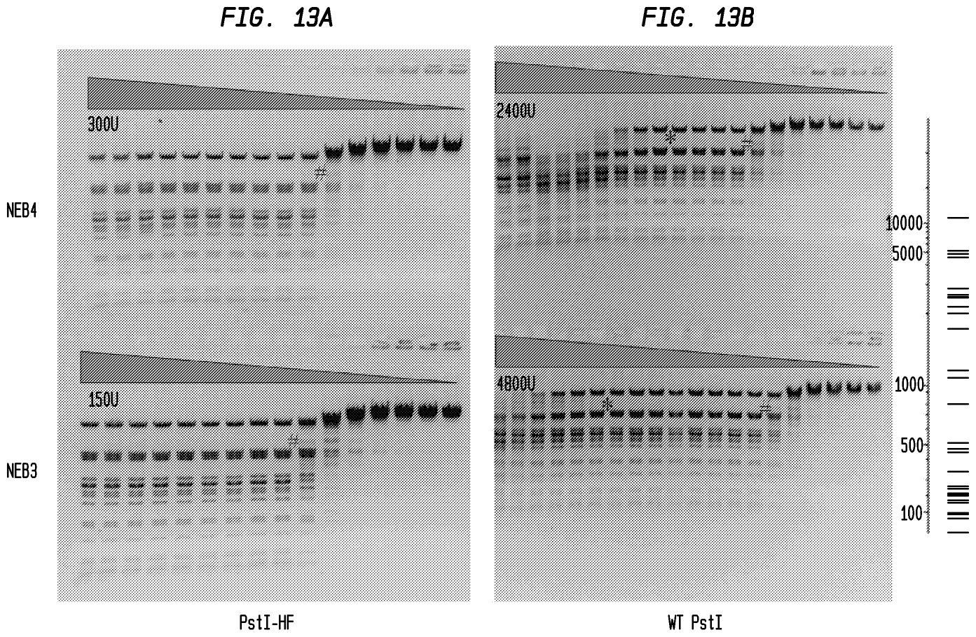

The FI of PstI-HF and WT PstI were measured separately on lambda DNA substrate in NEB1-4 buffers. The dilution buffer is NEB dilution buffer C. The comparison is shown as in the FIG. 13, and the result is listed in Table 12 (below).

TABLE 12
Comparison of PstI-HF and WT PstI

| | PstI-HF | | WT PstI | | |
|---|---|---|---|---|---|
| | | Fidelity | | Fidelity | Improvement |
| Buffer | Activity | Index | Activity | Index | Factor |
| NEB1 | 12.5% | ≥250 | 50% | 32 | ≥8 |
| NEB2 | 100% | ≥2000 | 25% | 16 | ≥125 |
| NEB3 | 25% | ≥500 | 100% | 120 | ≥2 |
| NEB4 | 100% | ≥2000 | 50% | 8 | ≥250 |

PstI-HIF performed best in NEB2 and NEB4, in which the preferred FI is ≥2000; WT PstI performed best in NEB3, in which the FI was 120. The overall FI improvement factor was ≥2000/120=16 times.

### Example 7: Engineering of High Fidelity NcoI

### 1. Expression of NcoI

Expression of NcoI was achieved in E. coli (pSYX20-NcoIM, pRRS-NcoIR). pRRS is a pUC19 derivative plasmid, and pSYX20 is a compatible low copy number plasmid with pRRS vector. The cells were grown at 30° C. overnight in the LB with Amp and Kanamycin (Kan).

### 2. Mutagenesis of NcoI

All 66 charged residues in NcoI were mutated to Ala. These residues were: 7, 8, 19, 22, 27, 30, 31, 32, 33, 37, 39, 42, 46, 55, 56, 61, 62, 64, 68, 69, 75, 84, 88, 89, 92, 93, 95, 97, 100, 116, 136, 144, 146, 162, 166, 170, 178, 183, 185, 187, 188, 189, 196, 199, 202, 204, 209, 211, 212, 213, 216, 219, 227, 229, 237, 241, 244, 250, 251, 257, 259, 261, 268, 279, 282, 285.

The numbers above correspond to amino acid positions in the NcoI protein sequence (SEQ ID NO:88).

The methods were the same as in the previous examples using inverse PCR followed by DpnI digestion. The treated product was then transformed into E. coli (pSYX20-NcoIM).

### 3. Selection of NcoI-HF

The selection of NcoI-HF was similar to that of PstI-HF. The activity was assayed as described above using lambda DNA as substrate with 5% glycerol. Star activity was determined using pBR322 or lambda in 19% DMSO. The following mutations were found to improve star activity: A2T/R31A, D56A, H143A, E166A, R212A and D268A. Among these mutants, NcoI(A2T/R31A) was selected as the NcoI-HF.

### 4. Comparison of NcoI-HF and WT NcoI

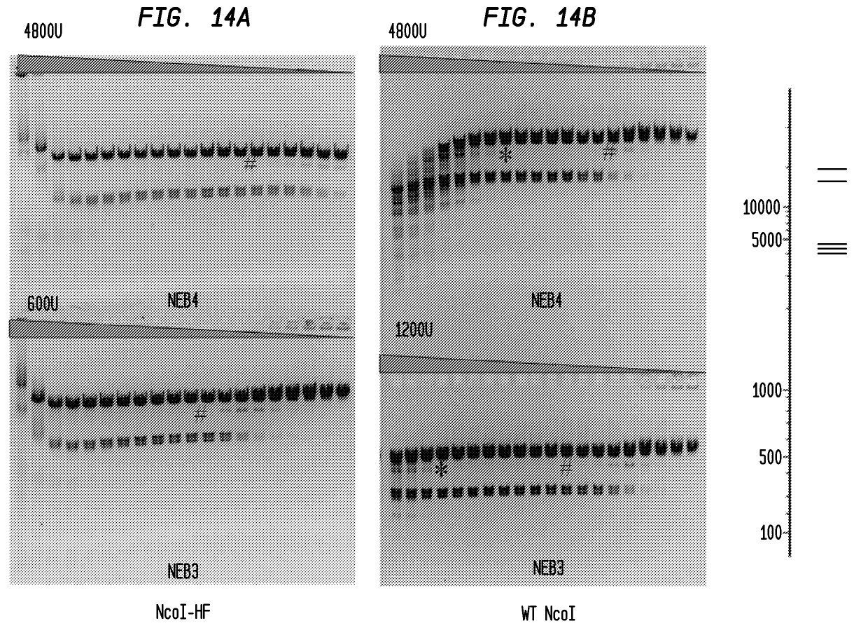

The FIs of NcoI-HF and WT NcoI were determined separately on lambda DNA in NEB1-4 buffers. The comparison is shown in FIG. 14, and the results listed in Table 13 (below).

TABLE 13
Comparison of NcoI-HF with WT NcoI

| | NcoI-HF | | WT NcoI | | |
|---|---|---|---|---|---|
| | | Fidelity | | Fidelity | Improvement |
| Buffer | Activity | Index | Activity | Index | Factor |
| NEB1 | 25% | ≥4000 | 100% | 120 | ≥32 |
| NEB2 | 25% | ≥4000 | 100% | 32 | ≥125 |
| NEB3 | 6.3% | ≥1000 | 25% | 120 | ≥8 |
| NEB4 | 100% | ≥16000 | 100% | 32 | ≥500 |

NcoI-HIF showed the greatest reduction in star activity in NEB4, in which the preferred FI was ≥16000; WT NcoI performed best in NEB1, NEB2 and NEB4, in which the preferred FI was 120. The overall FI improvement factor was ≥16000/120=125.

### Example 8: Engineering of High Fidelity NheI

### 1. Expression of NheI

NheI was expressed in E. coli transformed with pACYC-NheIM, and placzz1-NheIR. placzz1 is a pUC19 derivative plasmid. The cell was grown at 30° C. for overnight in the LB with Amp and Cam.

### 2. Mutagenesis of NheI

All 92 charged residues in NheI were mutated to Ala as the following residues: 5, 6, 7, 14, 17, 19, 22, 25, 28, 31, 38, 39, 42, 47, 49, 52, 56, 58, 59, 60, 64, 74, 75, 76, 77, 80, 91, 93, 104, 105, 110, 112, 116, 117, 123, 126, 130, 131, 133, 135, 137, 147, 149, 152, 159, 160, 165, 167, 170, 171, 174, 179, 183, 195, 202, 205, 207, 209, 210, 211, 214, 216, 218, 221, 225, 231, 241, 243, 244, 250, 252, 256, 257, 259, 264, 266, 267, 281, 285, 287, 288, 289, 291, 297, 300, 307, 313, 315, 318, 321, 324, 325.

The numbers above correspond to amino acid positions in the NheI protein sequence (SEQ ID NO:89).

The methods were the same as in the previous examples using inverse PCR followed by DpnI digestion. The treated product was then transformed into E. coli (pACYC-NheIM).

### 3. Selection of NheI-HF

Selection of NheI-HF was performed according to the previous examples. The standard and star activity assays contained pBR322 as a substrate in NEB4 buffer and 5% glycerol and 39% glycerol, respectively. Only one mutation was found to be significant in improving the NheI. This was E77A. NheI(E77A) was selected as the NheI-HIF.

### 4. Comparison of NheI-HF and WT NheI

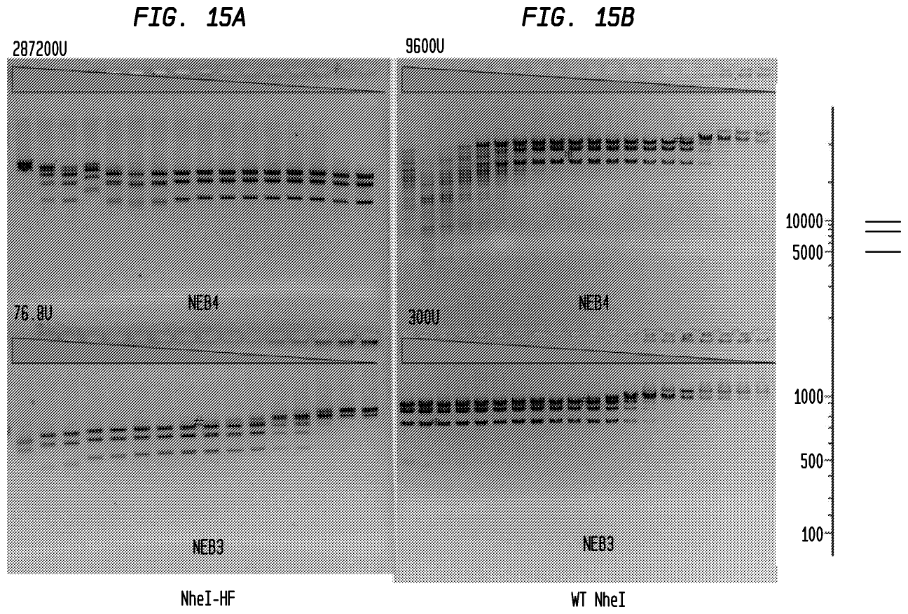

The FIs of NheI-HF and WT NheI were determined separately on pXba, a plasmid substrate containing the XbaI digested piece from Adeno virus in each of NEB1-4 buffers. The comparison is shown in FIG. 15, and the result is listed in Table 14 (below).

TABLE 14
Comparison of NheI-HF and WT NheI

| | NheI-HF | | WT NheI | | |
|---|---|---|---|---|---|
| | | Fidelity | | Fidelity | Improvement |
| Buffer | Activity | Index | Activity | Index | Factor |
| NEB1 | 100% | ≥128000 | 100% | 32 | ≥4000 |
| NEB2 | 3% | ≥4000 | 25% | 120 | ≥32 |
| NEB3 | 0.05% | ≥32 | 12.5% | 120 | ≥0.25 |
| NEB4 | 50% | ≥32000 | 100% | 32 | ≥1000 |

NheI-IF showed optimal activity in NEB1 buffer where its FI is ≥128,000. WT NheI has maximum activity in NEB1 and NEB4 buffers, where its best FI is 32. so, the overall FI improvement factor is ≥128,000/32=≥4000.

### Example 9: Engineering of High Fidelity SspI

### 1. Expression of SspI

SspI was expressed from E. coli transformed with pACYC-SspIM, and placzz1-SspIR. placzz1 is a pUC19 derivative plasmid. The cells were grown at 30° C. overnight in LB with Amp and Cam.

### 2. Mutagenesis of SspI

All 81 charged residues in SspI were mutated to Ala: These were: 3, 8, 12, 13, 18, 19, 20, 35, 40, 42, 44, 47, 52, 60, 62, 65, 68, 69, 72, 74, 76, 77, 78, 79, 83, 85, 88, 89, 90, 96, 100, 108, 109, 118, 119, 127, 128, 129, 131, 132, 137, 144, 153, 154, 155, 156, 158, 165, 168, 170, 172, 177, 178, 179, 181, 185, 186, 187, 191, 194, 195, 197, 202, 204, 215, 222, 229, 237, 240, 246, 250, 256, 257, 259, 260, 264, 265, 267, 268, 269, 274.

The numbers above correspond to amino acid positions in the SspI protein sequence (SEQ ID NO:99).

The methods were the same as in the previous examples using inverse PCR followed by DpnI digestion. The treated product was then transformed into E. coli (pACYC-SspIM).

### 3. Selection of SspI High Fidelity Mutants

The standard cognate and star activity assays of NheI were performed using ΦX174 substrate in NEB 4 buffer and 5% glycerol and 39% glycerol respectively. Mutants #16(H65A), #20(K74A), #23(E78A), #26(E85A), #28(E89A), #33(K109A), #34(E118A), #52(R177A), #62(K197A), #67(D229A) all showed reduced star activity. K109A showed the greatest reduction in star activity. It was decided to seek further improvements in star activity.

4. Further mutations

All residues originally identified as Tyr were mutated to Phe, while other residues Cys, Phe, Met, Asn, Gln, Ser, Thr, and Trp were mutated to Ala. This group included 95 residue mutations at the following positions: 2, 6, 7, 9, 10, 13, 22, 25, 26, 27, 29, 30, 32, 33, 34, 39, 41, 51, 53, 55, 56, 57, 58, 59, 61, 63, 71, 75, 81, 84, 87, 91, 94, 98, 104, 106, 107, 110, 111, 113, 114, 123, 125, 134, 136, 139, 140, 141, 142, 143, 146, 152, 157, 159, 160, 164, 173, 175, 180, 183, 190, 192, 193, 196, 198, 199, 201, 205, 207, 211, 214, 218, 219, 220, 221, 223, 225, 226, 227, 228, 230, 232, 233, 235, 238, 239, 241, 249, 254, 255, 272, 275, 276, 277, 280.

The numbers above correspond to amino acid positions in the SspI protein sequence (SEQ ID NO:113).

The PCRs and the selections were done by the same procedure as above. Among these mutants, it was found that Y98F had least star activity, and it was better than SspI(K109A) in this respect. The SspI(Y98F) was labelled SspI-HF and was deposited as the production strain.

### 5. Comparison of SspI-HF and WT SspI

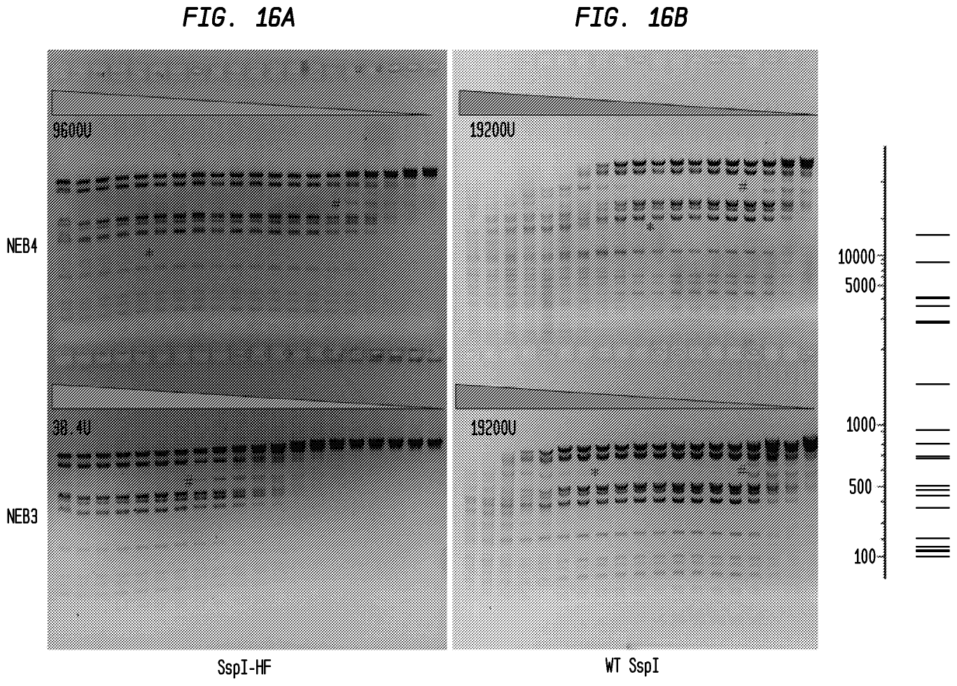

The FIs of SspI-HF and WT SspI were determined separately using lambda DNA substrate in NEB1-4 buffers. The diluent was NEBC. The comparison is shown in FIG. 16, and the result is listed in Table 15 (below).

TABLE 15
Comparison of SspI-HF and WT SspI

| | SspI-HF | | WT SspI | | |
|---|---|---|---|---|---|
| | | Fidelity | | Fidelity | Improvement |
| Buffer | Activity | Index | Activity | Index | Factor |
| NEB1 | 50% | ≥4000 | 100% | 64 | ≥64 |
| NEB2 | 50% | 120 | 100% | 16 | ≥8 |
| NEB3 | 0.6% | ≥32 | 25% | 32 | ≥1 |
| NEB4 | 100% | 500 | 100% | 16 | ≥32 |

SspI-IF performed best in NEB4, in which the preferred FI was 500; WT SspI performed best in NEB1, NEB2 and NEB4, in which the preferred FI was 64. The overall FI improvement factor was 500/64=8.

### Example 10: Engineering of High Fidelity NotI

### 1. Expression of NotI

NotI has significant star activity in NEB4 buffer and less in NEB3 buffer. NotI was engineered to reduce star activity in any NEB buffer. NotI was expressed in competent E. coli transformed with pACYC184-EagIM and placzz2-NotIR. The cells were grown at 37° C. for overnight in the LB with Amp and Cam.

### 2. Mutagenesis of NotI

All 97 charged residues in NotI were mutated to Ala as the following residues: 2, 4, 8, 10, 17, 21, 22, 26, 31, 34, 35, 36, 49, 52, 57, 59, 62, 72, 74, 75, 77, 84, 87, 96, 97, 105, 117, 121, 122, 125, 126, 129, 130, 133, 140, 141, 145, 150, 152, 156, 160, 165, 167, 174, 176, 177, 182, 187, 189, 193, 194, 200, 205, 208, 210, 219, 224, 225, 227, 236, 237, 245, 251, 253, 267, 271, 272, 280, 283, 290, 292, 294, 296, 304, 306, 308, 310, 314, 319, 321, 323, 327, 331, 335, 336, 339, 353, 354, 356, 358, 361, 365, 367, 368, 369, 370, 378, 382.

The numbers above correspond to amino acid positions in the NotI protein sequence (SEQ ID NO:90).

The method for introducing mutants into the enzyme was the same as in the previous examples using inverse PCR followed by DpnI digestion. The treated product was then transformed into E. coli containing pACYC-EagIM.

### 3. Selection of NotI-HF

Selection of NotI-HF was performed as described in the previous examples. The standard cognate and star activity assays used pXba substrate in NEB 4 buffer and 5% glycerol and NEB ExoI buffer (67 mM Glycine-KOH, pH 9.5, 6.7 mM MgCl₂, 10 mM 2-mercaptoethanol) and 37% glycerol respectively. #37(K150A), #44(K176A), #45(R177A), #63(R253A) all showed reduced star activity. K150A was the preferred mutation to reduce star activity. NotI(K150A) was selected as the NotI-HIF.

### 4. Comparison of NotI-HF and WT NotI

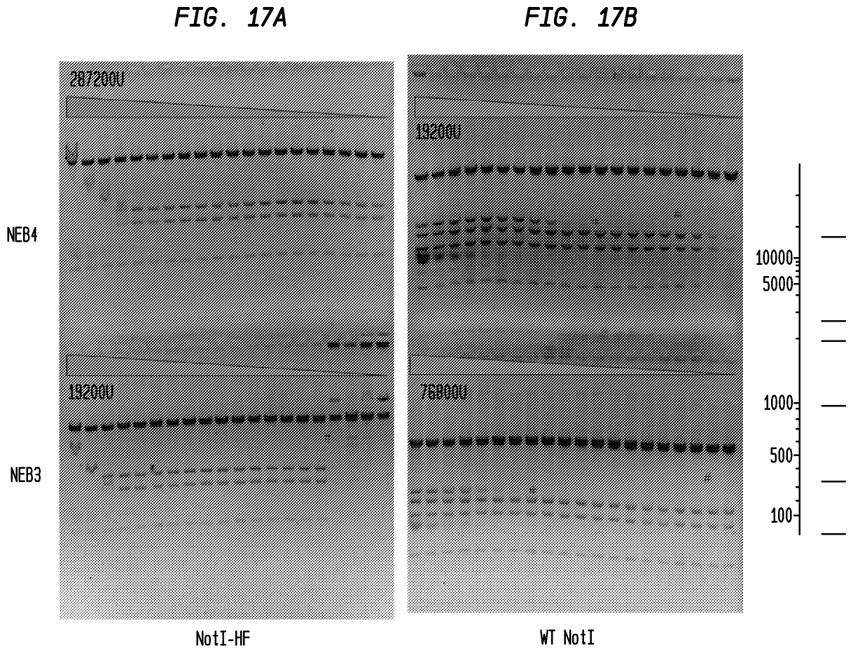

The FIs of NotI-IF and WT NotI were determined separately using pXba substrate in NEB1-4 buffers. The comparison is shown in FIG. 17, and the results are listed in Table 16 (below).

TABLE 16
Comparison of NotI-HF and WT NotI

| | NotI-HF | | WT NotI | | |
|---|---|---|---|---|---|
| | | Fidelity | | Fidelity | Improvement |
| Buffer | Activity | Index | Activity | Index | Factor |
| NEB1 | 6% | ≥8000 | 6% | ≥8000 | ND |
| NEB2 | 100% | ≥128000 | 50% | 250 | ≥512 |
| NEB3 | 1.6% | ≥4000 | 100% | 4000 | ≥1 |
| NEB4 | 50% | ≥64000 | 12.5% | 32 | ≥2000 |

ND: Not determinable, for that both FI is an uncertain number over limit.

NotI-HF performed best in NEB2, in which the preferred FI was ≥128000; WT NheI performed best in NEB3, in which the preferred FI was 4000. The overall fidelity index improvement factor was ≥128000/4000=≥32. Engineering NotI not only further improved the FI of NotI, but also changed the optimal buffer.

### Example 11: Engineering of High Fidelity SacI

### 1. Expression of SacI

SacI was expressed in E. coli transformed with pLG-SacIM and pRRS-SacIR. pRRS is a pUC19 derivative plasmid, pLG is a low copy compatible plasmid. The cells were grown at 30° C. overnight in LB with Amp and Kan.

### 2. Mutagenesis of SacI

All 101 charged residues in SacI were mutated to Ala as the following residues: 6, 7, 11, 15, 16, 19, 24, 25, 29, 30, 39, 40, 42, 45, 58, 61, 62, 63, 65, 67, 70, 71, 72, 74, 75, 76, 81, 85, 94, 98, 104, 105, 114, 116, 120, 123, 127, 129, 133, 134, 141, 143, 144, 145, 146, 150, 151, 154, 169, 170, 172, 181, 187, 196, 197, 200, 201, 211, 216, 220, 221, 224, 227, 228, 232, 238, 240, 246, 248, 250, 258, 270, 271, 277, 281, 288, 289, 295, 296, 297, 299, 303, 306, 313, 314, 321, 322, 324, 332, 336, 337, 340, 342, 344, 345, 347, 349, 350, 353, 357.

The numbers above correspond to amino acid positions in the SacI protein sequence (SEQ ID NO:93).

The methods were the same as in the previous examples using inverse PCR followed by DpnI digestion. The treated product was then transformed into E. coli (pLG-SacIM).

### 3. Selection of SacI-HF

Selection of SacI-HF was achieved using a method that was similar to the previous examples. The standard activity check used pUC19 with 5% glycerol in NEB4 and the star activity check was on pUC19 in NEB4 buffer with 39% glycerol. #52 SacI (Q117H/R154A/L284P) and #60 SacI (Q117H/R200A) both had reduced star activity, and SacI QI 17H/R200A proved to be the preferred mutation. The Q117H was a carry over mutation from the template, which did not affect the activity of SacI. SacI(Q1 17H/R200A) was selected as the SacI-HF.

### 4. Comparison of SacI-HF and WT SacI

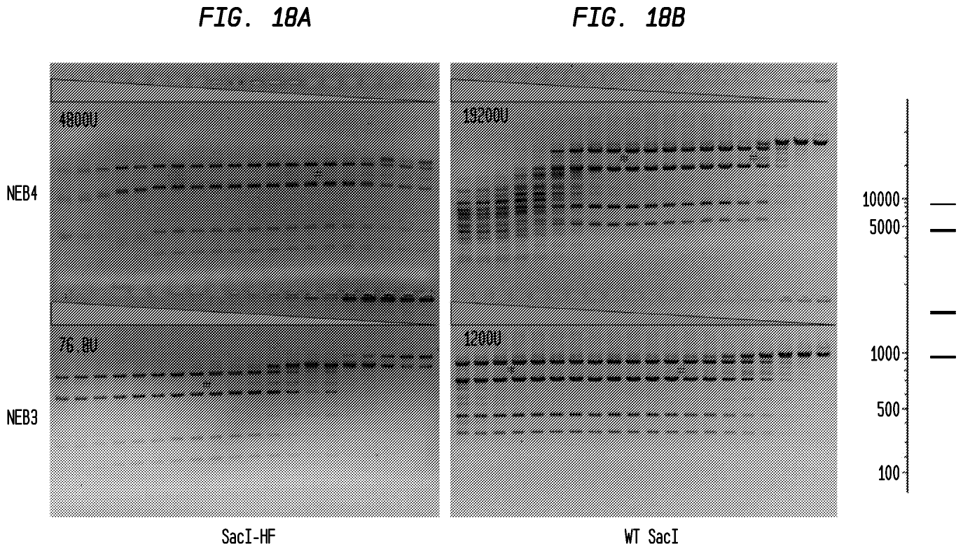

The FIs of SacI-HF and WT SacI were determined separately on pXba substrate in NEB1-4 buffers. The comparison is shown in FIG. 18, and the result is listed in Table 17 (below).

TABLE 17
Comparison of SacI-HF and WT SacI

| | SacI-HF | | WT SacI | | |
|---|---|---|---|---|---|
| | | Fidelity | | Fidelity | Improvement |
| Buffer | Activity | Index | Activity | Index | Factor |
| NEB1 | 25% | ≥2000 | 100% | 120 | ≥16 |
| NEB2 | 12.5% | ≥120 | 50% | 120 | ≥1 |
| NEB3 | 0.8% | ≥120 | 3% | 120 | ≥1 |
| NEB4 | 100% | 4000 | 100% | 32 | 120 |

SacI-HF performed best in NEB4, in which the FI was 4000; WT SacI performed best in NEB1 and NEB4, in which the preferred FI was 120. The overall FI improvement factor was 4000/120=32.

### Example 12: Engineering of High Fidelity PvuII

### 1. Expression of PvuII

PvuII was expressed in E. coli transformed with pACYC-PvuIIM and placzz2-PvuIIR. Placzz2 is a pUC19 derivative plasmid; pACYC is a low copy compatible plasmid.

The cells were grown at 30° C. overnight in LB with Amp and Cam.

### 2. Mutagenesis of PvuII

All 47 charged residues in PvuII were mutated to Ala as the following residues: 3, 5, 8, 11, 15, 18, 21, 25, 26, 30, 34, 38, 54, 55, 58, 61, 66, 68, 70, 75, 78, 83, 84, 85, 93, 95, 105, 110, 114, 116, 118, 119, 121, 125, 126, 128, 129, 130, 134, 136, 137, 138, 143, 147, 151, 152, and 155.

The numbers above correspond to amino acid positions in the PvuII protein sequence (SEQ ID NO:92).

The methods were the same as in the previous examples using inverse PCR followed by DpnI digestion. The treated product was then transformed into E. coli (pACYC-PvuIIM).

### 3. Selection of PvuII High Fidelity Mutants

Selection of PvuII-HF was similar to the previous examples. The standard activity check used lambda DNA substrate with 5% glycerol in NEB4 and the star activity check was on pBR322 in NEB4 buffer with 39% glycerol. None of the mutants were qualified as high fidelity PvuII.

### 4. Additional Mutagenesis Steps

An additional mutagenesis step was mutation of all of the Ser, Thr into Ala, and Tyr to Phe in PvuII. The mutated positions were: 2, 19, 46, 49, 67, 71, 77, 81, 82, 94, 104, 113, 123, 124, 132, 133, 148, 154 and 157.

The methods were the same as in the previous examples using inverse PCR followed by DpnI digestion. The treated product was then transformed into E. coli (pACYC-PvuIIM).

The PvuII(T46A) apped to have less star activity than the WT PvuII, however, further improvement was desired.

T46 was mutated to all other amino acid residues, by changing the codons to the corresponding amino acids. Among all these mutations, T46H, T46K, T46Y, T46G were all better than T46A. T46G is selected as the PvuII-HF.

### 5. Comparison of PvuII-HF and WT PvuII

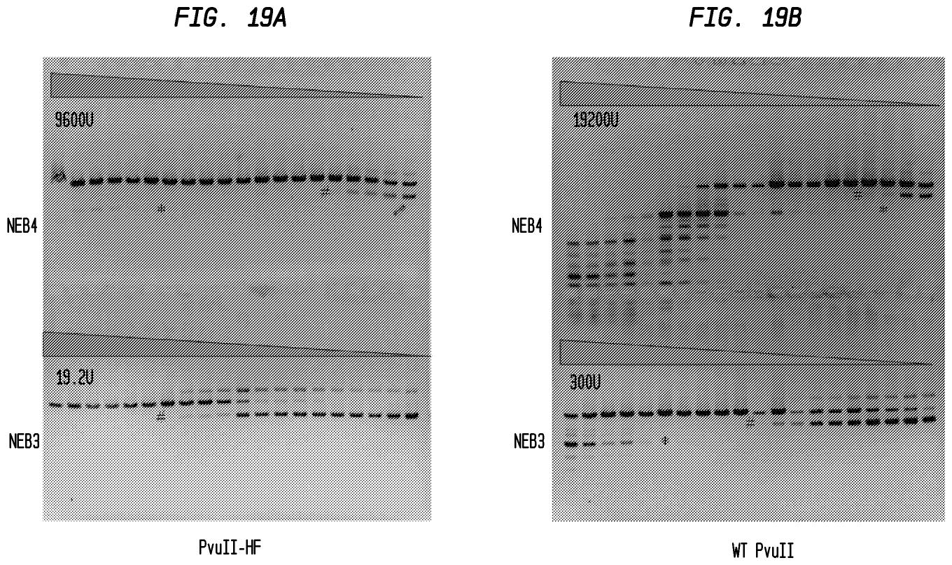

The FIs of PvuII-HF and WT PvuII were determined separately on pBR322, with diluent A in NEB1-4 buffers. The comparison is shown in FIG. 19, and the result is listed in Table 18 (below).

TABLE 18
Comparison of PvuII-HF and WT PvuII

| | PvuII-HF | | WT PvuII | | |
|---|---|---|---|---|---|
| | | Fidelity | | Fidelity | Improvement |
| Buffer | Activity | Index | Activity | Index | Factor |
| NEB1 | 3.1% | ≥250 | 100% | 250 | ≥1 |
| NEB2 | 12.5% | ≥1000 | 25% | 16 | ≥64 |
| NEB3 | 0.4% | ≥32 | 3.1% | 8 | ≥4 |
| NEB4 | 100% | 500 | 100% | ¼ | 2000 |

PvuII-HIF performed best in NEB4, in which the FI was 500; WT PvuII performed best in NEB1 and NEB4, in which the preferred FI was 250. The overall FI improvement factor was 500/250=2. Though the overall FI improvement factor is not high for PvuII, the FI improved 2000 times in NEB4.

### Example 13: Engineering of High Fidelity MfeI

### 1. Expression of MfeI

MfeI was expressed in E. coli transformed with pACYC-MluCIM and pRRS-MfeIR. pRRS is a pUC19 derivative plasmid, pACYC is a low copy compatible plasmid. MluCIM methylate AATT, which is the inner four nucleic acid sequence of the MfeI. The cells were grown at 37° C. overnight in LB with Amp and Cam.

### 2. Mutagenesis of MfeI

The mutagenesis of MfeI was done in three batches. The first batch is all of the charged residues, mutated into Ala as the following amino acid positions: 3, 5, 9, 19, 24, 36, 39, 44, 45, 47, 48, 50, 60, 61, 64, 65, 72, 83, 87, 90, 92, 93, 98, 100, 101, 103, 107, 109, 110, 115, 119, 120, 121, 124, 132, 135, 142, 143, 144, 153, 155, 158, 159, 161, 162, 164, 165, 171, 172, 175, 181, 184, 187, 188, 192, 195, 196, 198, 199, 200; The second batch is all of the residues with hydroxyl group: Ser, Thr and Tyr, with Ser and Thr changed into Ala and Tyr changed into Phe. The residues are at: 4, 7, 21, 28, 38, 40, 43, 53, 74, 75, 76, 81, 89, 91, 112, 122, 127, 134, 136, 157, 167, 170, 173, 177, 185, and 200. The third batch is the residues of Cys, Phe, Met, Asn, Gln, Trp all changed into Ala, the residues are at: 10, 12, 13, 25, 26, 29, 31, 32, 35, 51, 55, 67, 68, 77, 78, 84, 88, 96, 102, 105, 117, 123, 126, 141, 148, 149, 152, 168, 169, 174, 176, 178, 179, 180, 183, 191, 193, 194.

The numbers above correspond to amino acid positions in the MfeI protein sequence (SEQ ID NO:5).

The methods were the same as in the previous examples using inverse PCR followed by DpnI digestion. The treated product was then transformed into E. coli (pACYC-MluCIM).

### 3. Selection of MfeI-HF

Selection of MfeI-HF was achieved using a method that was similar to the previous examples. Cleavage activity was determined using ΦX174 substrate with 5% glycerol in NEB4 and star activity was determined using ΦX174 substrate in NEB4 buffer with 39% glycerol. A significant difficulty for this enzyme was that many mutations improved cleavage activity of the enzyme with reduced star activity, but required higher glycerol concentrations than the WT enzyme. MfeI(K50A) is one example, having reduced star activity and high cleavage activity in high concentration glycerol, while in lower glycerol concentrations, the activity was low. MfeI(Y173A) also reduced star activity. The preferred mutation was Q13A/F35Y. The mutation of F35Y was from the template, and Q13A was a targeted mutation. MfeI(Q13A/F35Y) was labelled MfeI-HF.

### 4. Comparison of MfeI-HF and WT MfeI

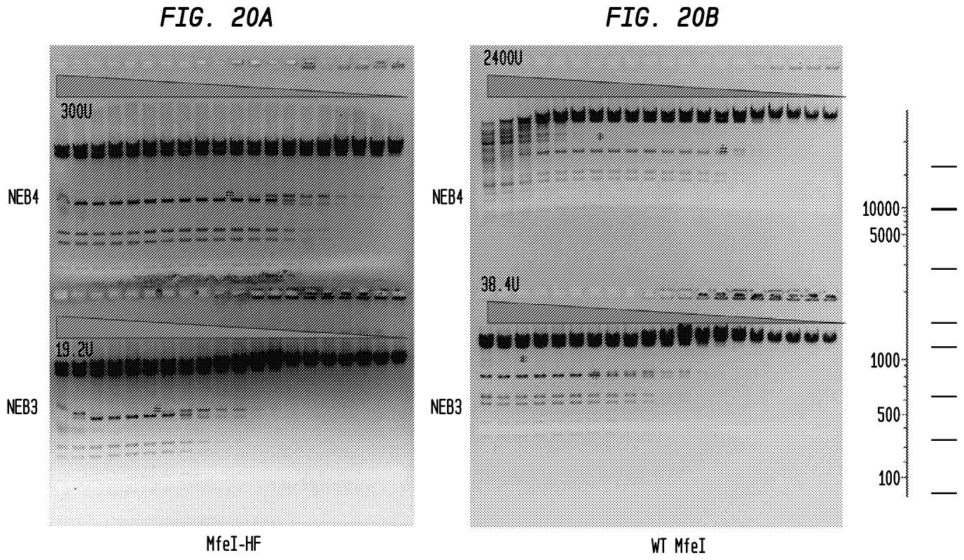

The FIs of MfeI-HF and WT MfeI were determined separately on lambda DNA substrate, with the dilution in NEB diluent A in NEB1-4 buffers. The comparison is shown in FIG. 20, and the result is listed in Table 19 (below).

TABLE 19
Comparison of MfeI-HF and WT MfeI

| | MfeI-HF | | WT MfeI | | |
|---|---|---|---|---|---|
| | | Fidelity | | Fidelity | Improvement |
| Buffer | Activity | Index | Activity | Index | Factor |
| NEB1 | 100% | ≥1000 | 100% | 32 | ≥32 |
| NEB2 | 25% | ≥250 | 12.5% | 16 | ≥16 |
| NEB3 | 1.3% | ≥16 | 6.3% | 8 | ≥2 |
| NEB4 | 100% | ≥500 | 100% | 32 | ≥16 |

MfeI-IF performed best in NEB1 and NEB4, in which the preferred FI was ≥1000; WT MfeI performed best in NEB1 and NEB4, in which the preferred FI was 32. The overall FI improvement factor was ≥1000/32=32 fold.

### Example 14: Engineering of High Fidelity HindIII

### 1. Expression of HindIII

HindIII was expressed in E. coli transformed with pUC19-HindIIIRM, which contains both HindIII endonuclease and methylase genes. The cells were grown at 30° C. overnight in LB with Amp.

### 2. Mutagenesis of HindIII

88 charged residues in HindIII were mutated to Ala. These were: 2, 3, 7, 8, 14, 20, 22, 34, 37, 39, 42, 45, 52, 55, 61, 62, 66, 69, 74, 84, 87, 89, 94, 100, 101, 109, 111, 114, 117, 120, 123, 124, 126, 128, 132, 134, 135, 136, 137, 138, 153, 158, 162, 163, 171, 172, 180, 182, 183, 190, 197, 198, 201, 202, 207, 209, 214, 215, 218, 222, 225, 227, 228, 229, 237, 238, 243, 244, 245, 249, 250, 251, 254, 255, 261, 265, 266, 267, 270, 274, 275, 281, 283, 286, 290, 293, 296, 297.

All residues Cys, Met, Asn, Gln, Ser, Thr, Trp were changed to Ala while Tyr was changed to Phe at the positions of 4, 11, 15, 17, 18, 19, 21, 23, 26, 27, 30, 31, 36, 38, 46, 57, 58, 59, 60, 63, 64, 76, 77, 80, 82, 83, 88, 91, 99, 102, 103, 104, 112, 113, 116, 118, 121, 122, 125, 131, 133, 139, 143, 146, 147, 148, 149, 151, 152, 154, 155, 157, 159, 160, 164, 168, 169, 170, 178, 184, 185, 187, 188, 189, 191, 193, 194, 195, 199, 200, 203, 204, 206, 210, 211, 212, 213, 216, 217, 219, 220, 221, 224, 230, 232, 233, 236, 240, 241, 246, 252, 253, 256, 258, 262, 263, 264, 277, 278, 279, 280, 284, 287, 288, 294, 295, 299.

The numbers above correspond to amino acid positions in the HindIII protein sequence (SEQ ID NO:85).

The methods were the same as in the previous examples using inverse PCR followed by DpnI digestion. The treated product was then transformed into E. coli strain ER3081.

### 3. Selection of HindIII-HF

Selection of HindIII-HF was achieved using a method that was similar to the previous examples. The standard activity check used lambda DNA with 5% glycerol in NEB4 and star activity was measured using lambda DNA substrate in NEB4 buffer with 39% glycerol. 2 mutants of HindIII were found to have reduced star activity. These were HindIII(K198A) and S188P/E190A. HindIII(K198A) was labelled HindIII-HF.

### 4. Comparison of HindIII-HF and WT HindIII

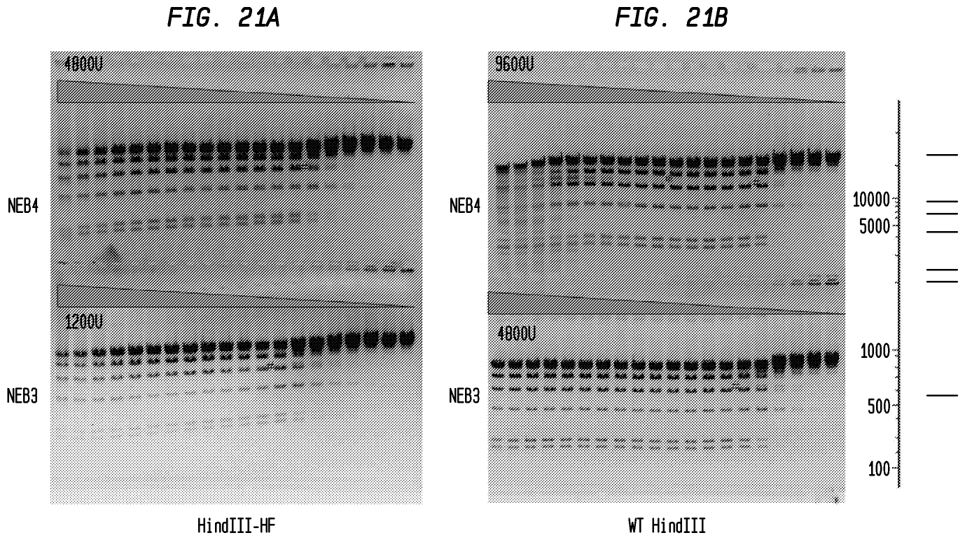

The FIs of HindIII-HF and WT HindIII were determined separately using lambda DNA substrate in each of NEB1-4 buffers with diluent B. The comparison is shown in FIG. 21, and the result is listed in Table 20 (below).

TABLE 20
Comparison of HindIII-HF and WT HindIII

| | HindIII-HF | | WT HindIII | | |
|---|---|---|---|---|---|
| | | Fidelity | | Fidelity | Improvement |
| Buffer | Activity | Index | Activity | Index | Factor |
| NEB1 | 25% | ≥16000 | 25% | 32 | ≥500 |
| NEB2 | 100% | ≥64000 | 100% | 250 | ≥250 |
| NEB3 | 12.5% | ≥16000 | 25% | 4000 | ≥4 |
| NEB4 | 50% | ≥32000 | 50% | 32 | ≥1000 |

HindIII-IF performed best in NEB2, in which the preferred FI was ≥64000; WT HindIII performed best in NEB2, in which the preferred FI was 250. The overall FI improvement factor was 4000/120=32.

### Example 15: Engineering of High Fidelity SbfI

### 1. Expression of SbfI

SbfI was expressed in E. coli transformed with pUC19-SbfIRM. The cells were grown at 30° C. overnight in LB with Amp.

### 2. Mutagenesis of SbfI

78 charged residues in SbfI were mutated to Ala. These were: 5, 8, 15, 18, 23, 27, 30, 34, 46, 49, 50, 53, 58, 63, 66, 70, 71, 74, 81, 82, 83, 85, 86, 87, 90, 94, 103, 115, 120, 121, 127, 132, 135, 136, 143, 144, 147, 150, 152, 154, 164, 169, 170, 183, 184, 187, 188, 192, 196, 204, 206, 208, 213, 214, 215, 218, 219, 226, 228, 230, 233, 237, 238, 239, 241, 248, 251, 253, 257, 258, 259, 260, 262, 266, 282, 284, 285, 288, 293, 297, 299, 304, 305, 307, 311, 316, and 322.

The residues of Ser and Thr in SbfI were also mutated into Ala. Tyr was mutated into Phe. The following positions were targeted: 3, 4, 5, 10, 13, 16, 31, 35, 38, 54, 55, 56, 68, 76, 78, 80, 88, 109, 111, 116, 119, 129, 131, 137, 146, 162, 174, 197, 198, 201, 205, 210, 224, 252, 263, 270, 272, 286, 298, 315, 321.

Another 55 residues of Cys, Phe, Met, Asn, Gln, Trp were also mutated to Ala at positions of: 2, 24, 26, 29, 32, 51, 62, 65, 67, 72, 84, 91, 92, 95, 97, 101, 104, 106, 110, 112, 114, 117, 124, 134, 140, 157, 160, 171, 178, 179, 185, 189, 193, 212, 217, 225, 231, 243, 245, 247, 256, 265, 268, 277, 279, 280, 281, 283, 287, 289, 290, 296, 301, 313 and 317.

The numbers above correspond to amino acid positions in the SbfI protein sequence (SEQ ID NO:96).

The methods were the same as in the previous examples using inverse PCR followed by DpnI digestion. The mutated products were transformed into E. coli strain ER2984.

### 3. Selection of SbfI-HF

Selection of SbfI-HF was achieved as described in previous examples. The standard activity check used lambda DNA with 5% glycerol in NEB4 and the star activity check was on lambda DNA in Exonuclease I buffer. SbfI(K251A) was labelled SbfI-HF.

### 4. Comparison of SbfI-HF and WT SbfI

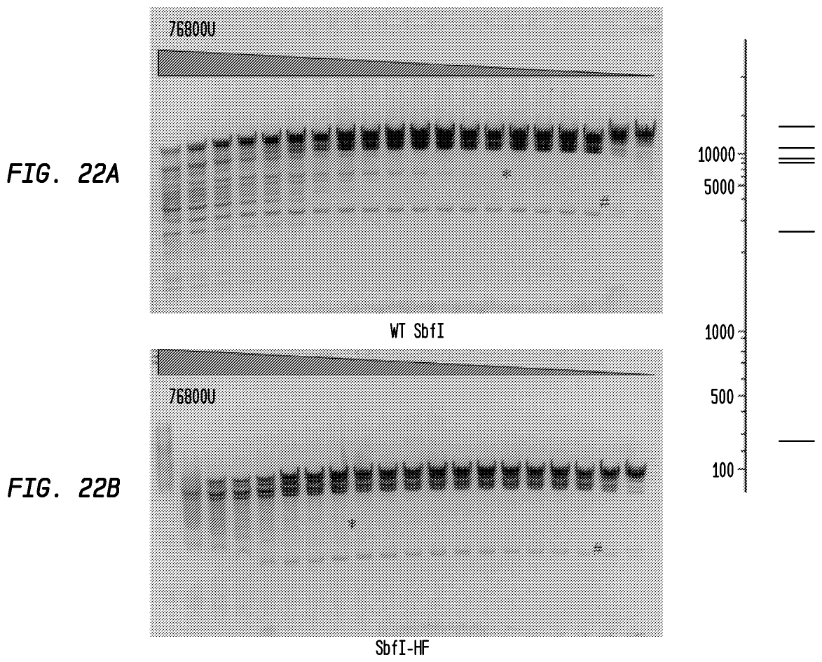

The FIs of SbfI-HF and WT SbfI were determined separately on lambda DNA in NEB1-4 buffers with diluent C. The comparison is shown in FIG. 22, and the result is listed in Table 21 (below).

TABLE 21
Comparison of SbfI-HF and WT SbfI

| | SbfI-HF | | WT SbfI | | |
|---|---|---|---|---|---|
| | | Fidelity | | Fidelity | Improvement |
| Buffer | Activity | Index | Activity | Index | Factor |
| NEB1 | 100% | 1000 | 100% | 16 | 64 |
| NEB2 | 50% | 120 | 50% | 32 | 4 |
| NEB3 | 3.5% | 8 | 25% | 120 | 1/16 |
| NEB4 | 100% | 250 | 12.5% | 8 | 32 |

SbfI-HF performed best in NEB1 and NEB4, in which the preferred FI was 1000; WT SbfI performed best in NEB1, in which the preferred FI was 8. The overall FI improvement factor was 1000/8=125 fold.

### Example 16: Engineering of High Fidelity EagI

### 1. Expression of EagI

EagI was expressed in E. coli transformed with pBR322-EagIRM. The cells were grown at 30° C. overnight in LB with 20 μg/ml Tetracycline.

### 2. Mutagenesis of EagI

Asp, Glu, His, Lys, Arg, Ser, Thr, Asn and Gln residues were mutated to Ala. Tyr was mutated to Phe. These were the following residues: 2, 3, 4, 5, 6, 9, 13, 14, 17, 19, 21, 23, 27, 35, 36, 37, 40, 42, 43, 44, 45, 46, 49, 51, 53, 55, 56, 58, 60, 66, 67, 69, 71, 72, 73, 74, 75, 77, 78, 80, 82, 86, 87, 92, 93, 94, 95, 98, 99, 100, 102, 103, 104, 105, 112, 113, 114, 116, 117, 119, 122, 125, 127, 132, 134, 135, 137, 139, 140, 141, 145, 147, 148, 150, 152, 154, 155, 156, 157, 160, 162, 163, 164, 166, 169, 172, 173, 176, 177, 178, 179, 182, 185, 187, 188, 189, 193, 196, 197, 201, 202, 203, 204, 205, 206, 208, 209, 212, 217, 220, 221, 222, 224, 225, 230, 235, 236, 237, 238, 239, 240, 241, 243, 245, 246, 247, 248, 251, 255, 257, 258, 259, 260, 263, 264, 265, 266, 270, 272, 273, 275, 276, 277, 279, 280, 283, 286, 288, 289, 291, 295, 296.

The numbers above correspond to amino acid positions in the EagI protein sequence (SEQ ID NO:82).

The methods were the same as in the previous examples using inverse PCR followed by DpnI digestion. The treated product was then transformed into E. coli strain ER 3081 and grown on the LB agar plate with Tetracycline.

### 3. Selection of EagI-HF

Selection of EagI-HF was achieved using a method that was different to the previous examples which used high concentration of glycerol, high concentration of DMSO or high pH. Since the expression was too low to show the star activity in the crude extract, it would be very tedious to purify each of the mutants to check the star activity. From the previous examples, it was deduced that HF endonucleases tended to have increased cleavage activity in NEB4 compared to NEB3. Hence, the activity of EagI in the crude extract was measured in both NEB3 and NEB4; the one with highest ratio of NEB4/NEB3 was selected. EagI(H43A) was labelled EagI-HF.

### 4. Comparison of EagI-HF and WT EagI

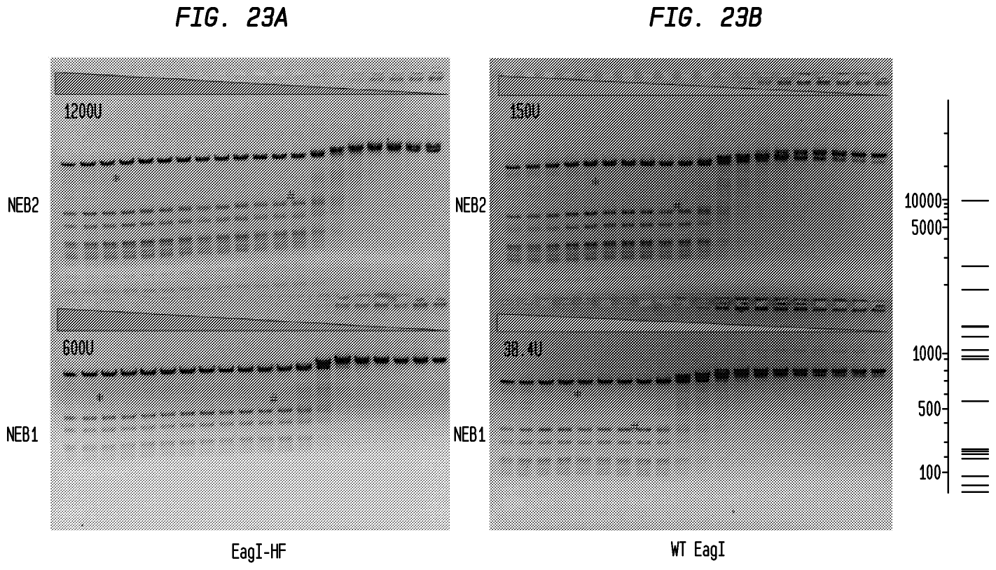

The FIs of EagI-HF and WT EagI were determined separately on pXba substrate in each of NEB1-4 buffers. The comparison is shown in FIG. 23, and the result is listed in Table 22 (below).

TABLE 22
Comparison of EagI-HF and WT EagI

| | EagI-HF | | WT EagI | | |
|---|---|---|---|---|---|
| | | Fidelity | | Fidelity | Improvement |
| Buffer | Activity | Index | Activity | Index | Factor |
| NEB1 | 50% | 250 | 25% | 4 | 64 |
| NEB2 | 100% | 250 | 50% | 8 | 32 |
| NEB3 | 50% | 250 | 100% | 250 | 1 |
| NEB4 | 100% | 500 | 100% | 16 | 16 |

EagI-HF performed best in NEB2 and NEB4, in which the preferred FI was 500; WT EagI performed best in NEB3 and NEB4, in which the preferred FI was 250. The overall FI improvement factor was 500/250=2.

### Example 17: Engineering of High Fidelity EcoRV

### 1. Expression of EcoRV

EcoRV was expressed in E. coli strain transformed with pACYC-EcoRVM and placzz1-EcoRV. Placzz1 is a pUC19 derivative plasmid and pACYC is a low copy compatible plasmid. The cells were grown at 37° C. overnight in LB with Amp and Cam.

### 2. Mutagenesis of EcoRV

Cys, Asp, Glu, Phe, His, Lys, Met, Asn, Gln, Arg, Ser, Thr, and Trp residues were changed to Ala. Tyr was changed to Phe. These were: 2, 4, 5, 6, 9, 12, 13, 14, 15, 16, 17, 18, 19, 21, 25, 27, 29, 31, 35, 36, 37, 38, 41, 42, 44, 45, 47, 48, 49, 53, 54, 57, 58, 59, 61, 64, 65, 67, 68, 69, 70, 71, 72, 74, 75, 76, 78, 79, 81, 82, 84, 85, 86, 90, 92, 93, 94, 95, 96, 97, 98, 99, 100, 101, 102, 104, 105, 106, 110, 111, 112, 113, 114, 115, 116, 117, 118, 119, 120, 123, 125, 126, 127, 128, 131, 132, 136, 138, 139, 140, 143, 144, 145, 146, 147, 149, 150, 151, 152, 154, 155, 157, 158, 161, 163, 164, 167, 169, 171, 172, 173, 174, 179, 183, 185, 186, 187, 188, 191, 193, 195, 196, 197, 198, 199, 201, 203, 206, 207, 208, 209, 210, 211, 212, 214, 215, 216, 217, 218, 219, 220, 221, 222, 223, 224, 226, 227, 228, 229, 230, 231, 232, 234, 235, 236, 237, 238, 239, 241, 242, 244, and 245.

The numbers above correspond to amino acid positions in the EcoRV protein sequence (SEQ ID NO:84).

The methods were the same as in the previous examples using inverse PCR followed by DpnI digestion. The treated product was then transformed into E. coli (pACYC-EcoRVM).

### 3. Selection of EcoRV-HF

Selection of EcoRV-HF was achieved using a method that was similar to the previous examples. The standard activity check used pXba with 5% glycerol in NEB4 and the star activity check was on pXba in Exonuclease I buffer with 39% glycerol.

EcoRV(D19A/E27A) was found to have reduced star activity compared with WT EcoRV. This mutant was labeled EcoRV-HF. For this mutant, the E27A was the targeted mutation, and D19A was a spontaneous mutation. The double mutant had greater reduction in star activity than either the D19A and E27A single mutant.

### 4. Comparison of EcoRV-HF and WT EcoRV

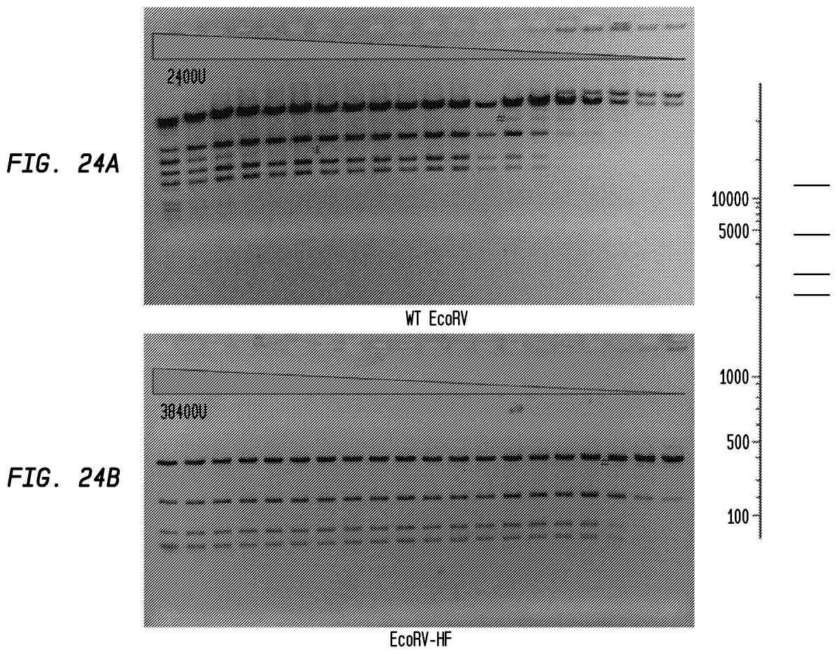

The FIs of EcoRV-HF and WT EcoRV were determined separately on pXba substrate in each of NEB1-4 buffers. The comparison is shown in FIG. 24, and the result is listed in Table 23 (below).

TABLE 23
Comparison of EcoRV-HF and WT EcoRV

| | EcoRV-HF | | WT EcoRV | | |
|---|---|---|---|---|---|
| | | Fidelity | | Fidelity | Improvement |
| Buffer | Activity | Index | Activity | Index | Factor |
| NEB1 | 25% | ≥16000 | 6.3% | 32 | ≥500 |
| NEB2 | 100% | ≥64000 | 50% | 120 | ≥500 |
| NEB3 | 50% | ≥32000 | 100% | 1000 | ≥32 |
| NEB4 | 100% | ≥64000 | 25% | 64 | ≥1000 |

EcoRV-HF performed best in NEB2 and NEB4, in which the preferred FI was ≥64000; WT EcoRV performed best in NEB3, in which the preferred FI was 1000. The overall FI improvement factor was ≥64000/1000=64.

### Example 18: Engineering of High Fidelity AvrII

### 1. Expression of AvrII

AvrII was expressed in E. coli transformed with pUC19-AvrIIRM. The cells were grown at 30° C. overnight in LB with Amp.

### 2. Mutagenesis of AvrII

Cys, Asp, Glu, Phe, His, Lys, Met, Asn, Gln, Arg, Ser, Thr, and Trp residues were mutated to Ala. Tyr was changed to Phe. These were: 2, 3, 4, 6, 8, 9, 10, 12, 15, 17, 19, 20, 22, 23, 27, 29, 30, 31, 32, 34, 36, 40, 41, 42, 43, 44, 46, 47, 48, 50, 51, 53, 55, 56, 57, 58, 59, 60, 65, 68, 70, 72, 74, 75, 76, 77, 79, 80, 82, 83, 84, 86, 87, 88, 94, 95, 96, 97, 100, 104, 105, 106, 107, 108, 110, 112, 113, 116, 117, 119 120, 121, 122, 123, 124, 126, 127, 129, 130, 131, 132, 134, 136, 139, 142, 143, 144, 145, 150, 151, 152, 153, 154, 156, 157, 158, 161, 163, 164, 165, 166, 168, 169, 173, 174, 177, 178, 181, 182, 184, 186, 187, 188, 189, 190, 191, 192, 195, 198, 200, 202, 206, 207, 211, 215, 216, 220, 223, 224, 226, 229, 230, 231, 232, 233, 234, 235, 236, 237, 239, 243, 244, 245, 246, 248, 249, 253, 255, 256, 260, 262, 264, 265, 266, 267, 268, 269, 270, 272, 274, 276, 277, 278, 279, 280, 281, 284, 285, 286, 288, 289, 290, 291, 299, 302, 303, 304, 305, 306, 308, 310, 312, 314, 315, 316, 318, 321, 322, 324, 325, 328, 331, 333, 335, 337, 338, 339, 340, 342, 343, 346, 347, 348, 350, 351, 353, 354, 355, 356, 358.

The numbers above correspond to amino acid positions in the AvrII protein sequence (SEQ ID NO:80).

The methods were the same as in the previous examples using inverse PCR followed by DpnI digestion. The treated product was then transformed into E. coli expression strain ER2984.

### 3. Selection of AvrII-HF

Selection of AvrII-HF was achieved using a method that was similar to the previous examples. The cleavage activity was determined using pBC4 with 5% glycerol in NEB4 and the star activity was measured using pBC4 in ExoI buffer with 39% glycerol. Mutants #16 (M29A), #57(E96A), #60(Y104F), #62(K106A), #154(S127A), #170(F142A) all showed improvement. AvrII(Y104F) was labelled AvrII-HF.

### 4. Comparison of AvrII-HF and WT AvrII

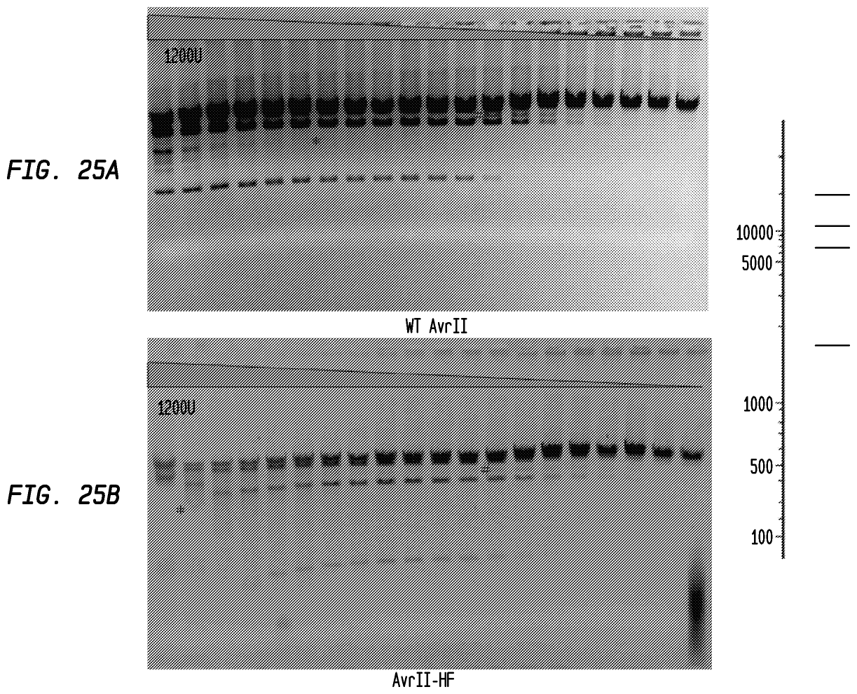

The FIs of AvrII-HF and WT AvrII were determined separately on T7 DNA substrate with diluent B in each of NEB1-4 buffers. The comparison is shown in FIG. 25, and the result is listed in Table 24 (below).

TABLE 24
Comparison of AvrII-HF and WT AvrII

| | AvrII-HF | | WT AvrII | | |
|---|---|---|---|---|---|
| | | Fidelity | | Fidelity | Improvement |
| Buffer | Activity | Index | Activity | Index | factor |
| NEB1 | 100% | 500 | 100% | 64 | ≥8 |
| NEB2 | 50% | ≥500 | 100% | 8 | ≥64 |
| NEB3 | 3.1% | ≥16 | 25% | 32 | ≥0.5 |
| NEB4 | 100% | 1000 | 100% | 32 | 32 |

AvrII-IF performed best in NEB1 and NEB4, in which the preferred FI was 1000; WT AvrII performed best in NEB1 and NEB4, in which the preferred FI was 64. The overall FI improvement factor was 1000/64=16.

### Example 19: Engineering of High Fidelity BstXI

### 1. Expression of BstXI

BstXI was expressed in E. coli transformed with pACYCBstXIMS and pUC19-BstXIR. pACYC is a low copy compatible plasmid. The BstXI has to have both Methylase gene and the specificity gene to have a methylase function. The cells were grown at 37° C. overnight in LB with Amp and Cam.

### 2. Mutagenesis of BstXI

237 amino acid mutations were made in BstXI as follows. Cys, Asp, Glu, Phe, His, Lys, Met, Asn, Gln, Arg, Ser, Thr, Trp were mutated to Ala. Try was mutated to Phe. These were: 4, 6, 7, 9, 11, 12, 14, 15, 17, 18, 20, 21, 22, 23, 24, 26, 27, 29, 30, 31, 32, 33, 34, 35, 36, 37, 39, 40, 42, 43, 46, 48, 50, 53, 54, 57, 58, 59, 60, 62, 63, 64, 65, 66, 71, 72, 73, 75, 76, 78, 80, 81, 82, 83, 84, 86, 89, 91, 93, 94, 95, 96, 97, 98, 103. 105, 106, 108, 110, 111, 112, 114, 117, 118, 120, 123, 124, 125, 126, 127, 128, 129, 130, 131, 137, 138, 139, 141, 142, 144, 145, 146, 148, 151, 152, 153, 154, 155, 156, 159, 162, 163, 166, 168, 169, 171, 172, 173, 174, 175, 176, 177, 178, 182, 185, 188, 189, 191, 193, 194, 195, 196, 198, 199, 201, 204, 208, 209, 210, 211, 212, 214, 215, 216, 217, 218, 219, 220, 221, 223, 228, 229, 230, 233, 235, 236, 238, 239, 240, 244, 245, 248, 249, 250, 253, 254, 255, 258, 259, 260, 261, 263, 264, 265, 267, 268, 269, 272, 276, 277, 278, 279, 280, 282, 285, 286, 287, 288, 289, 291, 293, 294, 295, 300, 301, 302, 304, 305, 306, 308, 309, 312, 314, 317, 318, 319, 320, 323, 324, 325, 326, 330, 331, 333, 334, 335, 337, 343, 344, 345, 346, 347, 349, 353, 355, 356, 357, 358, 359, 360, 362, 363, 364, 365, 367, 369, 371, 373, 374, 376, 377, 378, 379, 380, 381, 382, and 383.

The numbers above correspond to amino acid positions in the BstXI protein sequence (SEQ ID NO:7).

The methods were the same as in the previous examples using inverse PCR followed by DpnI digestion. The treated product was then transformed into E. coli (pACYC-BstXIMS).

### 3. Selection of BstXI-HF

Selection of BstXI-HF was achieved using a method that was similar to the previous examples. The cleavage activity was determined using pBC4 with 5% glycerol in NEB4 and the star activity was determined using pBC4 DNA substrate in NEB4 buffer with 39% glycerol. Mutants #36(Y57F), #44(N65A), #48(E75A), #49(N76A), and #124(K199A) all had reduced star activity. The BstXI(N65A) was labelled BstXI-HF.

### 4. Comparison of BstXI-HF and WT BstXI

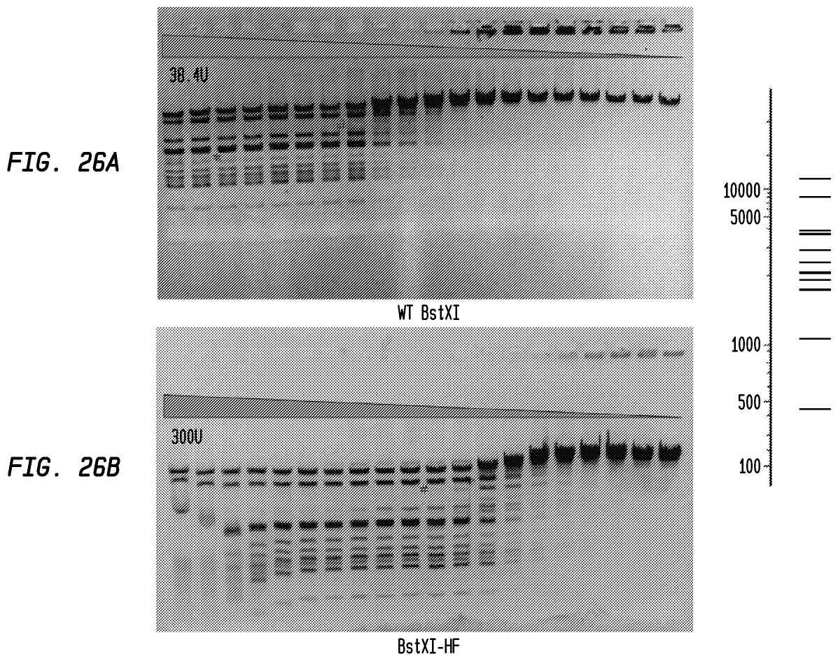

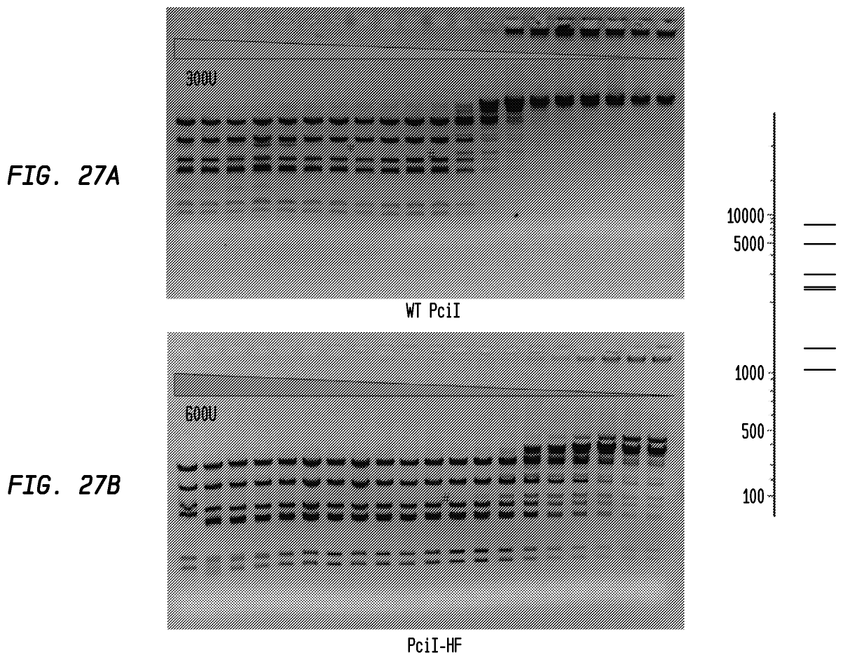

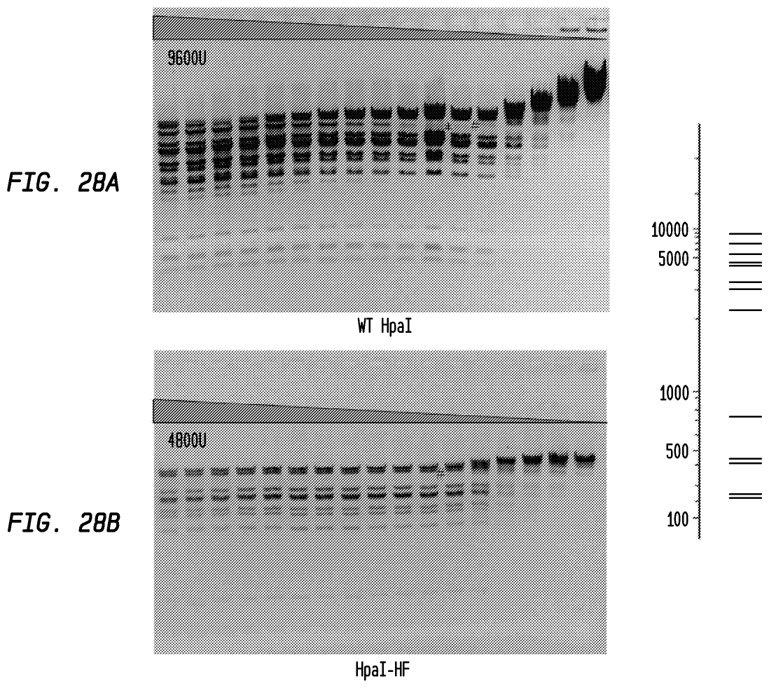

The FIs of BstXI-HF and WT BstXI were determined separately on lambda DNA substrate with diluent A in each of NEB1-4 buffers. The comparison is shown in FIG. 26, and the result is listed in Table 25 (below).

TABLE 25
Comparison of BstXI-HF and WT BstXI

| | BstXI-HF | | WT BstXI | | |
|---|---|---|---|---|---|
| | | Fidelity | | Fidelity | Improvement |
| Buffer | Activity | Index | Activity | Index | Factor |
| NEB1 | 50% | ≥120 | 6.3% | 4 | ≥32 |
| NEB2 | 100% | ≥250 | 100% | 32 | ≥8 |
| NEB3 | 6.3% | ≥16 | 100% | 2 | ≥8 |
| NEB4 | 100% | ≥250 | 100% | 32 | ≥32 |

BstXI-HIF performed best in NEB2 and NEB4, in which the preferred FI was ≥250; WT BstXI performed best in NEB2, NEB3 and NEB4, in which the preferred FI was 32. The overall FI improvement factor was ≥250/32=8.

### Example 20: Engineering of High Fidelity PciI

### 1. Expression of PciI

PciI was expressed in E. coli transformed with pACYC-PciIM and placzz1-PciIR. placzz1 is a pUC19 derivative plasmid, pACYC is a low copy compatible plasmid. The cells were grown at 37° C. overnight in LB with Amp and Cam.

### 2. Mutagenesis of PciI

151 amino acid residues in PciI were mutated with Cys, Asp, Glu, Phe, His, Lys, Met, Asn, Gln, Arg, Ser, Thr. Trp was changed to Ala and Tyr to Phe. These were: 2, 3, 4, 6, 8, 9, 10, 11, 12, 14, 17, 18, 19, 21, 24, 25, 26, 28, 29, 30, 31, 33, 34, 35, 36, 38, 39, 41, 44, 46, 47, 49, 50, 51, 54, 55, 56, 58, 59, 60, 63, 67, 68, 69, 71, 74, 75, 78, 80, 81, 82, 85, 86, 91, 92, 95, 97, 98, 101, 103, 104, 107, 109, 113, 114, 115, 118, 119, 120, 121, 122, 124, 126, 127, 129, 130, 131, 132, 133, 135, 136, 137, 138, 143, 145, 146, 147, 148, 149, 151, 152, 153, 154, 155, 157, 158, 159, 161, 164, 165, 167, 172, 175, 178, 179, 180, 182, 184, 185, 186, 190, 192, 193, 196, 197, 198, 199, 200, 202, 203, 206, 207, 209, 210, 215, 218, 221, 222, 228, 229, 230, 231, 232, 233, 234, 235, 237, 238, 239, 241, 242, 243, 244, 246, 247, 248, 253, 254, 255, 256.

The numbers above correspond to amino acid positions in the PciI protein sequence (SEQ ID NO:15).

The methods were the same as in the previous examples using inverse PCR followed by DpnI digestion. The treated product was then transformed into E. coli (pACYC-PciIM).

### 3. Selection of PciI-HF

Selection of PciI-HF was achieved using a method that was similar to the previous examples. The cleavage activity was determined using SalI-cut pBR322 with 5% glycerol in NEB4 and the star activity was determined using SalI-cut pBR322 in ExoI buffer with 39% glycerol. A double mutant PciI(E78A/S133A) had reduced star activity and strong cleavage activity. This mutant was not one of the targeted mutations described above, but was a fortuitous random event.

### 4. Comparison of PciI-HF and WT PciI

The FIs of PciI-HF and WT PciI were determined separately on pXba substrate with diluent A in each of NEB1-4 buffers. The comparison is shown in FIG. 27, and the result is listed in Table 26 (below).

TABLE 26
Comparison of PciI-HF and WT PciI

| | PciI-HF | | WT PciI | | |
|---|---|---|---|---|---|
| | | Fidelity | | Fidelity | Improvement |
| Buffer | Activity | Index | Activity | Index | Factor |
| NEB1 | NC | NC | 50% | 2000 | N/A |
| NEB2 | 100% | ≥2000 | 25% | 16 | ≥120 |
| NEB3 | 100% | ≥2000 | 100% | 120 | ≥16 |
| NEB4 | 100% | ≥1000 | 12.5% | 8 | ≥120 |

PciI-HF performed best in NEB2, NEB3 and NEB4, in which the preferred FI was ≥2000; WT PciI performed best in NEB3, in which the preferred FI was 120. The overall FI improvement factor was ≥2000/120=16.

### Example 21: Engineering of High Fidelity HpaI

### 1. Expression of HpaI

HpaI was expressed in E. coli transformed with pACYC-MseIM and placzz1-HpaIR. placzz1 is a pUC19 derivative plasmid, pACYC is a low copy compatible plasmid. The cells were grown at 37° C. overnight in LB with Amp and Cam.

### 2. Mutagenesis of Hpa

156 amino acid residues in HpaI were mutated with Cys, Asp, Glu, Phe, His, Lys, Met, Asn, Gln, Arg, Ser, and Thr. Trp was changed to Ala and Tyr to Phe. These were: 7, 8, 9, 13, 14, 16, 17, 19, 20, 21, 22, 23, 26, 27, 29, 30, 33, 34, 35, 36, 37, 38, 40, 41, 42, 46, 47, 48, 50, 51, 56, 57, 59, 60, 65, 67, 69, 71, 72, 74, 75, 78, 79, 80, 81, 82, 83, 84, 85, 86, 89, 91, 93, 94, 95, 99, 100, 104, 105, 106, 108, 109, 110, 113, 115, 117, 119, 121, 122, 123, 124, 127, 128, 130, 131, 133, 135, 136, 137, 138, 139, 141, 142, 146, 147, 149, 150, 152, 156, 158, 159, 160, 162, 164, 165, 166, 167, 168, 169, 170, 172, 173, 176, 177, 180, 181, 182, 184, 185, 187, 188, 190, 191, 192, 193, 195, 196, 197, 202, 204, 206, 208, 209, 211, 212, 214, 215, 216, 217, 218, 219, 220, 221, 222, 223, 224, 225, 228, 230, 231, 233, 234, 235, 236, 237, 238, 240, 241, 242, 243, 244, 245, 247, 248, 249.

The numbers above correspond to amino acid positions in the HpaI protein sequence (SEQ ID NO:86).

The methods were the same as in the previous examples using inverse PCR followed by DpnI digestion. The treated product was then transformed into E. coli (pACYC-MseIM).

### 3. Selection of HpaI-HF

Selection of HpaI-HF was achieved using a method that was different to the previous examples. The cleavage activity and star activity were determined using lambda DNA substrate in NEB2 buffer. This HpaI has much more star activity in NEB2 than NEB4, and could be clearly observed in 5% glycerol.

HpaI(Y29F) and HpaI(E56A) were both preferred mutations with reduced star activity. HpaI(E56A) was labelled HpaI-HF.

### 4. Comparison of HpaI-HF and WT HpaI

The FIs of HpaI-HF and WT HpaI were determined separately on lambda DNA substrate with diluent A in each of NEB1-4 buffers. The comparison is shown in FIG. 28, and the result is listed in Table 27 (below).

TABLE 27
Comparison of HpaI-HF and WT HpaI

| | HpaI-HF | | WT HpaI | | |
|---|---|---|---|---|---|
| | | Fidelity | | Fidelity | Improvement |
| Buffer | Activity | Index | Activity | Index | Factor |
| NEB1 | 3.1% | ≥32 | 6.3% | 32 | ≥1 |
| NEB2 | 100% | ≥2000 | 25% | 1 | ≥2000 |
| NEB3 | 12.5% | 2 | 12.5% | 2 | 1 |
| NEB4 | 50% | ≥2000 | 100% | 16 | ≥120 |

HpaI-HF performed best in NEB2, in which the preferred FI was ≥2000; WT PciI performed best in NEB4, in which the preferred FI was 16. The overall FI improvement factor was ≥2000/16=120.

### Example 22: Engineering of High Fidelity AgeI

### 1. Expression of AgeI

AgeI was expressed in E. coli transformed with pRRS-AgeIRM and psyx20-lacIq. pRRS is a pUC19 derivative plasmid, psyx20-lacIq is a low copy compatible plasmid with lacI expressed under lacIq promoter. The cells were grown at 37° C. in LB with Amp and Kan to 200 Klett units, and then induced at 25° C. with 0.5 mM IPTG overnight. The expression of AgeI was extremely difficult to achieve because it was unstable.

### 2. Mutagenesis of AgeI

149 amino acid residues in AgeI were mutated with Cys, Asp, Glu, Phe, His, Lys, Met, Asn, Gln, Arg, Ser, and Thr. Trp was changed to Ala and Tyr to Phe. These were: 2, 4, 6, 7, 9, 14, 16, 18, 19, 21, 22, 23, 24, 25, 26, 27, 29, 30, 31, 32, 37, 38, 40, 42, 43, 44, 45, 49, 51, 53, 55, 56, 58, 60, 62, 64, 65, 67, 68, 69, 72, 73, 75, 77, 78, 79, 82, 83, 85, 86, 87, 88, 90, 91, 92, 94, 96, 97, 102, 103, 104, 105, 110, 111, 114, 116, 119, 120, 122, 123, 128, 129, 130, 134, 135, 138, 139, 140, 142, 144, 146, 147, 148, 152, 153, 155, 157, 159, 166, 168, 170, 173, 174, 176, 177, 178, 182, 183, 185, 186, 188, 192, 195, 198, 200, 201, 206, 211, 212, 214, 217, 219, 220, 222, 223, 224, 225, 226, 227, 229, 231, 233, 234, 235, 237, 238, 239, 240, 241, 243, 245, 247, 248, 250, 251, 253, 255, 256, 258, 260, 262, 265, 266, 267, 268, 269, 271, 272

The methods were the same as in the previous examples using inverse PCR followed by DpnI digestion. The treated product was then transformed into E. coli (psyx20-lacIq).

The numbers above correspond to amino acid positions in the AgeI protein sequence (SEQ ID NO:79).

### 3. Selection of AgeI-HF

Selection of AgeI-HF was achieved using a method that was similar to the previous examples. The standard activity check used pXba with 5% glycerol in NEB4 and the star activity check was on pXba in NEB4 buffer with 39% glycerol. Because of the difficulty of the expression system, this selection was repeated eight times before meaningful mutants were obtained. Two mutants, S201A and R139A had reduced star activity and R139A was labelled AgeI-HF.

### 4. Comparison of AgeI-HF and WT AgeI

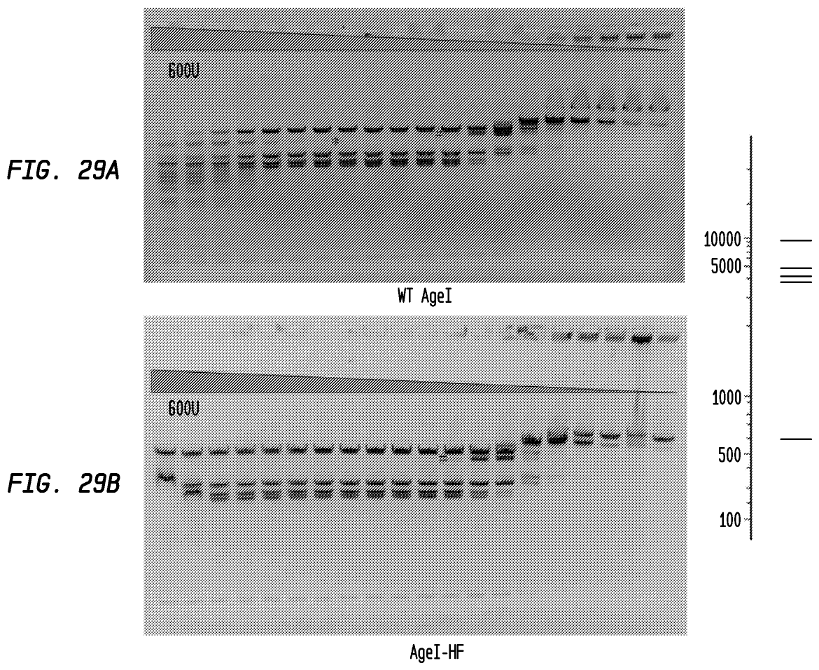

The FIs of AgeI-HF and WT AgeI were determined separately on pXba substrate with diluent A in each of NEB1-4 buffers. The comparison is shown in FIG. 29, and the result is listed in Table 28 (below).

TABLE 28
Comparison of AgeI-HF and WT AgeI

| | AgeI-HF | | WT AgeI | | |
|---|---|---|---|---|---|
| | | Fidelity | | Fidelity | Improvement |
| Buffer | Activity | Index | Activity | Index | Factor |
| NEB1 | 100% | ≥500 | 100% | 16 | ≥32 |
| NEB2 | 50% | ≥250 | 50% | 8 | ≥32 |
| NEB3 | 6.3% | ≥16 | 12.5% | 64 | ≥0.25 |
| NEB4 | 100% | ≥250 | 50% | 8 | ≥32 |

AgeI-HF performed best in NEB1, and NEB4, in which the preferred FI was ≥500; WT AgeI performed best in NEB3, in which the preferred FI was 16. The overall FI improvement factor was ≥500/16=32.

### Example 23: Engineering of High Fidelity BsmBI

### 1. Expression of BsmBI

BsmBI was expressed in E. coli transformed with pACYC-BsmAIM, ptaczz2-BsmBIR and psyx20-lacIq. Ptaczz2 is a pUC19 derivative plasmid which carries a inducible ptac promoter, pACYC is a low copy compatible plasmid. BsmAIM (GTCTC) covers BsmBI (CGTCTC) specificity. The psyx20-lacIq is a low copy vector with strong express of lacI. The cells were grown at 37° C. then induced in LB with Amp, Cam and Kan.

### 2. Mutagenesis of BsmBI

358 amino acid residues in BsmBI were mutated with Cys, Asp, Glu, Phe, His, Lys, Met, Asn, Gln, Arg, Ser, and Thr. Trp was changed to Ala and Tyr to Phe. These were: 8, 9, 12, 13, 14, 15, 17, 18, 19, 20, 21, 24, 25, 26, 27, 28, 29, 31, 33, 37, 38, 40, 42, 43, 44, 46, 47, 49, 50, 51, 52, 53, 54, 55, 56, 57, 58, 60, 61, 62, 63, 64, 65, 66, 67, 69, 70, 72, 76, 78, 79, 80, 81, 82, 83, 84, 88, 91, 93, 95, 96, 98, 99, 101, 103, 104, 105, 106, 109, 110, 111, 113, 114, 115, 117, 118, 119, 120, 121, 122, 124, 126, 127, 128, 130, 131, 132, 133, 134, 135, 138, 141, 143, 144, 145, 147, 149, 150, 154, 155, 157, 158, 160, 162, 163, 164, 165, 166, 167, 168, 169, 172, 174, 175, 176, 177, 179, 180, 181, 182, 184, 185, 186, 188, 189, 191, 194, 195, 197, 200, 201, 203, 205, 206, 207, 208, 211, 212, 213, 214, 215, 216, 217, 220, 221, 222, 223, 224, 225, 226, 228, 229, 230, 231, 232, 233, 235, 236, 237, 238, 239, 240, 242, 243, 247, 250, 251, 252, 257, 258, 260, 262, 263, 264, 265, 266, 268, 269, 271, 273, 274, 279, 280, 282, 283, 284, 287, 288, 289, 292, 294, 295, 296, 297, 299, 300, 301, 302, 303, 304, 305, 306, 307, 309, 310, 313, 314, 315, 316, 317, 318, 320, 321, 324, 325, 326, 328, 331, 332, 333, 334, 335, 336, 339, 340, 341, 342, 343, 344, 345, 348, 349, 351, 352, 353, 355, 356, 357, 358, 360, 361, 363, 364, 366, 367, 368, 370, 372, 375, 376, 377, 379, 381, 382, 384, 385, 386, 388, 389, 390, 393, 394, 395, 396, 398, 399, 400, 401, 402, 403, 404, 405, 406, 407, 408, 409, 410, 411, 412, 413, 414, 418, 421, 424, 425, 426, 428, 430, 431, 432, 433, 434, 436, 437, 438, 439, 442, 443, 444, 445, 446, 446, 448, 449, 450, 451, 452, 453, 454, 457, 458, 460, 461, 462, 464, 466, 467, 468, 470, 471, 472, 473, 474, 477, 478, 479, 480, 482, 483, 484, 485, 486, 487, 488, 489, 491, 492, 495, 496, 497, 498, 499, 500, 502, 503, 504, 505, 506, 507, 510, 511, 515, 516, 517, 518, 519, 522, 523.

The numbers above correspond to amino acid positions in the BsmBI protein sequence (SEQ ID NO:81).

The methods were the same as in the previous examples using inverse PCR followed by DpnI digestion. The treated product was then transformed into E. coli (pACYC-BsmAIM, psyx20-lacIq).

### 3. Selection of BsmBI-HF

Selection of BsmBI-HF was achieved using a method that was similar to the previous examples. The cleavage was determined using lambda DNA with 5% glycerol in NEB4 and the star activity was determined using Litmus28i in NEB4 buffer with 39% glycerol. Preferred mutants included H230A, D231A and N185Y/R232A. N185Y/R232A was labeled BsmBI-HF.

### 4. Comparison of BsmBI-HF and WT BsmBI

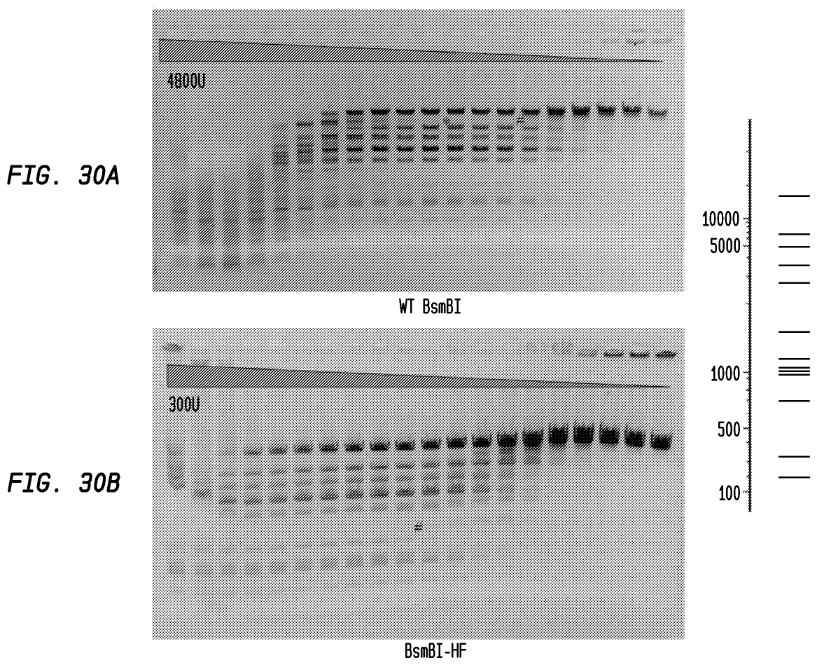

The FIs of BsmBI-HF and WT BsmBI were determined separately on lambda DNA substrate with diluent A in each of NEB1-4 buffers. The comparison is shown in FIG. 30, and the result is listed in Table 29 (below).

TABLE 29
Comparison of BsmBI-HF and WT BsmBI

| | BsmBI-HF | | WT BsmBI | | |
|---|---|---|---|---|---|
| | | Fidelity | | Fidelity | Improvement |
| Buffer | Activity | Index | Activity | Index | Factor |
| NEB1 | 100% | 32 | 12.5% | 1 | 32 |
| NEB2 | 100% | ≥500 | 50% | 8 | ≥64 |
| NEB3 | 12.5% | ≥64 | 100% | 120 | ≥0.5 |
| NEB4 | 100% | ≥500 | 25% | 4 | ≥120 |

BsmBI-HF performed best in NEB1, NEB2 and NEB4, in which the preferred FI was ≥500; WT BsmBI performed best in NEB3, in which the preferred FI was 120. The overall FI improvement factor was ≥500/120=4.

### Example 24: Engineering BsPQI Variants with Reduced Star Activity

### 1. Site-Directed Mutagenesis of BspQI Restriction Endonuclease

E. coli was transformed with pSX33-EarIM1M2 and pZZlacI-PspQI that was Km^{R} and Amp^{R}). M.EarI (CTCTTC) also modifies BspQI site (GCTCTTC) and therefore pSX33-earIM1M2 (FIGS. 17 and 18) was used to co-transform and modify the E. coli chromosome. The WT amino acid sequence is shown in FIG. 16.

122 charged or non-charged amino acid residues in BspQI (Arg, Lys, His, Glu, Asp, Gln, Asn, Cys) were changed to Ala by site-directed mutagenesis. PCR was carried out under the following conditions: DNA denaturation, 98° C. for 30 sec, 1 cycle; DNA denaturation/primer annealing/extension, 98° C. for 10 sec, 55° C. to 65° C. for 30 sec, 72° C. for 2 min, for PCR 18 cycles; 72° C. for 15 min, 1 cycle. In 100 μl reactions, 2 units of Phusion™ DNA polymerase (NEB, Ipswich, MA), 1 mM dNTP, 10 ng to 100 ng template DNA, 20 μl 5× reaction buffer, 0.04 μM primers, sterile water to 100 μl total volume.

PCR DNA was digested with DpnI to destroy template DNA (Dam methylated) and co-transformed with pSX33-earIM1M2 into E. coli. Individual transformants were cultured overnight (5 ml LB, 50 μg/ml Km^{R} and 100 μg/ml Amp^{R}) and split into two parts. One part (1.5 ml) was harvested by centrifugation and lysed by sonication in a sonication buffer (20 mM Tris-HCl, pH 7.5, 0.1 mM DTT, 50 mM NaCl, 10% glycerol). The cell extract was heated at 50° C. for 1 h and denatured E. coli proteins were removed by centrifugation. The clarified lysate was assayed for restriction activity and star activity on pUC19 DNA.

BspQI star activity assay condition: 1 μg pUC19 DNA, 5 μl of 10×NEB buffer 1, 25% DMSO, 2.5 μl clarified cell extract, sterile deionized water to 50 μl total volume and incubated at 50° C. for 1 h. Digested DNA was resolved by electrophoresis in 0.8 to 1% agarose gel.

The second part of the cell culture (uninduced) was harvested and plasmid DNA was prepared by Qiagen spin column purification procedure (Qiagen, Valencia, CA). The bspQIR alleles were sequenced by Big-dye dideoxy-terminator sequencing method to confirm the desired mutations. After identification of reduced star activity mutants, fresh transformants were obtained and IPTG-induced cultures were made. Restriction and star activity was assayed again to confirm the reduced star activity in comparison with the WT enzyme in all four buffers.

Among the 122 BspQI mutants constructed by site-directed mutagenesis, two BspQI variants, R388A and K279A, display reduced star activity. The star activity of R388A was reduced approximately 16-fold in buffer 1 and 10% glycerol. However, R388A still displayed star activity at high enzyme concentration. BspQI variant K279A also displayed reduced star activity (>8-fold improvement in reduced star activity).

To further reduce star activity at high enzyme concentration, R388 and K279 were substituted for Phe, Pro, Tyr, Glu, Asp, or Leu. IPTG-induced cell extracts of various R388X, and K279× mutants were assayed for restriction and star activity. It was found that R388F or K279P displayed the minimal star activity in either cell extracts or purified enzyme. The specific activity was not affected by the amino acid substitutions.

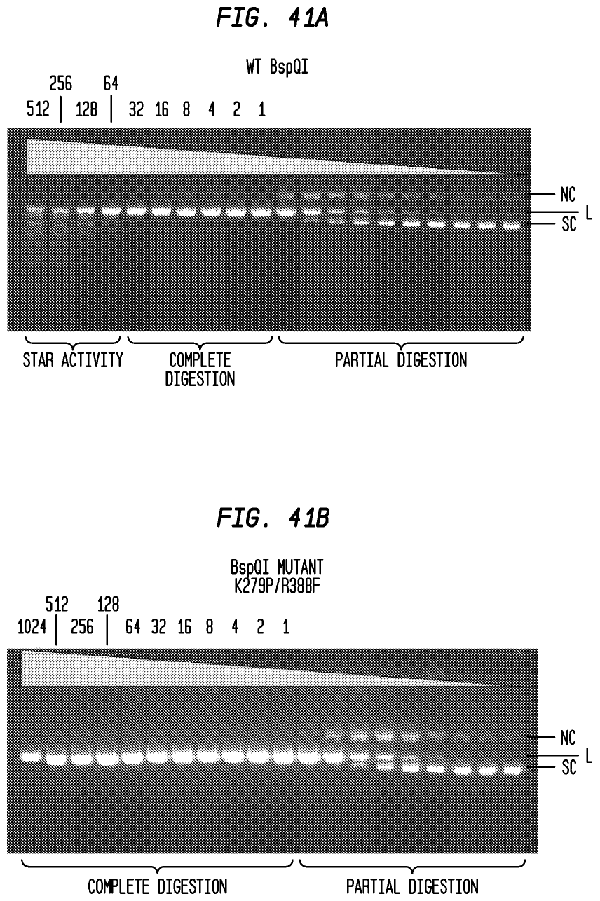

To still further reduce BspQI star activity, the two amino acid substitutions were combined into one mutant enzyme (double mutant, K279P/R388F) by site-directed mutagenesis. This double mutant lacks star activity in buffer 1 and buffer 2 with 10% glycerol (FIG. 41B).

TABLE 30
BspQI-HF vs WT BspQI

| | BspQI-HF | | WT BspQI | | |
|---|---|---|---|---|---|
| | | Fidelity | | Fidelity | Improvement |
| Buffer | Activity | Index | Activity | Index | Factor |
| NEB1 | 25% | ≥1000 | 12.5% | 2 | ≥1000 |
| NEB2 | 25% | ≥1000 | 100% | 16 | ≥64 |
| NEB3 | 1.3% | ≥64 | 100% | 32 | ≥2 |
| NEB4 | 100% | ≥4000 | 50% | 4 | ≥1000 |

### Example 25: Engineering SapI Variants with Reduced Star Activity

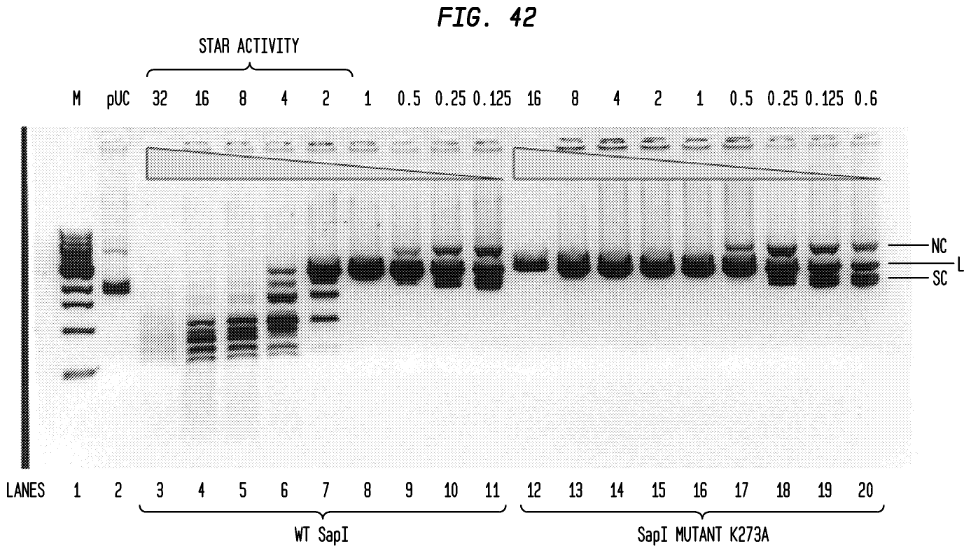

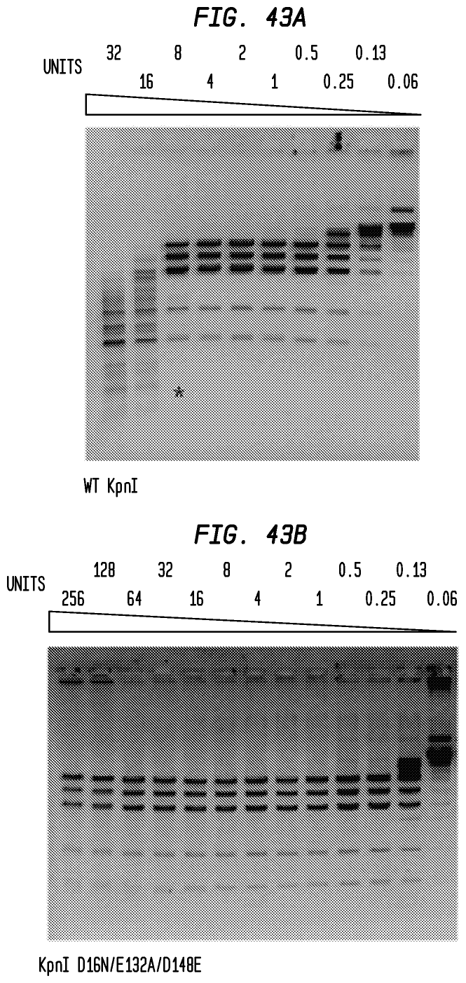

The conserved K279 and R388 amino acid residues were found in BspQI where corresponding positions are K273 and R380 in SapI. A 6×His tagged SapI expression clone was first constructed in pUC19. The SapI expression strain was E. coli transformed with pSX33-earIM1M2 and pUC-SapI that was Km^{R} and Amp^{R}. Lys273 to Pro (K273P) and Arg 380 to Phe (R380F) amino acid substitutions were introduced into SapI by site-directed mutagenesis. SapI single mutant R380A was also constructed. Both SapI variants R380A and K273P/R380F showed reduced star activity when restriction activity and star activity reactions were performed (FIG. 42).

PCR, transformation, plasmid DNA preparation, and enzyme activity assay were carried out as described for BspQI, except that SapI activity was determined at 37° C. The 6× His-tagged SapI variant K273P/R380F was purified through Ni-NTA column chromatography and shown to display diminished star activity in the presence of 25% DMSO or 5% glycerol.

### Example 26: Engineering KpnI High Fidelity Mutants

KpnI, which contains two activities, has been changed into a mutant with lower star activity (International Publication No. WO 07/027464). The example below describes novel mutants with improved star activity and similar cleavage activity to the wild-type.

Charged amino acid residues except the catalytic residues, (Asp, Glu, Arg, Lys and His) or polar amino acids (Ser, Thr, Tyr, Asn, Gln, Phe, Trp, Cys and Met) were individually mutated to Alanine.

Mutagenesis was carried out by inverse PCR using primers bearing the desired mutations. In general, inverse PCR was performed using 0.4 mM of each of the 4 dNTP, 1× ThermoPol Buffer (NEB), 20 ng of template DNA, 40 μmol of each of the primers and 4 U of Vent DNA polymerase (NEB) in a final volume of 100 μL.

Plasmids pUC19 or pAGR3 containing the KpnIR under the control of Plac or Ptac promoter, respectively, were used as template. PCRs were done with a temperature scheme of 94° C. for 4 minutes followed by 25 cycles of 94° C. for 30 seconds for denaturation, 55° C. for 30 seconds for annealing and 72° C. for 5 minutes for extension. The cycles were followed by incubation at 72° C. for 7 minutes before the reactions were treated by 20 U of DpnI (NEB) at 37° C. for 1 hour to degrade the template DNA. After inactivating DpnI at 80° C. for 20 minutes, 2 μl of the reaction was used to transform 50 μL of chemically competent NEB5alpha (NEB) pre-transformed by pSYX20-KpnIM. The transformed bacteria were plated out onto LB plates containing 100 μg/ml of ampicillin and 30 μg/ml of kanamycin, and incubated at 37° C. for 12 to 15 hours. Three to four colonies of each construct were cultured in 1 ml of LB containing 100 μg/ml of ampicillin and 30 μg/ml of kanamycin at 37° C. for 12 to 15 hours with shaking of 200 rpm. The cultures were spun down and resuspended in 0.2 ml of sonication buffer (20 mM Tris-HCl, pH 8.3, 50 mM NaCl, 1 mM EDTA, 1 mM PMSF.) The resuspended cells were sonicated for 20 seconds, followed by centrifugation at 13,000 rpm at 4° C. for 5 min. Dilutions of the supernatant were made and 5 μL of which were assayed for KpnI cleavage activity.

For the screening of the mutants, an activity assay reaction was performed using 5 μL of 10- or 100-fold dilutions of the lysate supernatant, 0.5 ug of pXba DNA (NEB) and 1× NEBuffer 4 in a total volume of 50 μL. After incubating at 37° C. for 1 hour, the assay reactions were stopped by adding 10 μl of 6×DNA loading dye and analyzed by electrophoresis through 0.8% agarose gels in 1×TBE. Mutants that showed increased overall cleavage activity compared with the parent enzyme (KpnI D148E) were assayed for reduced star activity in a buffer containing 25% DMSO.

The assay was performed using 5 μL of dilutions of the enzymes incubated with 1 μg of pXba DNA (NEB) in the presence of 5% glycerol and 0.2 mg/ml of BSA in 1×NEBuffer 4 (total volume=50 μL). After incubation at 37° C. for 1 hour, the reactions were treated by 20 μg of proteinase K (NEB) at 37° C. for 15 minutes and then analyzed by electrophoresis through 0.8% agarose gels in 1×TBE. Divalent metal-dependent assays were carried out at 37° C. for 1 hour, using 50 U of enzyme and 1 μg of pXba DNA in a buffer containing 20 mM Tris HCl, 50 mM NaCl, pH 7.9, 1 mM DTT and increasing concentration of MgSO₄/CaCl₂)/MnCl2, followed by electrophoresis through 0.8% agarose gels in 1×TBE. The total reaction volume is 50 μL.

The result of random mutagenesis, was a preferred mutant, KpnI D148E, which showed lower star activity than the wild-type enzyme and KpnI D163I/K165A mutant described previously (International Publication No. WO 07/027464). However, KpnI D148E displayed star activity at high enzyme concentration. Double and triple mutants were constructed in D148E background to reduce this observed star activity. KpnI (D16N/E132A/D148E) was found to have lower star activity and higher specificity activity than mutant D148E. Amino acid substitutions D148 to E and E132 to A were introduced by site-directed mutagenesis. The D16 to N mutation was introduced by PCR. In standard reaction condition, one-hour incubation of purified enzymes with substrate DNA pXba (containing 6 KpnI sites) at 37° C.), resulted in the reduced star activity shown in Table 31.

TABLE 31
Fidelity indices for KpnI mutants

| | KpnI-HF | | WT KpnI | | |
|---|---|---|---|---|---|
| | | Fidelity | | Fidelity | Improvement |
| Buffer | Activity | Index | Activity | Index | Factor |
| NEB1 | 100% | ≥4000 | 100% | 16 | ≥250 |
| NEB2 | 50% | ≥2000 | 25% | 16 | ≥64 |
| NEB3 | 6.3% | ≥250 | 6.3% | 8 | ≥32 |
| NEB4 | 100% | ≥4000 | 50% | 4 | ≥1000 |

No star activity of pXba was observed for mutant D16N/E132A/D148E up to 4000U. Star activity observed for pBR322 substrate, which bears no KpnI site, was also diminished when cleaved with KpnI D148E and D16N/E132A/D148E.

### Example 27: Engineering of High Fidelity BsaI

### 1. Expression of BsaI

BsaI was expressed in E. coli transformed with pACYC-BsmAIM, pUC19-BsaIR and psyx20-lacIq. pACYC is a low copy compatible plasmid. BsmAIM(GTCTC) covers BsmBI specificity (CGTCTC). The psyx20-lacIq is a low copy vector with strong express of lacI. The cells were grown at 37° C. then induced in LB with Amp, Cam and Kan.

### 2. Mutagenesis of BsaI

The amino acid of BsaI is similar to that of BsmBI. 11 amino acids around and at the corresponding previous effective site is mutated as R229A, S230A, Y23IF, T232A, T233A, D234A, R235A, R236A, F238A, E239A, Y240F.

The methods were the same as in the previous examples using inverse PCR followed by DpnI digestion. The treated product was then transformed into E. coli (pACYC-BsmAIM, psyx20-lacIq).

### 3. Selection of BsaI-HF

Selection of BsaI-HF was achieved using a method that was similar to the previous examples. The standard activity check used lambda DNA with 5% glycerol in NEB4 and the star activity check was litmus28i in NEB4 buffer with 39% glycerol. One mutant, Y231F, out of the 11 designed ones, reduced star activity and labeled as BsaI-HF.

### 4. Comparison of BsaI-HF and WT BsaI

The FIs of BsaI-HF and WT BsaI were determined separately on lambda DNA with diluent A in NEB1-4 buffers. The result is listed in Table 32 (below).

TABLE 32
Comparison of BsaI-HF and WT BsaI

| | BsaI-HF | | WT BsaI | | |
|---|---|---|---|---|---|
| | | Fidelity | | Fidelity | Improvement |
| Buffer | Activity | Index | Activity | Index | Factor |
| NEB1 | 50% | ≥4000 | 25% | 8 | ≥500 |
| NEB2 | 100% | ≥8000 | 100% | 120 | ≥64 |
| NEB3 | 100% | 120 | 25% | 16 | 8 |
| NEB4 | 100% | ≥8000 | 100% | 32 | ≥250 |

BsaI-HF performed best in NEB2, NEB3 and NEB4, in which the best FI was ≥8000; WT BsaI performed best in NEB2 and NEB4, in which the best FI was 120. So the overall FI improvement factor was ≥8000/120=≥64.

What is claimed is:

1-43. (canceled)

44. A composition comprising a variant SbfI restriction endonuclease, wherein the variant SbfI restriction endonuclease comprises an amino acid sequence having the following substitution:
a substitution at a position corresponding to 251 in SEQ ID NO: 96.

45. A composition according to claim 53, wherein the substitution corresponding to position 251 is K251A.
